(19) United States
(12) Patent Application Publication
(10) Pub. No.: US 2015/0150962 A1
(43) Pub. Date: Jun. 4, 2015

(54) Chicken Astrovirus Responsible for Runting Stunting Syndrome

(71) Applicant: University of Georgia Research Foundation, Inc., Athens, GA (US)

(72) Inventors: HOLLY S. SELLERS, BISHOP, GA (US); EGBERT MUNDT, WATKINSVILLE, GA (US)

(21) Appl. No.: 14/612,495

(22) Filed: Feb. 3, 2015

Related U.S. Application Data
(63) Continuation-in-part of application No. PCT/US2013/053454, filed on Aug. 2, 2013.
(60) Provisional application No. 61/679,325, filed on Aug. 3, 2012.

(51) Int. Cl. A61K 39/12; G01N 33/68; C12N 7/00

(52) U.S. Cl. CPC A61K 39/12; C12N 7/00; G01N 33/6854; A61K 2039/552

(57) ABSTRACT

The present invention includes isolated astroviruses capable of reproducing the full Runting Stunting Syndrome (RSS) disease spectrum in poultry, including a chicken astrovirus isolated from the gut of chickens (CkAstV-Gut), a cell culture chicken astrovirus CkAstV-p5, and a chicken astrovirus isolated after back passage in chicken (CkAstV-p5-Ckp5), and infected cell lines, cell culture supernatants, polynucleotide sequences, vectors, polypeptides, compositions, and vaccines thereof. The present invention also includes diagnostic methods based on such isolated viruses and infected cell lines, cell culture supernatants, polynucleotide sequences, vectors, and polypeptides thereof, and methods of protecting poultry, including chickens, against RSS by the administration of such isolated viruses and infected cell lines, cell culture supernatants, polynucleotide sequences, vectors, polypeptides, compositions, and vaccines thereof.

### CONTINUING APPLICATION DATA

This application is a continuation-in-part application of International Application No. PCT/US2013/053454, filed Aug. 2, 2013, which claims the benefit of U.S. Provisional Application Ser. No. 61/679,325, filed Aug. 3, 2012, each of which is incorporated by reference herein.

### SEQUENCE LISTING

This application contains a Sequence Listing electronically submitted to the United States Patent and Trademark Office via EFS-Web as an ASCII text file entitled “235-02140120_SequenceListing_ST25.txt” having a size of 99 kilobytes and created on Jan. 16, 2015. The information contained in the Sequence Listing is incorporated by reference herein.

### BACKGROUND

The runting stunting syndrome (RSS) in chickens is an economically devastating disease. The disease, also known as MAS, infectious stunting syndrome, broiler runting syndrome, pale bird syndrome, or helicopter syndrome, is a transmissible disease characterized by a stunted growth of chickens, an increased feed conversion rate, and poor flock uniformity in the size of the chickens. With RSS, chickens develop diarrhea and show a higher susceptibility to other diseases. Furthermore, cystic enteropathic lesions have been described as one of the hallmarks of the disease. RSS is an economically devastating disease for the poultry industry. Despite descriptions of runting stunting syndrome (RSS) in broiler chickens dating back over 40 years, the etiology is unknown. Available diagnostic tests are very limited and there is no vaccine to prevent or mitigate the disease. Thus, there is a need for improved diagnostic and therapeutic reagents and methods for the detection, treatment, and prevention of RSS.

### SUMMARY OF THE INVENTION

The present invention includes an isolated chicken astrovirus, the chicken astrovirus having a full length genomic sequence having at least about 75% sequence identity, at least about 80% sequence identity, at least about 85% sequence identity, at least about 90% sequence identity, at least about 95% sequence identity, at least about 98% sequence identity, or at least about 99% sequence identity to the genomic sequence of the cell culture chicken astrovirus CkAstV-p5 (SEQ ID NO:2), the genomic sequence the chicken astrovirus after back passage in chicken CkAstV-p5-Ckp5 (SEQ ID NO:3), or the genomic sequence of the chicken astrovirus isolated from the gut of chickens CkAstV-Gut (SEQ ID NO:1), and attenuations and derivatives thereof.

The present invention includes an isolated chicken astrovirus, the chicken astrovirus having a full length genomic sequence of the genomic sequence of the cell culture chicken astrovirus CkAstV-p5 (SEQ ID NO:2), the genomic sequence of the chicken astrovirus after back passage in chicken CkAstV-p5-Ckp5 (SEQ ID NO:3), or the genomic sequence of the chicken astrovirus isolated from the gut of chickens CkAstV-Gut (SEQ ID NO:1), and attenuations and derivatives thereof. The present invention includes an isolated chicken astrovirus, the chicken astrovirus including an open reading frame 1a (ORF1a) with an amino acid sequence having at least about 75% sequence identity, at least about 80% sequence identity, at least about 85% sequence identity, at least about 90% sequence identity, at least about 95% sequence identity, at least about 96% sequence identity, at least about 97% sequence identity, at least about 98% sequence identity, or at least about 99% sequence identity to SEQ ID NO:4, SEQ ID NO:5, or SEQ ID NO:6, and attenuations and derivatives thereof.

The present invention includes an isolated chicken astrovirus, the chicken astrovirus including an open reading frame 1a (ORF1a) with amino acid sequence SEQ ID NO:4, SEQ ID NO:5, or SEQ ID NO:6, and attenuations and derivatives thereof.

The present invention includes an isolated chicken astrovirus, the chicken astrovirus including an open reading frame 1b (ORF1b) with an amino acid sequence with at least about at least about 75% sequence identity, at least about 80% sequence identity, at least about 85% sequence identity, at least about 90% sequence identity, at least about 95% sequence identity, at least about 96% sequence identity, at least about 97% sequence identity, at least about 98% sequence identity, or at least about 99% sequence identity to SEQ ID NO:7, SEQ ID NO:8, or SEQ ID NO:9, and attenuations and derivatives thereof.

The present invention includes an isolated chicken astrovirus, the chicken astrovirus including an open reading frame 1b (ORF1b) with amino acid sequence SEQ ID NO:7, SEQ ID NO:8, or SEQ ID NO:9, and attenuations and derivatives thereof.

The present invention includes an isolated chicken astrovirus, the chicken astrovirus including an open reading frame 2 (ORF2) with an amino acid sequence with at least about 75% sequence identity, at least about 80% sequence identity, at least about 85% sequence identity, at least about 90% sequence identity, at least about 95% sequence identity, at least about 96% sequence identity, at least about 97% sequence identity, at least about 98% sequence identity, or at least about 99% sequence identity SEQ ID NO:10, SEQ ID NO:11, or SEQ ID NO:12, and attenuations and derivatives thereof.

The present invention includes an isolated chicken astrovirus, the chicken astrovirus including an open reading frame 2 (ORF2) with amino acid sequence SEQ ID NO:10, SEQ ID NO:11, or SEQ ID NO:12, and attenuations and derivatives thereof.

In some aspects of a virus of the present invention, the virus is attenuated, inactivated, and/or killed.

The present invention includes a cell culture supernatant, the cell culture supernatant including a chicken astrovirus including a full length genomic sequence with at least about 75% sequence identity, at least about 80% sequence identity, at least about 85% sequence identity, at least about 90% sequence identity, at least about 95% sequence identity, at least about 98% sequence identity, or at least about 99% sequence identity to the genomic sequence of the cell culture chicken astrovirus CkAstV-p5 (SEQ ID NO:2), to the genomic sequence of the chicken astrovirus after back passage in chicken CkAstV-p5-Ckp5 (SEQ ID NO:3), or the genomic sequence of the chicken astrovirus isolated from the gut of chickens CkAstV-Gut (SEQ ID NO:1), and attenuations and derivatives thereof.

The present invention includes a cell culture supernatant, the cell culture supernatant including a chicken astrovirus including a full length genomic sequence of the cell culture chicken astrovirus CkAstV-p5 (SEQ ID NO:2), the genomic sequence the chicken astrovirus after back passage in chicken CkAstV-p5-Ckp5 (SEQ ID NO:3), or the genomic sequence of the chicken astrovirus isolated from the gut of chickens CkAstV-Gut (SEQ ID NO:1), and attenuations and derivatives thereof.

The present invention includes a cell culture supernatant, the cell culture supernatant including a chicken astrovirus with an open reading frame 1a (ORF1a) with an amino acid sequence with at least about 75% sequence identity, at least about 80% sequence identity, at least about 85% sequence identity, at least about 90% sequence identity, at least about 95% sequence identity, at least about 96% sequence identity, at least about 97% sequence identity, at least about 98% sequence identity, or at least about 99% sequence identity at least about 90% sequence identity to SEQ ID NO:4, SEQ ID NO:5, or SEQ ID NO:6, and attenuations and derivatives thereof.

The present invention includes a cell culture supernatant, the cell culture supernatant including a chicken astrovirus with amino acid sequence SEQ ID NO:4, SEQ ID NO:5, or SEQ ID NO:6, and attenuations and derivatives thereof.

The present invention includes a cell culture supernatant, the cell culture supernatant including a chicken astrovirus having an open reading frame 1b (ORF1b) with an amino acid sequence with at least about 75% sequence identity, at least about 80% sequence identity, at least about 85% sequence identity, at least about 90% sequence identity, at least about 95% sequence identity, at least about 96% sequence identity, at least about 97% sequence identity, at least about 98% sequence identity, or at least about 99% sequence identity to SEQ ID NO:7, SEQ ID NO:8, or SEQ ID NO:9, and attenuations and derivatives thereof.

The present invention includes a cell culture supernatant, the cell culture supernatant including a chicken astrovirus having an open reading frame 1b (ORF1b) with amino acid sequence SEQ ID NO:7, SEQ ID NO:8, or SEQ ID NO:9, and attenuations and derivatives thereof.

The present invention includes a cell culture supernatant, the cell culture supernatant including a chicken astrovirus having an open reading frame 2 (ORF2) with an amino acid sequence with at least about 75% sequence identity, at least about 80% sequence identity, at least about 85% sequence identity, at least about 90% sequence identity, at least about 95% sequence identity, at least about 96% sequence identity, at least about 97% sequence identity, at least about 98% sequence identity, or at least about 99% sequence identity SEQ ID NO:10, SEQ ID NO:11, or SEQ ID NO:12, and attenuations and derivatives thereof.

The present invention includes a cell culture supernatant, the cell culture supernatant including a chicken astrovirus having an open reading frame 2 (ORF2) with amino acid sequence SEQ ID NO:10, SEQ ID NO:11, or SEQ ID NO:12, and attenuations and derivatives thereof.

The present invention includes an isolated chicken cell line, the cells infected with a chicken astrovirus having a full length genomic sequence with at least about 75% sequence identity, at least about 80% sequence identity, at least about 85% sequence identity, at least about 90% sequence identity, at least about 95% sequence identity, at least about 98% sequence identity, or at least about 99% sequence identity to the genomic sequence of the cell culture chicken astrovirus CkAstV-p5 (SEQ ID NO:2), the genomic sequence of the chicken astrovirus after back passage in chicken CkAstV-p5-Ckp5 (SEQ ID NO:3), or the genomic sequence of the chicken astrovirus isolated from the gut of chickens CkAstV-Gut (SEQ ID NO:1), or an attenuation or derivative thereof.

The present invention includes an isolated chicken cell line, the cells infected with a chicken astrovirus having a full length genomic sequence of the cell culture chicken astrovirus CkAstV-p5 (SEQ ID NO:2), of the chicken astrovirus after back passage in chicken CkAstV-p5-Ckp5 (SEQ ID NO:3), or of the chicken astrovirus isolated from the gut of chickens CkAstV-Gut (SEQ ID NO:1), or an attenuation or derivative thereof.

The present invention includes an isolated chicken cell line, the cells infected with a chicken astrovirus including an open reading frame 1a (ORF1a) with an amino acid sequence with at least about 75% sequence identity, at least about 80% sequence identity, at least about 85% sequence identity, at least about 90% sequence identity, at least about 95% sequence identity, at least about 96% sequence identity, at least about 97% sequence identity, at least about 98% sequence identity, or at least about 99% sequence identity to SEQ ID NO:4, SEQ ID NO:5, or SEQ ID NO:6, or an attenuation or derivative thereof.

The present invention includes an isolated chicken cell line, the cells infected with a chicken astrovirus including an open reading frame 1a (ORF1a) with amino acid sequence with SEQ ID NO:4, SEQ ID NO:5, or SEQ ID NO:6, or an attenuation or derivative thereof.

The present invention includes an isolated chicken cell line, the cells infected with a chicken astrovirus including an open reading frame 1b (ORF1b) with an amino acid sequence with at least about 75% sequence identity, at least about 80% sequence identity, at least about 85% sequence identity, at least about 90% sequence identity, at least about 95% sequence identity, at least about 96% sequence identity, at least about 97% sequence identity, at least about 98% sequence identity, or at least about 99% sequence identity SEQ ID NO:7, SEQ ID NO:8, or SEQ ID NO:9, or an attenuation or derivative thereof.

The present invention includes an isolated chicken cell line, the cells infected with a chicken astrovirus including an open reading frame 1b (ORF1b) with amino acid sequence with SEQ ID NO:7, SEQ ID NO:8, or SEQ ID NO:9, or an attenuation or derivative thereof.

The present invention includes an isolated chicken cell line, the cells infected with a chicken astrovirus including an open reading frame 2 (ORF2) with an amino acid sequence with at least about 75% sequence identity, at least about 80% sequence identity, at least about 85% sequence identity, at least about 90% sequence identity, at least about 95% sequence identity, at least about 96% sequence identity, at least about 97% sequence identity, at least about 98% sequence identity, or at least about 99% sequence identity SEQ ID NO:10, SEQ ID NO:11 or SEQ ID NO:12, or an attenuation or derivative thereof.

The present invention includes an isolated chicken cell line, the cells infected with a chicken astrovirus including an open reading frame 2 (ORF2) with amino acid sequence SEQ ID NO:10, SEQ ID NO:11 or SEQ ID NO:12, or an attenuation or derivative thereof.

In some aspects of a chicken cell line of the present invention, the cell line is LMH.

In some aspects of a chicken cell line of the present invention, the cells are a cell pellet.

The present invention includes an isolated polynucleotide sequence having at least about 75% sequence identity, at least about 80% sequence identity, at least about 85% sequence identity, at least about 90% sequence identity, at least about 95% sequence identity, at least about 98% sequence identity, or at least about 99% sequence identity to a chicken astrovirus (CkAst) genomic sequence with SEQ ID NO:1, SEQ ID NO:2, or SEQ ID NO:3, a truncation, or fragment thereof.

The present invention includes an isolated polynucleotide sequence having SEQ ID NO:1, SEQ ID NO:2, or SEQ ID NO:3, a truncation, or fragment thereof.

The present invention includes an isolated polynucleotide sequence having at least about 75% sequence identity, at least about 80% sequence identity, at least about 85% sequence identity, at least about 90% sequence identity, at least about 95% sequence identity, at least about 98% sequence identity, or at least about 99% sequence identity to a nucleotide sequence encoding: an ORF1a selected from SEQ ID NO:4, SEQ ID NO:5, or SEQ ID NO:6; an ORF1b selected from SEQ ID NO:7, SEQ ID NO:8, or SEQ ID NO:9; and/or an ORF2 selected from SEQ ID NO:10, SEQ ID NO:11, or SEQ ID NO:12.

The present invention includes an isolated polynucleotide sequence having a nucleotide sequence encoding an ORF1a selected from SEQ ID NO:4, SEQ ID NO:5, or SEQ ID NO:6; an ORF1b selected from SEQ ID NO:7, SEQ ID NO:8, or SEQ ID NO:9; and/or an ORF2 selected from SEQ ID NO:10, SEQ ID NO:11, or SEQ ID NO:12.

The present invention includes an isolated polypeptide having at least about 75% sequence identity, at least about 80% sequence identity, at least about 85% sequence identity, at least about 90% sequence identity, at least about 95% sequence identity, at least about 98% sequence identity, or at least about 99% sequence identity to the amino acid sequence SEQ ID NO:4, SEQ ID NO:5, SEQ ID NO:6, SEQ ID NO:7, SEQ ID NO:8, SEQ ID NO:9, SEQ ID NO:10, SEQ ID NO:11, or SEQ ID NO:12.

The present invention includes an isolated polypeptide having an amino acid sequence selected from SEQ ID NO:4, SEQ ID NO:5, SEQ ID NO:6, SEQ ID NO:7, SEQ ID NO:8, SEQ ID NO:9, SEQ ID NO:10, SEQ ID NO:11, or SEQ ID NO:12.

The present invention includes a vector including a polynucleotide sequence of the present invention. In some aspect the vector is a vaccine vector. In some aspects the vector is a Newcastle Disease based vaccine vector.

The present invention includes an isolated polypeptide encoded by a polynucleotide of the present invention.

The present invention includes compositions including a virus, cell line, cell pellet, supernatant, polynucleotide sequence, vector, or polypeptide of the present invention, as described herein.

The present invention includes immunological compositions for raising antibodies in poultry, the composition including a virus, cell line, cell pellet, supernatant, polynucleotide sequence, vector, or polypeptide of the present invention.

The present invention includes a vaccine including a virus, cell line, cell pellet, supernatant, polynucleotide sequence, vector, or polypeptide of the present invention.

In some aspects, a composition or vaccine of the present invention further includes an adjuvant.

In some aspects, a composition or vaccine of the present invention further includes an antigenic determinant from one or more additional pathogens infectious to poultry.

The present invention includes diagnostic kits including one or more of a virus, cell line, cell pellet, supernatant, polynucleotide sequence, vector, and/or polypeptide of the present invention.

The present invention includes an antibody that binds to a virus or polypeptide of the present invention. In some aspects, the antibody is a monoclonal antibody.

The present invention includes a diagnostic kit including an antibody that binds to a virus or polypeptide of the present invention.

The present invention includes a method of detecting exposure to runting-stunting syndrome (RSS) in a bird, the method including that an antisera sample obtained from the bird specifically binds to a virus, cell line, cell pellet, supernatant, and/or polypeptide of the present invention.

The present invention includes a method of detecting a runting stunting syndrome (RSS) infectious agent in a sample, the method including producing a polymerase chain reaction (PCR) amplification product with the primer pair as described herein. In some aspects, the primer pair includes SEQ ID NO:13 and SEQ ID NO:14.

The present invention includes a method of producing an anti-RSS immune response in poultry, the method including administering an isolated virus, cell line, cell pellet, supernatant, polynucleotide sequence, vector, and/or polypeptide of the present invention. In some aspects, immunity includes humoral and/or cellular immunity. In some aspects, immunity includes mucosal immunity.

The present invention includes a method of preventing RSS in poultry, the method including administering a composition including an isolated virus, cell line, cell pellet, supernatant, polynucleotide sequence, vector, and/or polypeptide of the present invention.

In some aspects of the methods of the present invention, administration includes injection, spraying, oral administration, or respiratory administration.

In some aspects of the methods of the present invention, administration induces mucosal immunity.

In some aspects of the methods of the present invention, administration includes in ovo administration. In some aspects, in ovo administration includes administration at about 13 days, about 14 days, about 15 days, about 16 days, about 17 days, about 18 days, about 19 days, about 20 days, or any range thereof.

The present invention includes a method of detecting a runting stunting syndrome (RSS) infectious agent in a sample, the method including detecting the hybridization of a polynucleotide of the present invention.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

### BRIEF DESCRIPTION OF THE FIGURES

FIG. 1 shows characterization of a chicken astrovirus in cell culture. (A) LMH cells were infected with CkAstV-p5 and indirect immunofluorescence was performed 24 h after infection using r-anti-ckAstV serum and goat-anti rabbit FITC-conjugated antibodies. (B) Western blot analysis of protein samples of i) LMH cell either infected (inf) with CkAstV-p5 or not (cont), and ii) Sf 9 cells infected (inf) with a recombinant baculovirus encoding for the capsid protein of a chicken astrovirus (Sellers et al., 2010 Vaccine, 28:1253-1263) or not infected cells (cont). The membranes were incubated either with the rabbit serum r-anti-CkAstV (dilution 1:10000) or the HRP-conjugated anti 6×His monoclonal antibody (dilution 1:5000). A ladder (M) for protein molecular weights (Bionexus 20 kDa dual color prestained protein marker) was indicated on the left part of the gel. (C) LMH cells cultured in a 24-well tissue culture plate were infected with CkAstV-p5 at 100 TCID₅₀/well. Supernatants and cells were removed at the indicated time points and the TCID₅₀/100 μl was determined. The diamonds represent the average of three independent studies and the standard deviation is shown by bars. (D) Virus titers were determined individually at the indicated time points after infection (h p.i.) for the supernatant and LMH cells infected with 100 TCID₅₀/well of CkAstV-p5. The diamonds represent the average of three independent studies and the standard deviation was shown as bars at each time point.

FIG. 2 shows infection of broiler chickens with a chicken astrovirus resulted in viral replication in the crypt of the duodenum. Ten one-day-old broiler chickens were infected with either gut content of RSS affected chickens (RSS) or a chicken astrovirus isolated in cell culture (CkAstV). One group of chickens was not inoculated and served as a negative control. (A) The body weight of the chickens. *p<0.05. **p<0.01. (B) The number of cystic lesion in the duodenal loop of the chickens. (C) The presence of viral RNA as detected by in situ hybridization (ISH) was determined from five chickens each at day 5 and 12 after infection (d p.i.). The presence of viral RNA was documented in the crypt epithelial cells of a chicken infected with the chicken astrovirus by ISH. Bar=100 μm. (D) The ISH score was estimated based on the following scale: 0 no signals; 1=five signals per high-power field; 2=five to 15 signals per high-power field; 3>15 signals per high-power field.

FIG. 3 shows dynamics of viral RNA in the duodenum of chickens infected with a chicken astrovirus. Forty one-day-old broiler chickens were infected with either a chicken astrovirus (CkAstV-p5) or were not infected (control). (A) Five birds from each group were euthanized at the given hours post inoculation (h p.i.) and were weighed. (B) The number of cystic lesions and the presence of viral RNA, as detected by ISH, in the duodenal loop from CkAstV-p5 infected birds. Tissue location of the ISH signals was differentiated between the gut villi (“v”) and the crypt region (“c”). (C) The presence of ISH signals were documented as an example for five time points after infection. ISH signals in the dilated cystic lesions were marked with arrows. The inset in the 72 h p.i. represented an H&E stained section and showed a lesion in the crypt region of the duodenum. *Bird with the dilated cystic lesions at 72 h p.i. The ISH score was based on the following scale: 0 no signals; 1=five signals per high-power field; 2=five to 15 signals per high-power field; 3>15 signals per high-power field. Bar=50 μm.

FIG. 4 shows serial passage of a chicken astrovirus in broiler chickens resulted in RSS outcome. Ten one-day-old broiler chickens were infected with either a chicken astrovirus (CkAstV) or were not infected (control). The filtered gut content from the initial chicken astrovirus infected chickens were serially passaged (pass) until passage 5. At day 5 after infection, the body weights, presence of viral RNA as detected by in situ hybridization (ISH), and the number of chickens with cystic lesions in the duodenum were determined. (A) The average weight of each group is shown, as well as the percentage of weight difference between the groups of each passage. *p<0.05. ***p<0.0001. During a subsequent 6th passage, in ten one-day-old broiler chickens per group, gut content of chicken astrovirus infected chickens and mock-infected chickens (cont) from passage 5 either filtered or unfiltered, was used for oral inoculation. Ten chickens were not inoculated and served as additional negative controls (Neg control). (B) The average body weight for each group is shown, and the percentage of differences compared with the negative control group is marked by an asterisk. **p<0.01. (C) The number of birds which showed ISH signals (as a percent of total number of birds) and the number of birds which showed cystic lesions in the duodenal sections after the 6th passage were indicated. (D) Virus titers were determined from each passage material in LMH cells. (E) Examples of cystic lesions in the crypt region of the duodenum during chicken astrovirus passage 1, passage 3, and 6. For comparison, cystic lesions are shown after infection with gut material of RSS affected chickens. Bar=200 μm.

FIG. 5 shows induced RSS outcome by CkAstV backpassage was independent of the passage of gut flora in commercial broiler chickens. (A) A schematic diagram of the passages (pass) of gut content (GC) obtained from broiler chickens infected with chicken astrovirus (Ck-AstVp5) or mock-infected with cell culture medium (CC-medium). At day 5 p.i., duodenal loop homogenates from each of the passage 1 groups (i.e., CkAstV-p5-Ckp1 and negative control-p1) were prepared. The gut content was either filtered (GCF) or left unfiltered (GCUF) prior inoculation for the subsequent passage. For the second passage, four groups of chickens were placed and inoculated orally as follows: group 1: filtered gut homogenate from the negative control of passage 1, group 2: unfiltered gut homogenate from the negative control of passage 1, group 3: filtered gut homogenate from CkAstV-p5-Ckp1) passage 1, and group 4: unfiltered gut homogenate from CkAstV-p5-Ckp1 passage 1. On day 5 p.i. of passage 2, the intestinal samples were collected from each group and processed by group treatment. In detail, intestines from birds inoculated with filtered homogenate were processed to obtain a filtered homogenate, while intestines from the unfiltered homogenate were prepared as unfiltered homogenate. Accordingly, continued passage of these four groups as described for the first passage was performed until passage 5. (B) The average body weights of each group of chickens at day 5 after inoculation. For the first passage (pass 1) the chickens were inoculated either with cell culture medium or with CkAstV-p5. The subsequent passage groups (passage 2 to 5) included the passage of filtered and unfiltered gut content from chickens which have been initially inoculated with cell culture medium (Cont filt, Cont unfilt). The remaining groups were used to passage the filtered and unfiltered gut content from chickens which initially have been infected with CkAstV-p5 (CkAstV filt, CkAstV unfilt). *p<0.05, **p<001. (C) The virus titers were determined from each passage material in LMH cells. (D) The presence of viral RNA as detected by in situ hybridization (ISH) was determined from 5th passage at day 5 p.i. (E) The ISH score was based on the following scale: 0 no signals; 1=five signals per high-power field; 2=five to 15 signals per high-power field; 3>15 signals per high-power field. The average body weight for each group after a subsequent additional passage (6th passage). Filtered gut content of chicken astrovirus infected chickens from passage 5 was used for oral inoculation with serial 10-fold dilutions. *p<0.05. **p<0.01. (F) The presence of viral RNA was determined by ISH. The ISH score was based on the following scale: 0 no signals; 1=five signals per high-power field; 2=five to 15 signals per high-power field; 3>15 signals per high-power field.

FIG. 6 shows a comparison of the genomic architecture of chicken astroviruses and of the amino acid sequence differences. The numbering is in accordance with the full length sequence of a chicken astrovirus from the gut of RSS-affected chickens (NCBI Genbank accession number JF414802). (A) The nucleotide size difference of the nonstructural polyprotein (ORF1a), the RNA dependent RNA polymerase (ORF1b), and the capsid protein (ORF2; NCBI Genbank accession number JF414802) and a chicken astrovirus which has been isolated in cell culture (cc), and the same virus following five passages in chickens (CkAstV-p5-Ckp5). Each noncoding region (NCR) and each open reading frame (ORF) are distinguished and indicated by the size (not drawn to scale). (B) The comparison of ORF1a isolated from chickens (gut-ORF1a; SEQ ID NO:23) and isolated in cell culture (cc-ORF1a; SEQ ID NO:24). (C) The comparison of ORF1b isolated from chickens (gut-ORF1b; SEQ ID NO:25) and isolated in cell culture (cc-ORF1b; SEQ ID NO:26). (D) The comparison of ORF2 isolated from chickens (gut-ORF2; SEQ ID NO:27) and isolated from cell culture (cc-ORF2; SEQ ID NO:28). In B-D, due to the length of the sequences only amino acid sequences were shown which are different except for the first (methionine) and last amino acid of each protein. Dashes represent single identical amino acids while dots represent stretches of identical amino acid sequences of varying lengths. Asterisks represent amino acids not present in the corresponding sequence. (E) Positions of amino acid differences of CkAstV-Gut and CkAstV-p5 are marked in black and positions of deletions in gray on the line which indicates the open reading frame backbone for ORF1a, ORF1b, and ORF2. (F) The amino acid sequences of CkAstV-p5 (cc) and the same virus following five passages in chickens (CkAstV-p5-Ckp5) were compared. Below the diagrams, different sequences in ORF1a and ORF2 between CkAstV-p5 and CkAstV-p5-Ckp5 were shown in comparison with the sequences of CkAstV-Gut. Only sequences which were different in the nonstructural protein (ORF1a; SEQ ID NO:29) and capsid protein (ORF2) were shown. Identical amino acid sequences were marked by dashes.

FIG. 7. Conserved nucleotides in the noncoding regions of bird astroviruses. The 50- and 30-noncoding regions (NCR) of the chicken astrovirus JF414802, as described herein), turkey astrovirus 1 (Jonassen et al., 1998, J Gen Virol; 79:715-718), turkey astrovirus 2 (Strain et al., 2008, J Virol; 82:5099-5103), duck astrovirus (Fu et al., 2009, J Gen Virol; 90:1104-1108), and avian nephritis virus 1 (ANV1, (Imada et al., 2000, J Virol; 74:8487-8493)) were aligned. Highly conserved nucleotides in the 50-NCR are marked by an asterisk. The highly conserved nucleotides in the 30-NCR between chicken astrovirus, both turkey astroviruses, and the duck astrovirus are marked by an asterisk, while the highly conserved nucleotides between all analyzed sequences of the 30-NCR are marked by a plus sign. The poly-A sequence at the 30-NCR was labeled as (A)n.

FIG. 8. Schematic of the genomic organization of the chicken astrovirus. (A) The position of the open reading frames encoding for the nonstructural (NS) polyprotein, RNA-dependent RNA polymerase (RdRp), and the capsid protein (Capsid) are shown. The viral RNA associated poly-A tail [(A)n] is shown. (B) The heptanucleotide sequences (chicken and duck astrovirus) and octanucleotide sequences (turkey astrovirus 1 and 2) serving as the proposed “shifty” sequence as part of the potential ribosomal frameshift signal (Jiang et al., 1993, Proc Natl Acad Sci USA; 90:10539-10543) are highlighted by bold type letters. The noncoding region for the NS protein is marked by an asterisk. The location of the methionine marked in single letter code likely serving as start amino acid for the RdRP of the novel chicken astrovirus, is highlighted bold typed. (C) The secondary structure for the proposed region of the potential ribosomal frameshift signal is shown for the chicken astrovirus (JF414802) and human astrovirus 2 (L13745). The heptanucleotide sequence is highlighted by asterisks and the proposed hairpin structure (Chicken Astrovirus) and stem-loop structure (Human Astrovirus 2) are marked by a bracket. The nucleotide numbers shown in the structures is in accordance to the numbering in the sequence published in the Genbank.

FIG. 9. The sequence of the novel chicken astrovirus forms a new branch. The full length sequences of published full length sequences of turkey astrovirus (TkAstV) 1 and 2, duck astrovirus (DkAstV), chicken astrovirus (CkAstV), avian nephritis virus 1 (ANV1), bat astrovirus (BatAstV), human astrovirus (HumAstV), mink astrovirus MkAstV), and ovine astrovirus (OvAstV). The NCBI Genbank accession number was shown in brackets. The phylogenic tree of a neighbor-joining method is shown performed with 1000 replications. The bootstrap values are shown at the branch knobs.

### DETAILED DESCRIPTION OF ILLUSTRATIVE EMBODIMENTS

Runting and stunting syndrome (RSS) in chickens is a transmissible disease of uncertain etiology. RSS affects chickens early in life and is characterized by growth retardation, ruffled feathers, and diarrhea resulting in considerable economic losses especially in commercial broiler production. The syndrome is also known as malabsorption syndrome, infectious stunting syndrome, broiler runting syndrome, and helicopter syndrome (Rebel et al., 2006, World Poultry Sci J; 62:17-30). Currently, there is no effective licensed vaccine against the disease, mainly because of the absence of known etiologic agents.

The present invention includes the isolation of chicken astroviruses associated with RSS in chickens. The full length genomic nucleotide sequence was obtained from three isolates; a chicken astrovirus isolated from the intestinal content of RSS affected chickens (“CkAstV-gut”) (SEQ ID NO:1); a chicken astrovirus isolated in cell culture (“CkAstV-p5”) (SEQ ID NO:2); and a chicken astrovirus after back passage in chicken (“CKAstV-p5-CK5”) (SEQ ID NO:3). The pathogenesis of these astrovirus isolates was evaluated for ability to induce clinical signs and microscopic lesions of RSS in commercial broiler chickens.

Astroviruses are small, round, non-enveloped viruses with a positive-sense, single-stranded RNA genome, and belong to the virus family, Astroviridae. Virus particles, 28-30 nm in diameter with a star-like shape, can be observed by electron microscopy. Astroviruses have been isolated from feces in a wide variety of animals (e.g. including humans, cats, cattle, deer, dogs, ducks, mice, pigs, sheep, mink, turkeys, chickens, bats, cheetahs, guinea fowl, rats and marine mammals) and the identification was mostly associated with gastroenteritis in young individuals. The genome length varies between 6.8 and 7.9 kb irrespective of the species of isolation.

The genome encodes for three proteins, the nonstructural polyprotein (NS polyprotein), the RNA dependent RNA polymerase (RdRp), and the capsid protein (Jiang et al., 1993, Proc Natl Acad Sci USA; 90:10539-10543). The NS polyprotein and the capsid protein are each encoded by an individual open reading frame (ORF), ORF1a and ORF2, while the RdRP (ORF1b) has been reported to be expressed via a ribosome shift mechanism (Jiang et al., 1993, Proc Natl Acad Sci USA; 90:10539-10543) as a fusion protein to the NS protein (Marczinke et al., 1994, J Virol; 68:5588-5595).

The present invention includes a novel chicken astrovirus (CkAstV) isolated from gut contents of chickens affected with the runting stunting syndrome, also referred to herein as CkAstV-Gut, and its full length genomic nucleotide sequence determined. In some aspects, a chicken astrovirus of the present invention may have a full length genomic sequence having at least about 75% sequence identity, at least about 80% sequence identity, at least about 85% sequence identity, at least about 90% sequence identity, at least about 95% sequence identity, at least about 98% sequence identity, or at least about 99% sequence identity to the genomic sequence of the CkAstV-Gut (SEQ ID NO:1) and attenuations, derivatives, tuncations, or fragments thereof. In some aspects, a chicken astrovirus of the present invention may have a full length genomic sequence SEQ ID NO:1. In some embodiments, the isolated chicken astrovirus includes at least one nucleotide substitution modification relative to SEQ ID NO:1.

In some aspects, a chicken astrovirus of the present invention has an open reading frame 1a (ORF1a) with an amino acid sequence having at least about 75% sequence identity, at least about 80% sequence identity, at least about 85% sequence identity, at least about 90% sequence identity, at least about 95% sequence identity, at least about 96% sequence identity, at least about 97% sequence identity, at least about 98% sequence identity, or at least about 99% sequence identity to SEQ ID NO:4 and attenuations and derivatives thereof. In some aspects, a chicken astrovirus of the present invention has an open reading frame 1a (ORF1a) with amino acid sequence SEQ ID NO:4. In some embodiments, the isolated chicken astrovirus includes an ORF1a with at least one amino acid substitution modification relative to SEQ ID NO:4.

In some aspects, a chicken astrovirus of the present invention has an open reading frame 1b (ORF1b) with an amino acid sequence with at least about at least about 75% sequence identity, at least about 80% sequence identity, at least about 85% sequence identity, at least about 90% sequence identity, at least about 95% sequence identity, at least about 96% sequence identity, at least about 97% sequence identity, at least about 98% sequence identity, or at least about 99% sequence identity to SEQ ID NO:7, and attenuations and derivatives thereof. In some aspects, a chicken astrovirus of the present invention has an open reading frame 1b (ORF1b) with amino acid sequence SEQ ID NO:7. In some embodiments, the isolated chicken astrovirus includes an ORF1b with at least one amino acid substitution modification relative to SEQ ID NO:7.

In some aspects, a chicken astrovirus of the present invention has an open reading frame 2 (ORF2) with an amino acid sequence with at least about 75% sequence identity, at least about 80% sequence identity, at least about 85% sequence identity, at least about 90% sequence identity, at least about 95% sequence identity, at least about 96% sequence identity, at least about 97% sequence identity, at least about 98% sequence identity, or at least about 99% sequence identity SEQ ID NO:10, and attenuations and derivatives thereof. In some aspects, a chicken astrovirus of the present invention has an open reading frame 2 (ORF2) with amino acid sequence SEQ ID NO:10. In some embodiments, the isolated chicken astrovirus includes an ORF2 with at least one amino acid substitution modification relative to SEQ ID NO:10.

Further, the present invention includes a cell culture adapted astrovirus (ccCkAStV). Such a cell culture adapted chicken astrovirus may be obtained, for example, with one or more passage in cell culture, two or more passages in cell culture, three or more passages in cell culture, four or more passages in cell culture, five or more passages in cell culture, or six or more passages in cell culture. Such a cell culture adapted chicken astrovirus may be obtained, for example, with about one passage in cell culture, about two passages in cell culture, about three passages in cell culture, about four passages in cell culture, about five passages in cell culture, about six passages in cell culture, or any range thereof. Examples include a cell culture chicken astroviruses described herein, obtained after one passage (CkAstV-p1), two passages (CkAstV-p2), three passages (CkAstV-p3), four passages (CkAstV-p4), or five passages (CkAstV-p5). In some embodiments, the cell culture chicken astroviruses is CkAstV-p5.

Cell lines that may be used include, but are not limited to, Madin Darby canine kidney cells (MDCK, CRL-2285, ATCC, Manassas, Va.), DF1, a chicken fibroblastoid cell line (CRL-12203, ATCC), Vero cells (CRL-1587; ATCC), and LMH, a chicken hepatocellular carcinoma epithelial cell line (CRL-2117, ATCC).

In some aspects, a chicken astrovirus of the present invention has a full length genomic sequence with at least about 75% sequence identity, at least about 80% sequence identity, at least about 85% sequence identity, at least about 90% sequence identity, at least about 95% sequence identity, at least about 98% sequence identity, or at least about 99% sequence identity to the genomic sequence of the cell culture chicken astrovirus CkAstV-5p (SEQ ID NO:2) and attenuations, derivatives, tuncations, or fragments thereof. In some aspects, a chicken astrovirus of the present invention has a full length genomic sequence SEQ ID NO:2. In some embodiments, the isolated chicken astrovirus includes at least one nucleotide substitution modification relative to SEQ ID NO:1 and/or SEQ ID NO:2.

In some aspects, a chicken astrovirus of the present invention has an open reading frame 1a (ORF1a) with an amino acid sequence having at least about 75% sequence identity, at least about 80% sequence identity, at least about 85% sequence identity, at least about 90% sequence identity, at least about 95% sequence identity, at least about 96% sequence identity, at least about 97% sequence identity, at least about 98% sequence identity, or at least about 99% sequence identity to the ORF1a of the cell culture chicken astrovirus CkAstV-5p (SEQ ID NO:5) and attenuations and derivatives thereof. In some aspects, a chicken astrovirus of the present invention has an open reading frame 1a (ORF1a) with amino acid sequence SEQ ID NO:5. In some embodiments, the isolated chicken astrovirus includes an ORF1a with at least one amino acid substitution modification relative to SEQ ID NO:4 and/or SEQ ID NO:5.

In some aspects, a chicken astrovirus of the present invention has an open reading frame 1b (ORF1b) with an amino acid sequence with at least about at least about 75% sequence identity, at least about 80% sequence identity, at least about 85% sequence identity, at least about 90% sequence identity, at least about 95% sequence identity, at least about 96% sequence identity, at least about 97% sequence identity, at least about 98% sequence identity, or at least about 99% sequence identity to the ORF1b of the cell culture chicken astrovirus CkAstV-5p (SEQ ID NO:8) and attenuations and derivatives thereof. In some aspects, a chicken astrovirus of the present invention has an open reading frame 1b (ORF1b) with amino acid sequence SEQ ID NO:8. In some embodiments, the isolated chicken astrovirus includes an ORF1a with at least one amino acid substitution modification relative to SEQ ID NO:7 and/or SEQ ID NO:8.

In some aspects, a chicken astrovirus of the present invention has an open reading frame 2 (ORF2) with an amino acid sequence with at least about 75% sequence identity, at least about 80% sequence identity, at least about 85% sequence identity, at least about 90% sequence identity, at least about 95% sequence identity, at least about 96% sequence identity, at least about 97% sequence identity, at least about 98% sequence identity, or at least about 99% sequence identity to the ORF2 of the cell culture chicken astrovirus CkAstV-5p (SEQ ID NO:11), and attenuations and derivatives thereof. In some aspects, a chicken astrovirus of the present invention has an open reading frame 2 (ORF2) with amino acid sequence SEQ ID NO:11. In some embodiments, the isolated chicken astrovirus includes an ORF1a with at least one amino acid substitution modification relative to SEQ ID NO:10 and/or SEQ ID NO:11.

Further, the present invention includes cell culture adapted astrovirus back passaged in chickens. Such a back passaged chicken astrovirus may be obtained, for example, with one or more back passages, two or more back passages, three or more back passages, four or more back passages, five or more back passages, or six or more back passages. Such a back passaged chicken astrovirus may be obtained, for example, with about one back passages, about two back passages, about three back passages, about four back passages, about five back passages, about six back passages, or any range thereof. Examples, include CkAstV-p5-Ck1 (one back passage), CkAstV-p5-Ck2 (two back passages), CkAstV-p5-Ck3 (three back passages), CkAstV-p5-Ck4 (four back passages), CkAstV-p5-Ck5 (five back passages), and CkAstV-p5-Ck6 (six back passages), as described herein. In some embodiments, the cell culture chicken astroviruses is CkAstV-p5-Ck5.

In some aspects, a chicken astrovirus of the present invention has a full length genomic sequence with at least about 75% sequence identity, at least about 80% sequence identity, at least about 85% sequence identity, at least about 90% sequence identity, at least about 95% sequence identity, at least about 98% sequence identity, or at least about 99% sequence identity to the genomic sequence of the chicken astrovirus CkAstV-5p-Ckp5 (SEQ ID NO:3), and attenuations, derivatives, tuncations, or fragments thereof. In some aspects, a chicken astrovirus of the present invention has a full length genomic sequence SEQ ID NO:3. In some embodiments, the isolated chicken astrovirus includes at least one nucleotide substitution modification relative to SEQ ID NO:1 and/or SEQ ID NO:3.

In some aspects, a chicken astrovirus of the present invention has an open reading frame 1a (ORF1a) with an amino acid sequence having at least about 75% sequence identity, at least about 80% sequence identity, at least about 85% sequence identity, at least about 90% sequence identity, at least about 95% sequence identity, at least about 96% sequence identity, at least about 97% sequence identity, at least about 98% sequence identity, or at least about 99% sequence identity to the ORF1a of the chicken astrovirus CkAstV-5p-Ckp5 (SEQ ID NO:6) and attenuations and derivatives thereof. In some aspects, a chicken astrovirus of the present invention has an open reading frame 1a (ORF1a) with amino acid sequence SEQ ID NO:6. In some embodiments, the isolated chicken astrovirus includes an ORF1a with at least one amino acid substitution modification relative to SEQ ID NO:4 and/or SEQ ID NO:6.

In some aspects, a chicken astrovirus of the present invention has an open reading frame 1b (ORF1b) with an amino acid sequence with at least about at least about 75% sequence identity, at least about 80% sequence identity, at least about 85% sequence identity, at least about 90% sequence identity, at least about 95% sequence identity, at least about 96% sequence identity, at least about 97% sequence identity, at least about 98% sequence identity, or at least about 99% sequence identity to the ORF1a of the chicken astrovirus CkAstV-5p-Ckp5 (SEQ ID NO:9), and attenuations and derivatives thereof. In some aspects, a chicken astrovirus of the present invention has an open reading frame 1b (ORF1b) with amino acid sequence SEQ ID NO:9. In some embodiments, the isolated chicken astrovirus includes an ORF1b with at least one amino acid substitution modification relative to SEQ ID NO:7 and/or SEQ ID NO:9.

In some aspects, a chicken astrovirus of the present invention has an open reading frame 2 (ORF2) with an amino acid sequence with at least about 75% sequence identity, at least about 80% sequence identity, at least about 85% sequence identity, at least about 90% sequence identity, at least about 95% sequence identity, at least about 96% sequence identity, at least about 97% sequence identity, at least about 98% sequence identity, or at least about 99% sequence identity the ORF1a of the chicken astrovirus CkAstV-5p-Ckp5 (SEQ ID NO:12), and attenuations and derivatives thereof. In some aspects, a chicken astrovirus of the present invention has an open reading frame 2 (ORF2) with amino acid sequence SEQ ID NO:12. In some embodiments, the isolated chicken astrovirus includes an ORF2 with at least one amino acid substitution modification relative to SEQ ID NO:10 and/or SEQ ID NO:12.

In some aspects, a chicken astrovirus of the present invention has an alanine (A) at amino acid 6 of ORF1a. In some aspects, a chicken astrovirus of the present invention has a valine (V) at amino acid 6 of ORF1a.

In some aspects, a chicken astrovirus of the present invention has a valine (V) at amino acid 45 of ORF1a. In some aspects, a chicken astrovirus of the present invention has an alanine (A) at amino acid 6 of ORF1a.

In some aspects, a chicken astrovirus of the present invention has a serine (S) at amino acid 50 of ORF1a. In some aspects, a chicken astrovirus of the present invention has a threonine (T) at amino acid 50 of ORF1a.

In some aspects, a chicken astrovirus of the present invention has a phenylalanine (F) at amino acid 371 of ORF2. In some aspects, a chicken astrovirus of the present invention has a tyrosine (Y) at amino acid 371 of ORF2.

An isolated chicken astrovirus as described herein may demonstrate reduced virulence and serve as an attenuated virus for vaccination purposes. Such attenuation may include any one or more of the properties described in the examples section included herewith.

The present invention also includes cell lines infected with a virus as described herein and cell lines adapted for the culture/passage/replication of a virus as described herein. Such a cell line may be a cell line of avian origin. Such a cell line may be a cell line of chicken origin. Examples include, but are not limited to, the MDCK, DF1, QM7, Vero, or LMH cell lines. In some embodiments, the cell line is LMH, a chicken hepatocellular carcinoma epithelial cell line (CRL-2117, ATCC). In some aspects, the LMH cell line has been adapted for growth at approximately 39° C.

The present invention as includes in vitro methods of culturing an astrovirus, including, but not limited to a chicken astrovirus as described herein. Such a method may include any one or more of the steps of the cell culture methods described in the examples section included herein with. In some aspects, the method may include culturing of an astrovirus in a cell line of avian origin. Such a cell line may be a cell line of chicken origin, such as, for example, the MDCK, DF1, QM7, Vero, or LMH cell lines. In some aspects, the cell line is LMH, a chicken hepatocellular carcinoma epithelial cell line (CRL-2117, ATCC). In some aspects, the LMH cell line has been adapted for growth at approximately 39° C. In some aspects, the method may include culturing cells and/or virus at about 39° C.

The present invention also includes isolated supernatants or cellular components obtained from a cell line infected or passaging a virus as described herein. Cell culture supernatants and cellular components may be obtained, for example, after centrifugation or filtration of a culture of a cell line. In some aspects, replication kinetics of a CkAstV virus as described herein indicate that infectious virus may not be efficiently released into the cell culture supernatant, with more virus particles remaining cell associated compared to virus present in the supernatant.

In some aspects of the present invention, a procedures, methods, compositions, vaccines, capsid nucleotide sequences, and/or capsid amino acid sequence as described in WO 2010/059899 and U.S. patent application Ser. No. 13/107,140 (both of which are herein incorporated by reference in their entireties) may be used.

In some aspects, a virus as described herein, or cell line infected with such a virus, may be put on deposit with the American Type Culture Collection (ATCC®), 10801 University Boulevard, Manassas, Va. 20110-2209, USA, as PTA-9495 on Sep. 15, 2008. Such a deposit may be in accordance with the Budapest Treaty on the International Recognition of the Deposit of Microorganisms for the Purposes of Patent Procedure.

The present invention includes isolated viruses, polypeptides, polynucleotides, and antibodies. As used herein, “isolated” refers to material removed from its original environment (e.g., the natural environment if it is naturally occurring), and thus is altered “by the hand of man” from its natural state.

The present invention includes polynucleotide sequences that encode the viral genomes, open reading frames and various polypeptides described herein, truncations, or fragments thereof.

The present invention includes isolated polynucleotide sequences with at least about 60% sequence identity, at least about 65% sequence identity, at least about 70% sequence identity, at least about 75% sequence identity, at least about 80% sequence identity, at least about 85% sequence identity, at least about 90% sequence identity, at least about 95% sequence identity, at least about 96% sequence identity, at least about 97% sequence identity, at least about 98% sequence identity, or at least about 99% sequence identity to a viral genome sequences represented by the nucleotide sequence of SEQ ID NO:1, SEQ ID NO:2, or SEQ ID NO:3 and the polynucleotide sequences encoding an open reading frames of SEQ ID NO:4-12.

The present invention includes an isolated polynucleotide sequence having the nucleotide sequence of SEQ ID NO:1, SEQ ID NO:2, or SEQ ID NO:3 or a polynucleotide sequences encoding the open reading frames of SEQ ID NO:4-12. In some embodiments, the polynucleotide sequence includes at least one nucleotides substitution modification relative to SEQ ID NO:1, SEQ ID NO:2, SEQ ID NO:3, and/or a polynucleotide sequence encoding an open reading frames of SEQ ID NO:4-12.

The present invention includes polynucleotide sequences that hybridize to a nucleotide sequence described herein (such as, for example a viral genome sequences represented by SEQ ID NO:1, SEQ ID NO:2, or SEQ ID NO:3 or the polynucleotide sequences encoding the open reading frames of SEQ ID NO:4-12) under various stringency conditions, and fragments thereof. Stringency conditions include, but are not limited to, moderate and high stringency. High stringency hybridization conditions may be, for example, 6×SSC, 5×Denhardt, 0.5% sodium dodecyl sulfate (SDS), and 100 μg/ml fragmented and denatured salmon sperm DNA hybridized overnight at 65° C. and washed in 2×SSC, 0.1% SDS at least one time at room temperature for about 10 minutes followed by at least one wash at 65° C. for about 15 minutes followed by at least one wash in 0.2×SSC, 0.1% SDS at room temperature for at least 3 to 5 minutes.

The present invention includes a polynucleotide sequence described herein having a substitution of one, two, three, four, five, six, seven, eight, nine, ten, eleven, twelve, thirteen, fourteen fifteen, twenty, or more nucleotides. The present invention also includes the polynucleotide sequences described herein in which codon usage has been adapted to optimize expression in a given host cell. For example, codon usage may be adapted to optimize for expression in host cells including, but not limited to, baculovirus, yeast, E. coli, poultry, or human cells. Such adaptation can be carried out by techniques know in the art.

The present invention includes primers, including, but not limited to, any of the primers described herein, and primers that can be used to generate a sequence described herein, or a fragment thereof, in a PCR reaction. In some embodiments, a primer may include at least 5, at least 10, at least 15, at least 20, at least 25, at least 30, at least 35, or at least 40 nucleotide residues. In some embodiments, a primer may include no more than 10, no more than 15, no more than 20, no more than 25, no more than 30, no more than 35, no more than 40, no more than 45, no more than 50, no more than 55, or no more than 60 nucleotide residues. Such nucleotides residues may be consecutive sequences or its complement. Also included are primer pairs including at least one of the primers described herein, complements thereof, or primers derived from such sequences. Also included in the present invention are the amplification products produced by such primers.

The present invention provides a recombinant vector containing one or more of the nucleotide sequences described herein. Such a recombinant vector may be an expression vector. Such a recombinant vector may also include other sequences such as expression control sequences, markers, amplifying genes, signal sequences, promoters, and the like, as is known in the art. Useful vectors for this purpose are plasmids, and viruses such as baculoviruses, paramyxovirus, coronavirus, herpes virus (for example, herpes virus of turkeys (HVT)) and pox viruses, for example, fowl pox virus, and the like.

In some aspects, a vector is a vaccine vector. One example of such a vaccine vector is a Newcastle disease based vector, as described, for example, by Estevez et al., 2007, Virus Research; 129:182-190; Susta et al., 2010, Tropical Animal Health and Production; 42(8):1785-1795; Hu et al., 2011, Vaccine; 29(47):8624-33; Hu et al., 2012, China Poultry; 34(12):1-5; and Li et al., 2012, Virology Journal; 9:227, each of which is incorporated by reference herein in its entirety.

The present invention also includes host cells transformed with a polynucleotide sequence described herein and host cells transformed with a recombinant vector described herein. The host cell may be, for example, a eukaryotic or a prokaryotic host cell. Suitable examples are E. coli, insect cell lines such as Sf-9, chicken embryo fibroblast (CEF) cells, chicken embryo kidney (CEK) cells, African green monkey Vero cells and the like.

The present invention includes polynucleotides sequences, viruses, and polypeptides as described herein, truncations and fragments thereof. Truncations include, but are not limited to, amino acid sequences in which one, two, three, four, five, six, seven, eight, nine, ten, or more amino acids are removed from the amino terminus of an amino acid sequence and/or one, two, three, four, five, six, seven, eight, nine, ten, or more amino acids are removed from the carboxy terminus of an amino acid sequence.

Fragments include, but are not limited to, for example, fragments having about 5, about 10, about 15, about 20, about 25, about 50, about 75, about 100, about 150, about 200, about 250, about 300, about 350, about 400, about 450, about 500, about 550, about 600, about 650, and about 700 consecutive amino acid residues of a sequence described herein. Fragments also include, for example, fragments of a size range of any combination of the above fragment sizes.

Fragments include, but are not limited to, for example, fragments having at least 5, at least 10, at least 15, at least 20, at least 25, at least 50, at least 75, at least 100, at least 150, at least 200, at least 250, at least 300, at least 350, at least 400, at least 450, at least 500, at least 550, at least 600, at least 650, and at least 700 consecutive amino acid residues of a sequence described herein.

The present invention includes polypeptides having an amino acid sequence with at least about 75% sequence identity, at least about 80% sequence identity, at least about 85% sequence identity, at least about 90% sequence identity, at least about 95% sequence identity, at least about 96% sequence identity, at least about 97% sequence identity, at least about 98% sequence identity, or at least about 99% sequence identity to an amino acid sequence of any one of the open reading frames of SEQ ID NO:4-12, or a polypeptide encoded by a viral genome sequence represented by SEQ ID NO:1, SEQ ID NO:2, or SEQ ID NO:3. The present invention includes polypeptides having an amino acid sequence of any one of the open reading frames of SEQ ID NO:4-12, or a polypeptide encoded by a viral genome sequence represented by SEQ ID NO:1, SEQ ID NO:2, or SEQ ID NO:3. In some embodiments, a polypeptide includes at least one amino acid substitution modification relative to an ORF of any one of SEQ ID NO:4-12.

The present invention includes polypeptides having an amino acid sequence with one, two, three, four, five, six, seven, eight, nine, ten, eleven, twelve, thirteen, fourteen, fifteen, sixteen, seventeen, eighteen, nineteen, twenty, or more amino acid changes from an amino acid sequence described herein, or fragments thereof. Such amino acid changes include, but are not limited to, conservative amino acid changes.

The present invention includes polypeptides encoded by a polynucleotide that hybridizes to the nucleotide sequence described herein under stringent hybridization conditions, and fragments thereof. Stringent hybridization conditions may be, for example, 6×SSC, 5×Denhardt, 0.5% sodium dodecyl sulfate (SDS), and 100 μg/ml fragmented and denatured salmon sperm DNA hybridized overnight at 65° C. and washed in 2×SSC, 0.1% SDS at least one time at room temperature for about 10 minutes followed by at least one wash at 65° C. for about 15 minutes followed by at least one wash in 0.2×SSC, 0.1% SDS at room temperature for at least 3 to 5 minutes. Such polypeptides may be bound by an antibody that specifically binds to a polypeptide as described herein, or a fragment thereof.

Also included in the present invention are compositions including one or more of the isolated viruses, infected cell lines, cell pellets, cell culture supernatants, polynucleotide sequences, vectors, and/or polypeptides described herein.

Such a composition may include pharmaceutically acceptable carriers or diluents. Carriers include, for example, stabilizers, preservatives and buffers. Suitable stabilizers include, for example, SPGA, carbohydrates (such as sorbitol, mannitol, starch, sucrose, dextran, glutamate or glucose), proteins (such as dried milk serum, albumin or casein) or degradation products thereof. Suitable buffers include, for example, alkali metal phosphates. Suitable preservatives include, for example, thimerosal, merthiolate and gentamicin. Diluents, include, but are not limited to, water, aqueous buffer (such as buffered saline), alcohols, and polyols (such as glycerol).

The present invention includes immunogenic compositions and vaccines including one or more of the isolated viruses, infected cell lines, cell pellets, cell culture supernatants, polynucleotide sequences, vectors, and/or polypeptides described herein. In some embodiments, the viruses, infected cell lines, cell pellets, or cell culture supernatants are live. In some embodiments, the viruses, infected cell lines, cell pellets, or cell culture supernatants are attenuated or inactivated. In some embodiments, the viruses, infected cell lines, cell pellets, or cell culture supernatants are killed. In some embodiments, the organisms, compositions, or vaccines may be lyophilized.

Such a compositions and vaccine may be administered as the active component to immunize a bird to elicit an antibody response to RSS and/or induce immunity against RSS. Immunity may include the induction of a significant higher level of protection in a population of birds after vaccination compared to an unvaccinated group.

An immunogenic composition or vaccine of the present invention may also include one or more compounds with adjuvant activity. Suitable compounds or compositions for this purpose include aluminum hydroxide, aluminum phosphate, aluminum oxide, plant oils, animal oils, oil-in-water or water-in-oil emulsion based on, for example a mineral oil, such as Bayol F™ or Marcol 52™, Complete Freund's adjuvant, incomplete Freund's adjuvant, or a vegetable oil such as vitamin E acetate, and saponins.

An immunogenic composition or vaccine of the present invention may also contain one or more stabilizers. Any suitable stabilizer can be used including carbohydrates such as sorbitol, mannitol, starch, sucrose, dextrin, or glucose; proteins such as albumin or casein; and buffers such as alkaline metal phosphate and the like. A stabilizer is particularly advantageous when a dry vaccine preparation is prepared by lyophilization.

An immunogenic composition or vaccine of the present invention may further include one or more immunogens derived from other pathogens infectious to poultry. Such immunogens may be derived from, for example, Marek's disease virus (MDV), infectious bronchitis virus (IBV), Newcastle disease virus (NDV), egg drop syndrome (EDS) virus, turkey rhinotracheitis virus (TRTV), poxvirus, reovirus, chicken parvovirus, and avian nephritis virus (including, but not limited to ANV-1 and ANV-2).

An immunogenic composition or vaccine of the present invention may be administered by any suitable known method of inoculating poultry including nasally, ophthalmically, by injection, in drinking water, in the feed, by exposure, in ovo, maternally, by respiratory inhalation, and the like. The immunogenic composition or vaccine may be administered by mass administration techniques such as by placing the vaccine in drinking water or by spraying the environment. When administered by injection, the immunogenic composition or vaccine may be administered parenterally. Parenteral administration includes, for example, administration by intravenous, subcutaneous, intramuscular, or intraperitoneal injection.

Compositions and vaccines of the present invention may be substantially pure. As used herein, “substantially pure” will mean material essentially free of any similar macromolecules or other biological entities that would normally be found with it in nature.

Compositions and vaccines of the present invention may be administered to birds of any of a variety of avian species that are susceptible to RSS, including, but not limited to, poultry, birds of the order Galliformes, and exotic bird species. Birds of the order Galliformes include, but are not limited to, chickens, turkeys, grouse, quails, and pheasants. As used herein, poultry includes domesticated birds that are kept for the purpose of collecting their eggs, or killing for their meat and/or feathers. These most typically are members of the superorder Galloanserae (fowl), especially the order Galliformes (which includes, for example, chickens, quail, turkeys, and grouse) and the family Anatidae (in order Anseriformes), commonly known as “waterfowl” (including, for example, ducks, geese, and swans). Poultry may also include other birds which are killed for their meat, such as pigeons or doves or birds considered to be game, like pheasants. Chickens include, but are not limited to, hens, roosters, broilers, roasters, layers, breeders, the offspring of breeder hens, and layers. As used herein, the term “susceptible to” means the possibility or actuality of a detrimental response to the referenced microorganism, such as, for example, reduced vigor or a failure to thrive, when compared to a non-susceptible individuals or groups, and/or one or more pathological state(s) indicative of Runting Stunting Syndrome.

The vaccine of the present invention may be administered to poultry before or after hatching. Poultry may receive a vaccine at a variety of ages. For example, broilers may be vaccinated in ovo, at one-day-old, or at 2-3 weeks of age. Laying stock or reproduction stock may be vaccinated, for example, at about 6-12 weeks of age and boosted at about 16-20 weeks of age. Such laying stock or reproduction stock may be vaccinated at about 6, at about 7, at about 8, at about 9, at about 10, at about 11, or at about 12 weeks of age. Such laying stock or reproduction stock may be boosted at about 16, at about 17, at about 18, at about 19, or at about 20 weeks of age. The offspring of such laying stock or reproduction stock may demonstrate an antibody titer to a polypeptide as described herein, which may prevent or mitigate the symptoms of an RSS infection in the offspring. In ovo vaccination may take place, for example, at about 13 days, about 14 days, about 15 days, about 16 days, about 17 days, about 18 days, about 19 days, about 20 days, or at any range thereof.

The present invention includes antibodies that bind to a polypeptide as described herein, and various antibody fragments, also referred to as antigen binding fragments, which include only a portion of an intact antibody, generally including an antigen binding site of the intact antibody and thus retaining the ability to bind antigen. Such an antibody, or antigen binding fragment thereof, may bind to the polypeptide described herein. Such an antibody, or antigen binding fragment thereof, may bind to a polypeptide including at least five, at least ten, at least eleven, at least twelve, at least thirteen, at least fourteen, at least fifteen, at least twenty, at least twenty five, at least thirty, at least forty, at least fifty, at least seventy-five, at least one hundred, at least two hundred, at least three hundred, at least four hundred, at least five hundred, at least six hundred, or at least seven hundred consecutive amino acid residues of a sequence described herein. Such antibodies may be used to detect or isolate an RSS-associated polypeptide or virus from a sample.

Examples of antibody fragments include, for example, Fab, Fab′, Fd, Fd′, Fv, dAB, and F(ab′)2 fragments produced by proteolytic digestion and/or reducing disulfide bridges and fragments produced from an Fab expression library. Antibodies include, but are not limited to, polyclonal antibodies and monoclonal antibodies. The antibodies of the present invention can be of any type (e.g., IgG, IgE, IgM, IgD, IgA and IgY), class (e.g., IgG1, IgG2, IgG3, IgG4, IgA1 and IgA2) or subclass of immunoglobulin molecule. Immunoglobulins can have both heavy and light chains. An array of IgG, IgE, IgM, IgD, IgA, and IgY heavy chains can be paired with a light chain of the kappa or lambda form.

An intact antibody molecule has two heavy (H) chain variable regions (abbreviated herein as VH) and two light (L) chain variable regions (abbreviated herein as VL). The VH and VL regions can be further subdivided into regions of hypervariability, termed “complementarity determining regions” (“CDR”), interspersed with regions that are more conserved, termed “framework regions” (FR). The extent of the framework region and CDR's has been precisely defined. Each VH and VL is composed of three CDR's and four FRs, arranged from amino-terminus to carboxy-terminus in the following order: FR1, CDR1, FR2, CDR2, FR3, CDR3, FR4. The present invention includes an antibody with the heavy chain, the light chain, the heavy chain variable region, the light chain variable region, and/or one or more complementarity determining regions of a monoclonal antibody of the present invention. The present invention includes bispecific or bifunctional antibodies. A bispecific or bifunctional antibody is an artificial hybrid antibody having two different heavy/light chain pairs and two different binding sites. Bispecific antibodies can be produced by a variety of methods including fusion of hybridomas or linking of F(ab′) fragments.

The antibodies of the invention can be from any animal origin, including birds and mammals. In some embodiments, the antibodies are human, murine, rat, donkey, sheep, rabbit, goat, guinea pig, camel, horse, llama, camel, or chicken antibodies. As used herein, “human” antibodies include antibodies having the amino acid sequence of a human immunoglobulin and include antibodies isolated from human immunoglobulin libraries or from animals transgenic for one or more human immunoglobulins.

Monoclonal antibodies of the present invention can be obtained by various techniques familiar to those skilled in the art. For example, spleen cells from an animal immunized with a desired antigen are immortalized, commonly by fusion with a myeloma cell. Monoclonal antibodies can be isolated and purified from hybridoma cultures by techniques well known in the art. Other known methods of producing transformed B cell lines that produce monoclonal antibodies may also be used. In some embodiments, the antibody can be recombinantly produced, for example, produced by phage display or by combinatorial methods. Such methods can be used to generate human monoclonal antibodies.

Also included in the present invention are phage display libraries expressing one or more hypervariable regions from a monoclonal antibody of the present invention, and clones obtained from such a phage display library. A phage display library is used to produce antibody derived molecules. Gene segments encoding the antigen-binding variable domains of antibodies are fused to genes encoding the coat protein of a bacteriophage. Bacteriophage containing such gene fusions are used to infect bacteria, and the resulting phage particles have coats that express the antibody-fusion protein, with the antigen-binding domain displayed on the outside of the bacteriophage. Phage display libraries can be prepared, for example, using the Ph.D.™-7 Phage Display Peptide Library Kit (Catalog No. E8100S) or the Ph.D.™-12 Phage Display Peptide Library Kit (Catalog No. E8110S) available from New England Biolabs Inc., Ipswich, Mass. See also, Smith and Petrenko, 1997, Chem Rev; 97:391-410.

The present invention includes antibodies and binding proteins that include one or more of the complementarity determining regions (CDR) of a monoclonal antibody described herein.

The antibodies of the present invention may be coupled directly or indirectly to a detectable marker by techniques well known in the art. A detectable marker is an agent detectable, for example, by spectroscopic, photochemical, biochemical, immunochemical, or chemical means. Useful detectable markers include, but are not limited to, fluorescent dyes, chemiluminescent compounds, radioisotopes, electron-dense reagents, enzymes, colored particles, biotin, or dioxigenin. A detectable marker often generates a measurable signal, such as radioactivity, fluorescent light, color, or enzyme activity. Antibodies conjugated to detectable agents may be used for diagnostic or therapeutic purposes. Examples of detectable agents include various enzymes, prosthetic groups, fluorescent materials, luminescent materials, bioluminescent materials, radioactive materials, positron emitting metals using various positron emission tomographies, and nonradioactive paramagnetic metal ions. The detectable substance can be coupled or conjugated either directly to the antibody or indirectly, through an intermediate such as, for example, a linker known in the art, using techniques known in the art. See, for example, U.S. Pat. No. 4,741,900, describing the conjugation of metal ions to antibodies for diagnostic use. Examples of suitable enzymes include horseradish peroxidase, alkaline phosphatase, beta-galactosidase, and acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride and phycoerythrin; an example of a luminescent material includes luminol; examples of bioluminescent materials include luciferin, and aequorin; and examples of suitable radioactive material include iodine (¹²¹I, ¹²³I, ¹²⁵I, ¹³¹I), carbon (¹⁴C), sulfur (³⁵S), tritium (³H), indium (¹¹¹In, ¹¹²In, ¹¹³mIn, ¹¹⁵mIn), technetium (⁹⁹Tc, ⁹⁹mTc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, and ⁹⁷Ru. Techniques for conjugating such moieties to antibodies are well-known.

Antibodies of the present invention include derivatives of antibodies that are modified or conjugated by the covalent attachment of any type of molecule to the antibody. Such antibody derivatives include, for example, antibodies that have been modified by glycosylation, acetylation, pegylation, phosphorylation, amidation, derivatization by known protecting/blocking groups, proteolytic cleavage, or linkage to a cellular ligand or other protein. Any of numerous chemical modifications can be carried out by known techniques, including, but not limited to, specific chemical cleavage, acetylation, formylation, and metabolic synthesis of tunicamycin. Additionally, the derivatives can contain one or more non-classical amino acids.

The antibodies of the present invention may “specifically bind to” or be “specific for” a particular polypeptide or an epitope on a particular polypeptide. Such an antibody is one that binds to that particular polypeptide or epitope on a particular polypeptide without substantially binding to any other polypeptide or polypeptide epitope. For example, an antibody may bind to a virus with the genomic sequence of the cell culture chicken astrovirus CkAstV-p5 (SEQ ID NO:2) and/or the genomic sequence the chicken astrovirus after back passage in chicken CkAstV-p5-Ckp5 (SEQ ID NO:3) and not bind to the genomic sequence of the chicken astrovirus isolated from the gut of chickens CkAstV-Gut (SEQ ID NO:1). For example, an antibody may bind to the genomic sequence of the chicken astrovirus isolated from the gut of chickens CkAstV-Gut (SEQ ID NO:1) and not bind to a virus with the genomic sequence of the cell culture chicken astrovirus CkAstV-p5 (SEQ ID NO:2) and/or not bind to a virus with the genomic sequence the chicken astrovirus after back passage in chicken CkAstV-p5-Ckp5 (SEQ ID NO:3). For example, an antibody may bind to one or more of an ORF1a with an amino acid SEQ ID NO:4-6; one or more of an ORF1b with an amino acid sequence SEQ ID NO:7-9, or one or more of an ORF2 with an amino acid sequence SEQ ID NO:10-12. In some aspects, an antibody may bind to one or two of an ORF1a, ORF1b, or ORF2 amino acid sequence and not bind to the remaining ORf1a, ORF1b, or ORF2 amino acid sequence.

Also included in the present invention are hybridoma cell lines, transformed B cell lines, and host cells that produce the monoclonal antibodies of the present invention; the progeny or derivatives of these hybridomas, transformed B cell lines, and host cells; and equivalent or similar hybridomas, transformed B cell lines, and host cells.

The present invention includes kits employing one or more of the viruses, infected cell lines, cell pellets, supernatants, polynucleotide sequences, vectors, polypeptides, and/or antibodies described herein. Such kits may provide for the administration of a virus, infected cell line, cell culture supernatant, cell pellet, polynucleotide, vector, and/or polypeptide of the present invention to an animal in order to elicit an immune response. Such kits may provide for the detection of a virus, polypeptide, antibody or polynucleotide, for example, for the detection of RSS infection or exposure of a subject to an RSS agent. Kits of the present invention may include other reagents such as buffers and solutions needed to practice the invention are also included. Optionally associated with such container(s) can be a notice or printed instructions. As used herein, the phrase “packaging material” refers to one or more physical structures used to house the contents of the kit. The packaging material is constructed by well-known methods, preferably to provide a sterile, contaminant-free environment. As used herein, the term “package” refers to a solid matrix or material such as glass, plastic, paper, foil, and the like, capable of holding within fixed limits a polypeptide. Kits of the present invention may also include instructions for use. Instructions for use typically include a tangible expression describing the reagent concentration or at least one assay method parameter, such as the relative amounts of reagent and sample to be admixed, maintenance time periods for reagent/sample admixtures, temperature, buffer conditions, and the like.

The present invention includes diagnostic and therapeutic reagents and methods for the detection, treatment, and prevention of RSS. The present invention includes a variety of methods employing one or more of the viruses, infected cell lines, cell pellets, supernatants, polynucleotide sequences, vectors, polypeptides, antibodies, compositions, vaccines, and/or host cells described herein.

For example, the viruses, infected cell lines, cell pellets, supernatants, polynucleotide sequences, vectors, polypeptides, antibodies, compositions, vaccines, and/or host cells described herein may be administered to elicit an immune response in poultry or other animals. The immune response may or may not confer protective immunity. An immune response may include, for example, a humoral response and/or a cell mediated response. An immune response may include a mucosal immune response. Such an immune response may result in a reduction or mitigation of the symptoms of future RSS infection. Such an immune response may prevent a future RSS infection in poultry. Such an immune response may be a humoral immune response, a cellular immune response, and/or a mucosal immune response. A humoral immune response may include an IgG, IgM, IgA, IgD, and/or IgE response. The determination of a humoral, cellular, or mucosal immune response may be determined by any of a variety of methods, including, but not limited to, any of those described herein.

The induction of an immune response may include the priming and/or the stimulation of the immune system of poultry to respond to a future challenge with a RSS infectious agent, providing immunity to future RSS infections. The induction of such an immune response may serve as a protective response, generally resulting in a reduction of the symptoms of RSS in poultry, receiving a challenge with an RSS infectious agent. Preferably, the poultry will display either a therapeutic or protective immunological response such that resistance to new infection will be enhanced and/or the clinical severity of the disease reduced. Such protection may be demonstrated by either a reduction or lack of the symptoms associated with RSS, including, but not limited to, any of those described herein. In some embodiments, a method of the present invention may be used as a vaccination method, vaccinating poultry for the treatment and/or prophylaxis of infection by an RSS infectious agent or a related organism. Any of a wide variety of available assays may be used to determine the effectiveness of the vaccination method of the present invention, including, but not limited to, any of those described herein. For example, clinical scores (including, but not limited to, fecal color, diarrhea, abdominal gut fill, and attitude), histopathology, lesion index (including, but not limited, to size and/or number of cystic enteropathic lesions in the small intestine), percent mortality, or weight gain measurement may be used. Such determinations may be in comparison to non-immunized/RSS challenged, non-immunized/non-RSS challenged, and/or immunized/non-RSS challenged control animals.

The viruses, infected cell lines, cell pellets, supernatants, polynucleotide sequences, vectors, polypeptides, antibodies, compositions, vaccines, and/or host cells described may be administered to poultry to prevent RSS and may be administered to poultry at any of a variety of life stages and/or ages; for example, to a breeder hen. The breeder hen may demonstrate serum antibodies that bind to a polypeptide including amino acid residues described herein, and/or reduced symptoms of RSS. The offspring of the breeder hen may demonstrate such antibodies and/or reduced symptoms of RSS.

The viruses, infected cell lines, cell pellets, supernatants, polynucleotide sequences, vectors, polypeptides, antibodies, compositions, vaccines, and/or host cells described herein may be administered to poultry or other animals, to produce antibodies. Other animals include, but are not limited to, mice, rat, donkey, sheep, rabbit, goat, guinea pig, camel, and horse.

Compositions and vaccines of the present invention may be formulated for delivery by any of a variety of routes known in the veterinary arts, such as for example, mucosal, intranasal, intraocular, or oral administration. Compositions and vaccines of the present invention may be formulated for delivery to the respiratory mucosa and may be administered such that it is immediately or eventually brought into contact with the bird's respiratory mucosal membranes. Compositions and vaccines of the present invention may be formulated for delivery by any of a variety of modes known in the veterinary arts, such as for example, spraying or aerolizing. An immunogenic composition or vaccine of the present invention may be administered by any suitable known method of inoculating birds including, but not limited to, nasally, ophthalmically, by injection, in drinking water, in the feed, by exposure, in ovo, maternally, and the like.

The immunogenic composition or vaccine may be administered by mass administration techniques such as by placing the vaccine in drinking water or by spraying the animals' environment. A composition may be administered by spraying an individual or the flock with a solution, such aerosol delivery may involve the administration of the composition incorporated in small liquid particles. Such spray-type particles may have a droplet size ranging from between about 10 to about 100 microns, more preferably, a droplet size from between about <1 to about 50 microns. For the generation of the small particles, conventional spray-apparatus and aerosol generators may be used, such as the commercially available spray generators for knapsack spray, hatchery spray and atomist spray. Administration through drinking water may be carried out using conventional apparatus. When administered by injection, the immunogenic composition or vaccine may be administered parenterally. Parenteral administration includes, for example, administration by intravenous, subcutaneous,

A composition or vaccine of the present invention may be administered to birds before and/or after hatching. Birds may receive such a composition of vaccine at any of a variety of ages. With delivery after hatching, materials may be delivered, for example, about one week after hatching, about two weeks after hatching, about three weeks after hatching, about four weeks after hatching, about five weeks after hatching, about six weeks after hatching, or any range thereof. For in ovo administration, materials may be delivered about seventeen days of incubation, about eighteen days of incubation, about nineteen days of incubation, about twenty days of incubation, and any range thereof.

In some embodiments, a variety of compositions may be administered. For example, a live formulation of a virus, infected cell line, cell pellet, or cell culture supernatant may be administered first, followed by the administration of a formulation of a killed or inactivated virus, infected cell line, cell pellet, cell culture supernatant or polynucleotide sequence, vector, or polypeptide. Or, for example, a formulation of a killed or inactivated virus, infected cell line, cell pellet, cell culture supernatant or polynucleotide sequence, vector, or polypeptide may be administered followed by the administration of a live formulation of a virus, infected cell line, cell pellet, or cell culture supernatant.

The present invention also includes methods for the detection of RSS agents and antibodies to RSS, including the detection of an RSS infection, detection of previous exposure of an animal to an RSS agent, and/or a determination of the effectiveness of an RSS vaccination effort, or other RSS-control effort, in an animal or a population of animals. The present invention includes methods of detecting or determining exposure of a subject to an RSS infectious agent, the method including detecting the presence of an antibody that binds to a virus, infected cell line, cell pellet, supernatant, polypeptide, and/or host cell as described herein. Antibodies may be detected in samples obtained from the subject, including a biological sample, such as, for example, a tissue or fluid sample isolated from a subject, including but not limited to, for example, blood, plasma, serum, fecal matter, urine, bone marrow, bile, spinal fluid, lymph tissue and lymph fluid, samples of the skin, external secretions of the skin, respiratory, intestinal, and genitourinary tracts, tears, saliva, milk, blood cells, organs, biopsies, or eggs. One or more polypeptides as described herein may be labeled with one or more of the detectable markers known to the skilled artisan. In some aspects, a polypeptide may be bound to a solid substrate. A polypeptide may be included as positive and/or negative controls in antibody based detection methods and kits. In some embodiments, sera from specific pathogen free (SPF) poultry may serve as a negative control.

The present invention includes methods of detecting an RSS infectious agent in a biological or environmental sample by contacting the sample with one or more of the antibodies described herein. As used herein, a biological sample refers to a sample of tissue or fluid isolated from a subject, including but not limited to, for example, blood, plasma, serum, fecal matter, urine, bone marrow, bile, spinal fluid, lymph tissue and lymph fluid, samples of the skin, external secretions of the skin, respiratory, intestinal, and genitourinary tracts, tears, saliva, milk, blood cells, organs, biopsies and also samples of in vitro cell culture constituents including but not limited to conditioned media resulting from the growth of cells and tissues in culture medium, e.g., recombinant cells, and cell components.

Antibodies may be detected by any of a variety of methods, including, but not limited to, the methods described herein and any suitable method available to the skilled artisan. Immunoassays that can be used include, but are not limited to, competitive and non-competitive assay systems using techniques such as BIAcore analysis, FACS (Fluorescence activated cell sorter) analysis, immunofluorescence, immunocytochemistry, Western blots, radio-immunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art. With any of the methods of the present invention, the intensity of a signal from an anti-RSS antibody may be indicative of the relative amount of the anti-RSS antibody in a sample when compared to a positive and negative control reading.

Methods of the present invention may employ detecting the hybridization of a polynucleotide of the present invention to a sample. Such a method may employ producing a polymerase chain reaction (PCR) amplification utilizing at one or more of the oligonucleotide primers described herein. Such a method may employ producing a polymerase chain reaction (PCR) amplification utilizing a primer pair described herein. Such methods may be used for detecting an RSS infectious agent in a biological or environmental sample.

Viruses, polypeptides, polynucleotides, and/or antibodies may be labeled with one or more of the detectable markers known to the skilled artisan. In some aspects, the viruses, polypeptides, polynucleotides, and/or antibodies may be bound to a solid substrate.

Any of the diagnostic methods of the present invention may include the additional step of providing a report or print out of the results. The sample may be any sample in which RSS antibodies, antigens, or nucleotides are present, for example, a blood, serum or tissue sample. Such methods and kits may provide for the detection of exposure of one or more birds to an RSS infectious agent or an RSS vaccine. Such methods and kits may provide for the determination of the effectiveness of an anti-RSS vaccination or immunization effort of other type of RSS control effort including determining if a sera sample from an individual binds to a polypeptide as described herein. Such methods and kits may provide for the detection of infectious RSS agents in environmental samples.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Isolation and Characterization of a Chicken Astrovirus

### An Etiological Agent of Runting Stunting Syndrome in Broiler Chickens

Despite descriptions of runting stunting syndrome (RSS) in broiler chickens dating back over 40 years, the etiology is unknown. With the present invention, a chicken astrovirus (CkAstV) from the gut content of chickens affected with runting stunting syndrome, characterized by retarded growth and the cystic lesions in the small intestine of young chickens, has been isolated in the LMH liver cell line. The in vitro characterization showed that a large number of viral particles remained cell associated. Infection of one-day-old broiler chickens with the isolated virus revealed that the virus initially replicated in the cells of the intestinal villi but later in the cells of the crypt of Lieberkühn. During the first passage in broiler chickens, the virus isolate did not induce growth retardation or the cystic lesions in the small intestine. However, serial chicken-to-chicken passages of the virus resulted in an increased virulence as displayed by decreased weight gain and the presence of cystic lesions in the small intestine. Using filtered material from the 5^{th} chicken passage, the CkAstV isolate caused typical signs for RSS in chickens. The data obtained indicate that the isolated virus has the potential to cause RSS in broiler chickens and can be regarded as an etiological agent of the disease. The analysis of the full length sequences of the isolated CkAstV and the CkAstV obtained after the 5^{th} chicken passage revealed only four exchanges in the amino acid sequence of two viral proteins.

The involvement of astroviruses in enteric diseases in mammals and birds has been described, but little is known for their disease mechanism. In chickens, an enteric disease is runting stunting syndrome (RSS) which causes severe weight gain retardation with unknown etiology. There is a critical need to identify the causal agent of RSS to understand the disease mechanism and to develop a preventive intervention. A new chicken astrovirus was isolated in LMH cells and further analyzed the virus growth as well as the virulence in chickens with regard to RSS outcome. The virus exclusively resides in intestinal epithelial cells only for the first few days after infection. More interestingly, its favorable host cells are crypt epithelial cells following initial replication in villous epithelial cells, thus implying virus preference to immature intestinal cells. Based on the assumption that during cell adaptation the virus may lose its pathogenicity and the possibility of reversion to virulence in its natural host environment, RSS was successfully and repeatedly reproduced by passaging CkAstV in chickens. A role of the virus was demonstrated, without other factors from the chicken gut flora. Comparative genome analysis identifies only four amino acid differences, and whether the amino acid differences account for the pathogenicity will be further analyzed.

Astroviruses are small, round, non-enveloped viruses with a positive-sense, single-stranded RNA genome, and belong to the virus family, Astroviridae. Virus particles, 28-30 nm in diameter with a star-like shape, can be observed by electron microscopy. Astroviruses have been isolated from feces in a wide variety of animals (e.g. including humans, cats, cattle, deer, dogs, ducks, mice, pigs, sheep, mink, turkeys, chickens, bats, cheetahs, guinea fowl, rats and marine mammals) and the identification was mostly associated with gastroenteritis in young individuals (Bosch et al., 2011, “Astroviridae,” pp 953-959 In: Virus taxonomy: classification and nomenclature of viruses: Ninth Report of the International Committee on Taxonomy of Viruses. King et al. (Eds.). San Diego: Elsevier). In addition, extraintestinal diseases such as a fatal hepatitis in ducklings were also associated with astroviruses (Fu et al., 2009, J Gen Virol; 90:1104-1108). Astroviruses isolated from birds belong to the genus Avastrovirus which embraces viruses isolated from poultry including chickens, turkeys, and ducks. Astroviruses, specifically avian nephritis virus (ANV) 1, isolated from chickens were initially grouped in the family Picornaviridae, but after determination of the full length genome sequence designated as a member of the Astroviridae family (Imada et al., 2000, J Virol; 74:8487-8493). Based on sequence data, the existence of an ANV 2 (Pantin-Jackwood et al., 2011, Arch Virol; 156:235-244) and ANV 3 have been reported (de Wit et al., 2011, Avian Pathol; 40:453-461). In addition to the chicken astroviruses, three subtypes of turkey astrovirus have been described. Recently, another astrovirus was described from the intestines of chickens affected with runting and stunting syndrome (RSS) (See Example 3 and Kang et al., 2012 Virus Genes. 44:45-50).

While numerous partial sequences of avian astroviruses are available in gene sequence repositories fewer full length sequences have been determined. The first full length nucleotide sequence of a chicken astrovirus was reported for ANV 1 (Imada et al., 2000, J Virol; 74:8487-8493). Two additional full length genome sequences for chicken astroviruses have since been deposited in Genbank (Example 3 (see also Kang et al., 2012, Virus Genes; 44:45-50); and Fu et al., 2009, J Gen Virol; 90:1104-1108). Also, full length sequences for avian astroviruses from ducks, pigeon and turkeys have been published.

A relatively high diversity within the capsid sequences was found to exist between chicken astroviruses, but even more divergence was observed when compared to ANV 1. Baxendale and Mebatsion (2004, Avian Pathol; 33:364-70) and de Wit et al. (2011, Avian Pathol; 40:453-461) described the isolation of chicken astroviruses in cell culture and embryonated eggs, respectively. One chicken astrovirus caused mild clinical signs (Baxendale and Mebatsion, 2004, Avian Pathol; 33:364-70) while de Wit et al. (2011, Avian Pathol; 40:453-461) described clinical signs and death after infection of SPF chickens. Thus the ability to cause disease varies between the isolates, and more research is needed to identify common markers for virulence.

The concept of the general genome organization of astroviruses (5′-noncoding region, ORF1a/ORF1b, ORF2,3′-noncoding region, poly A tail) holds true, thus far, for all published full length sequences. ORF 1a encodes for a protease containing a protease 3C motif, and ORF 1b encodes for the RNA-dependent RNA polymerase (RdRp) which was identified due to the presence of amino acid sequence motifs typical for nucleic acid polymerases (Carter and Willcocks, 1996, Arch. Virol; Suppl. 12:277-285) ORF 2 is the coding sequence for the capsid protein likely translated from a subgenomic messenger RNA (Lewis et al., 1994, J Virol; 68:77-83; Monroe et al., 1993, J Virol; 67:3611-3614). The proposed replication mechanism involves a frameshift slippery sequence in the overlap region between ORF1a and ORF1b. It is thought that this leads to the synthesis of an ORF1a/1b fusion polyprotein (Jiang et al., 1993, Proc. Natl. Acad. Sci. USA; 90:10539-10543; Lewis et al., 1994, J Virol; 68:77-83). Example 3 (see also Kang et al., 2012, Virus Genes; 44:45-50 proposes a different translation mechanism for a newly described chicken astrovirus where the translation initiation for RdRp possibly occurs at the existing start codon of ORF1a.

This example described the isolation of a chicken astrovirus in cell culture and its full length sequence. The pathogenesis of this isolate was evaluated for its ability to induce clinical signs and microscopic lesions, compared to those described for RSS, in commercial broiler chickens.

### Results

Isolation of an Astrovirus from Gut Material from RSS Affected Chickens

Based on the findings that a new chicken astrovirus (CkAstV) plays a role in the etiology of RSS (Example 2 (see also Kang et al., 2012, Avian Pathology; 41:41-50); Example 3 (see also Kang et al., 2012, Virus Genes; 44:45-50); WO 2010/059899; U.S. patent application Ser. No. 13/107,140; and Sellers et al., 2010 Vaccine, 28:1253-1263), experiments were performed to isolate the astrovirus in several cell lines (MDCK, DF1, QM7, Vero, LMH, Sf9) since the isolation of viruses which replicate mainly in the gut are still not very well established for many disease causing agents. The presence of reoviruses and rotaviruses were expected in the gut of chickens, thus the gut filtrate was incubated with virus specific sera to neutralize both viruses. Among the cell lines tested, only inoculated LMH cells showed CPE beginning at passage three. A CPE became visible after 72 hours and was characterized by small round cells. Forty eight hours later, the CPE was 100%. To characterize the CPE-causing agent, LMH cells were infected with a 1:100 dilution of CkAstV-p5 (5^{th} passage) for indirect immunofluorescence using r-anti-CkAstV serum, ck-anti-reovirus serum, and ck-anti-rotavirus serum along with the appropriate FITC labeled species-specific conjugates. The immunofluorescent was observed only in the infected cells incubated with the r-anti-CkAstV serum ((A) of Figure). The uninfected negative controls as well as infected cells incubated with the ck-anti-reovirus or ck-anti-rotavirus serum did not show specific fluorescence. In order to determine whether the CPE-causing agent was indeed a chicken astrovirus, a virus neutralization experiment was performed in LMH cells using serial dilutions of CkAstV with r-anti-CkAstV serum or with serum obtained from the same rabbit prior to immunization. CPE was observed in all tissue culture flasks up to a dilution of 10⁻⁶ in flasks containing the diluted virus incubated with the pre-immune rabbit serum. In contrast, virus dilutions 10⁻⁴ to 10⁻⁶ incubated with r-anti-CkAstV serum showed no CPE and were also negative in an indirect immunofluorescence assay using the r-anti-CkAstV serum, thus indicating a specific neutralization. In order to support the assumption that indeed only a chicken astrovirus was present in the preparation, several PCRs and RT-PCRs specific for chicken reovirus, chicken rotavirus, infectious bursal disease virus, Newcastle disease virus, avian encephalomyelitis virus, infectious bronchitis virus, chicken adenovirus, reticuloendotheliosis virus, chicken anemia virus, Marek's disease virus, infectious laryngotracheitis virus, and fowlpox virus were performed along with appropriate positive controls for each virus. The investigations were performed at the diagnostic virology laboratory at the Poultry Diagnostic and Research Center (College of Veterinary Medicine, University of Georgia, Athens, Ga., USA) using primer pairs designed to detect a broad range of subtypes within each virus analyzed. In addition, oligonucleotides were used for RT-PCR previously described for the amplification of cDNA for the ANV 1, ANV 2, and chicken astrovirus riboprobes or for PCR for a chicken parvovirus (Example 2 and Kang et al., 2012, Avian Pathology; 41:41-50). Only the primer pair CAstVpr-FP/CAstVpr-RP, specific for the chicken astrovirus, revealed a cDNA fragment of the appropriate size indicating that only the chicken astrovirus was likely present in the cell culture supernatants after virus isolation.

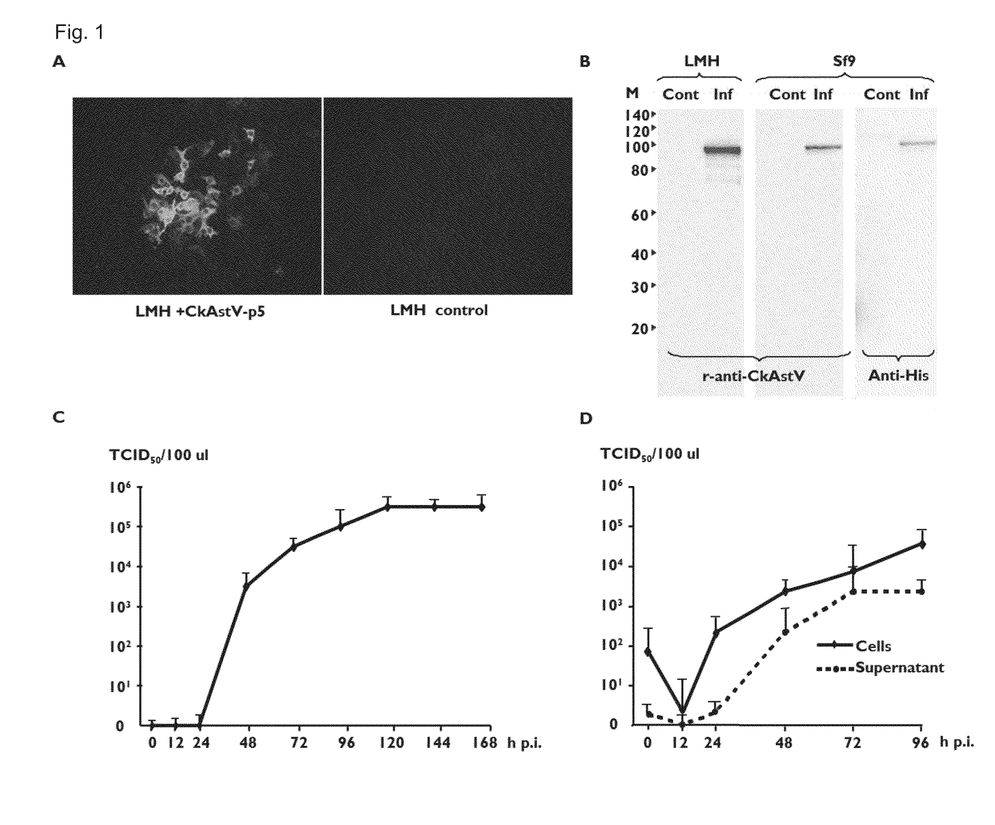

In addition, Western blot analysis was performed for two purposes: 1) to test if the rabbit serum (r-anti-CkAstV) recognized other viral antigens possibly present in CkAstV-p5 infected LMH cells and 2) to test for the recognition of the capsid protein of CkAstV-p5 with the appropriate size of the calculated molecular weight of 98 kDa in infected LMH cells ((B) of FIG. 1). The results showed that the only protein detected by the rabbit serum, r-anti-CkAstV, had an apparent molecular weight of approximately 95-100 kDa which was in the expected range. Since it was the only protein detected in the LMH cells, it is likely that the rabbit serum was specific for immunofluorescent detection of CkAstV-p5 infected LMH cells. In addition, the single protein detected in infected LMH cells was the same size as the recombinant capsid protein from the bacuolovirus infected Sf9 cells. The results from the Western blot and immunofluorescence assay, along with the results of molecular detection methods (RT-PCR, PCR) are strong indicators that r-anti-CkAstV recognized only one virus and that this virus is indeed a chicken astrovirus.

### Characterization of the Chicken Astrovirus in Cell Culture

The titer of CkAstV-p5 was determined to be 10^{5.3}TCID₅₀/100 μl. Based on this titer, growth kinetics were performed using 10² TCID₅₀ per well. The data showed that virus titer increased at 48 hours after infection and reached the highest titer at 120 hours after infection and remained at this level until the end of the study ((C) of FIG. 1). Furthermore, the cell association of the virus was investigated ((D) of FIG. 1). The analysis indicated that one hour after adding the virus to the cell culture (time point 0 h after infection), the virus was primarily associated with the cells, whereas, only a few infectious virus particles were present in the supernatant. This was followed by the viral eclipse at which intracellular and extracellular virus was almost undetectable. The analysis of the following time points revealed a constant increase in viral titers in both cells and supernatants. Based on these results, it appears that one replication cycle was complete between 12 h and 24 h after infection. Furthermore, it became clear that at least 50% of the newly produced viruses remained cell associated and might be of interest when a vaccine is produced.

### Pathogenesis I: Intestinal Replication of the Chicken Astrovirus

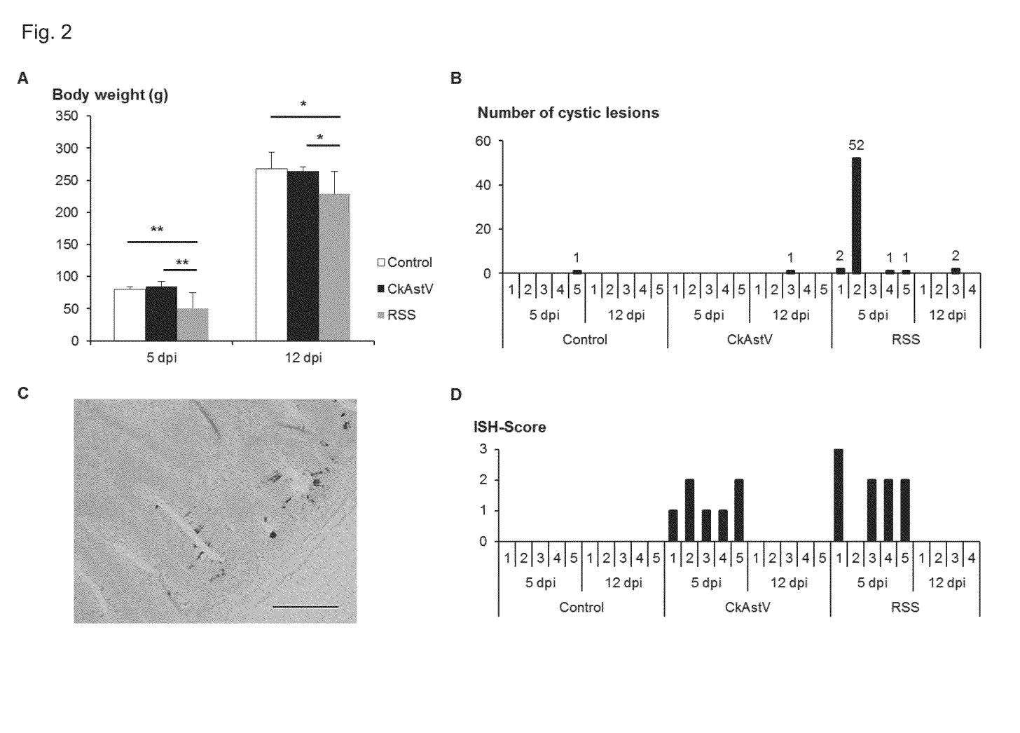

Chickens were infected with CkAstV-p5 (CkAstV), gut content from RSS affected chickens (RSS, positive control), or left untreated (control). Five days and 12 days after infection, body weights, presence of cystic lesions, and the presence of viral RNA in the duodenum were evaluated (FIG. 2). The analysis of the body weights revealed no average body weight difference in chicken with CkAstV-p5 compared to the control chickens, while the chickens inoculated with the gut content from RSS affected chickens showed a significant weight difference at day 5 and 12 after infection ((A) of FIG. 2). Similar results were obtained when the presence of cystic crypt lesions in the duodenum were evaluated ((B) of FIG. 2). Four out of five chickens at day 5 p.i. and one chicken at day 12 p.i. showed cystic lesions after infection with the RSS material. In contrast, one cystic lesion was observed in one chicken from the control group and the CkAstV-p5 infected group at day 5 and 12 p.i., respectively. Therefore, two of the typical RSS signs were not indicated by CkAstV-p5 infection. To examine the virus association in the target tissues, the presence and quantity of chicken astrovirus RNA was investigated by ISH (in situ hybridization) ((C) and (D) of FIG. 2). Interestingly, the RNA was detected in both groups of infected chickens (CkAstV-p5, RSS) at day 5 p.i. with a similar extent; however, the ISH signals were not detected at day 12 p.i. in birds from both groups. No signals were detected in the control birds at any time point. Surprisingly, the majority of RNA positive cells were in the crypt epithelial cells in both RSS and CkAstV inoculated groups. However, a few scattered signals specific for the presence of RNA were also observed along the villi, regardless of the material used for infection (CkAstV-p5, RSS). The estimated ratio of ISH signals between crypt epithelial cells and villi epithelial cells was 95% and 5%, respectively, and indicated that a chicken astrovirus replicated in the crypt in both groups ((C) of FIG. 2). This result was confusing since cystic lesions in a significant number of chickens at day 5 p.i. were only observed in the RSS group but not in the group of chickens infected with the chicken astrovirus alone. Furthermore, intestines with the ISH positive signals contained no or few cystic lesions. Most of all, the bird with the highest cystic lesions (#2 RSS bird) did not show any ISH signals ((B) and (D) of FIG. 2). Thus the link between replication of the investigated CkAstV-p5 and cystic lesions in the duodenum, as observed in RSS affected chickens, was not obvious at this time point and additional experiments needed to be performed.

### Pathogenesis II: Chicken Astrovirus Predominantly in the Crypt Epithelial Cells

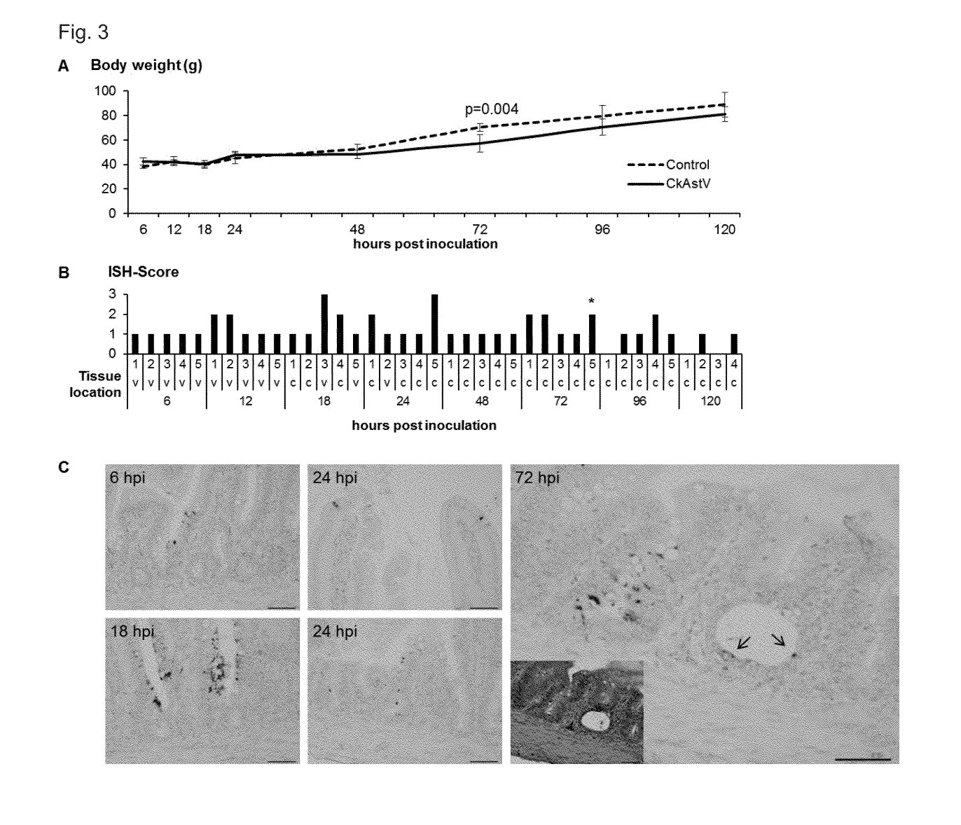

Prompted from the results obtained in the previous experiment (Pathogenesis I), the presence of chicken astrovirus replication was investigated during the first five days after infection (FIG. 3). Mean body weights showed that significant differences were only observed at 72 h p.i. (p<0.05), which was not sufficient to claim a difference in weight gain during the course of the study ((A) of FIG. 3). Cystic crypt lesions were observed in the duodenum of one chicken in the control group in one bird each at 18 h and 24 h p.i. In the group of chickens infected with the CkAstV-p5, one chicken at 72 h p.i. showed two cystic lesions in the duodenal crypt. By ISH, chicken astrovirus nucleic acids were detected only in the CkAstV-p5 infected group. In addition, ANV-1 and ANV-2 nucleic acids were not observed in the tissues by ISH, supporting again that only a chicken astrovirus is present in the inoculated material (Table 1). More interesting was the dynamic of the presence of chicken astrovirus RNA during the course of the experiments ((B) and (C) of FIG. 3). During the first 12 h p.i., viral RNA was exclusively detected in the cells located within the villi. The location of signals changed at 18 h p.i. onwards where initially some ISH signals were still detected in cells located within the villi as well as in the crypt. Within 48 h after infection, viral RNA was predominantly observed in the crypt epithelial cells. Furthermore, the ISH signals were also present in the epithelial cells in the dilated crypt ((C) of FIG. 3). Although the association between the cystic lesions and the presence of virus replication could not be convincingly shown, there is likely a connection between both events and needs to be investigated.

TABLE 1
In situ hybridization of three astroviruses in chickens infected with
chicken astrovirus during the first five days after infection

| | | Hour p.i. | | | | | | | |
|---|---|---|---|---|---|---|---|---|---|
| Group | Probe | 6 | 12 | 18 | 24 | 48 | 72 | 96 | 120 |
| Control | CkAstV | 0/5* | 0/5 | 0/5 | 0/5 | 0/5 | 0/5 | 0/5 | 0/5 |
| | ANV-1 | 0/5 | 0/5 | 0/5 | 0/5 | 0/5 | NT | NT | NT |
| | ANV-2 | 0/5 | 0/5 | 0/5 | 0/5 | 0/5 | NT | NT | NT |
| CkAstV | CkAstV | 5/5 | 5/5 | 5/5 | 5/5 | 5/5 | 5/5 | 4/5 | 2/4 |
| | ANV-1 | 0/5 | 0/5 | 0/5 | 0/5 | 0/5 | NT | NT | NT |
| | ANV-2 | 0/5 | 0/5 | 0/5 | 0/5 | 0/5 | NT | NT | NT |

*Number of positive birds/number of tested birds

### Back Passage I: Serially Passed Virus in Chickens Induced Weight Depression and the Cystic Crypt for RSS

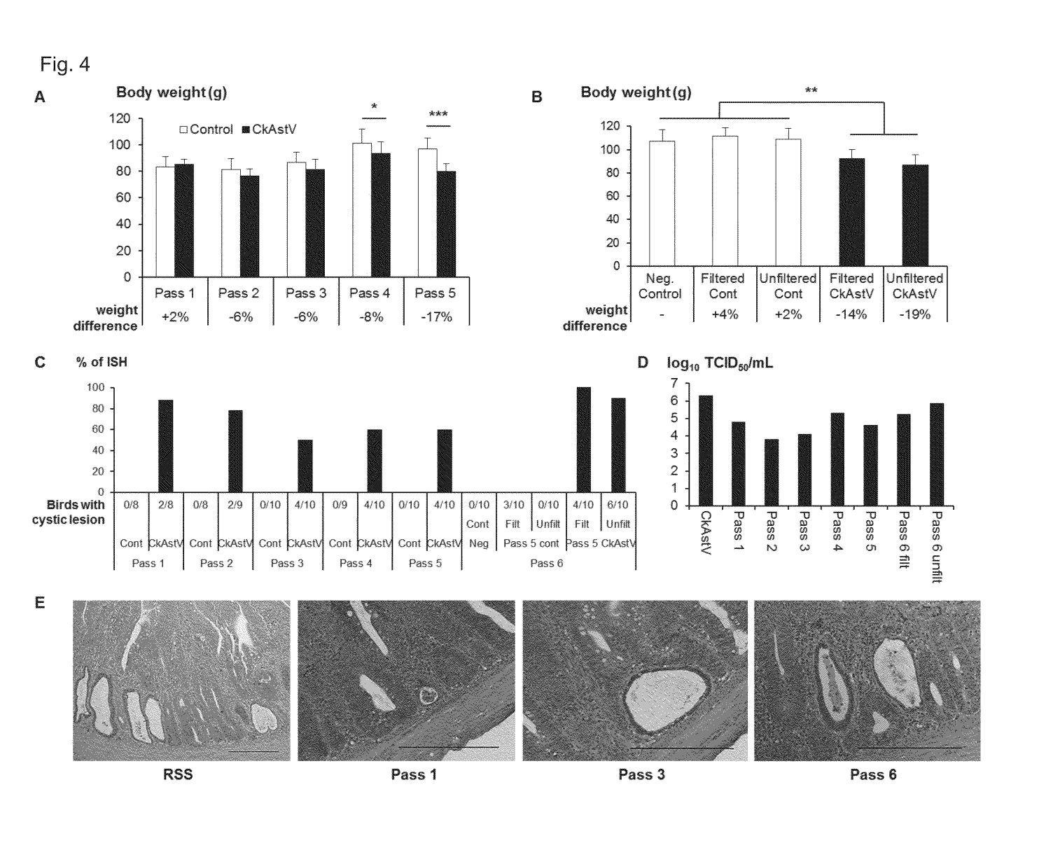

Based on the assumption that the isolated virus was attenuated during cell culture passage, an experiment was performed by passing the chicken astrovirus isolate in broiler chickens using CkAstV-p5 for the first passage and then filtered gut material for the consecutive passages (FIG. 4). The differences in weight gain between control chickens and infected chickens were not significantly different during passage 1, 2, and 3 ((A) of FIG. 4). However, body weights obtained during passage 4 were significantly different (p<0.05) and became even more pronounced during passage 5 with a 17% difference in weight (p<0.001). To investigate the role of bacteria in differences between filtered and unfiltered gut material, a 6^{th} passage including unfiltered material was made from passage 5. Significant differences (p<0.001) were observed in both groups infected with the material containing the chicken astrovirus, regardless whether the material was filtered or not, indicating that likely the chicken astrovirus played the major role for the effect observed ((B) of FIG. 4). The presence of cystic lesions in the duodenum along with the presence of viral RNA as indicated by the presence of ISH signals was evaluated on day 5 ((C) of FIG. 4). Viral RNA was detected in tissues from infected chickens at all passage levels, but not in every infected chicken. This is in agreement with the previous experiment (Pathogenesis II) evaluating the presence of RNA sequentially during the first five days chickens ((B) of FIG. 3). The number of chickens showing lesions also gradually increased during passaging from 2 out of 8 (1^{st} passage) and 2 out of 9 (2^{nd} passage) to 4 out of 10 (3^{rd}, 4^{th}, 5^{th} passage) and 6 out of 10 (6^{th} passage) ((C) of FIG. 3). The presence of lesions observed during the 6^{th} passage in the group that received filtered gut content from the 5^{th} passage of control chickens (3 out of 10) is likely unrelated to the chicken astrovirus since no ISH signal was observed in all three groups representing the controls: uninfected chickens from the negative controls and chickens inoculated with either filtered or unfiltered gut content from the 5^{th} passage control chickens. In addition, the presence or absence of infectious virus was evaluated in the gut of infected chickens by RT-PCR. The RT-PCR for chicken astrovirus RNA was negative for all chickens in the negative control groups, as well as, in the three groups representing the negative controls during the 6^{th} passage (negative control, filtered control, unfiltered control). The gut content from the infected chickens was positive in all groups infected with virus-containing gut content while the negative controls remained negative. From each passage, the gut content was filtered, and the TCID₅₀ was determined ((D) of FIG. 4). Since the determination of the virus titer was performed by indirect immunofluorescence using r-anti-CkAstV, it can be concluded that indeed the chicken astrovirus was quantified. The TCID₅₀ of Ck-AstVp5 used for infection was 10^{6.3}/ml. The titers in the sequential gut samples were between 10^{3.8}/ml (passage 2) to 10^{5.9}/ml (passage 6). The determination can only serve as estimation since there was volume and tissue location variation between the gut samples during necropsy. Cystic lesions in the crypt of the duodenal loop ((E) of FIG. 4) in chickens infected with the chicken astrovirus and in subsequent passages showed a similar pattern as observed after infection with the RSS gut material.

### Back Passage II: Increased Pathogenicity During Passage was Independent of Bacteria

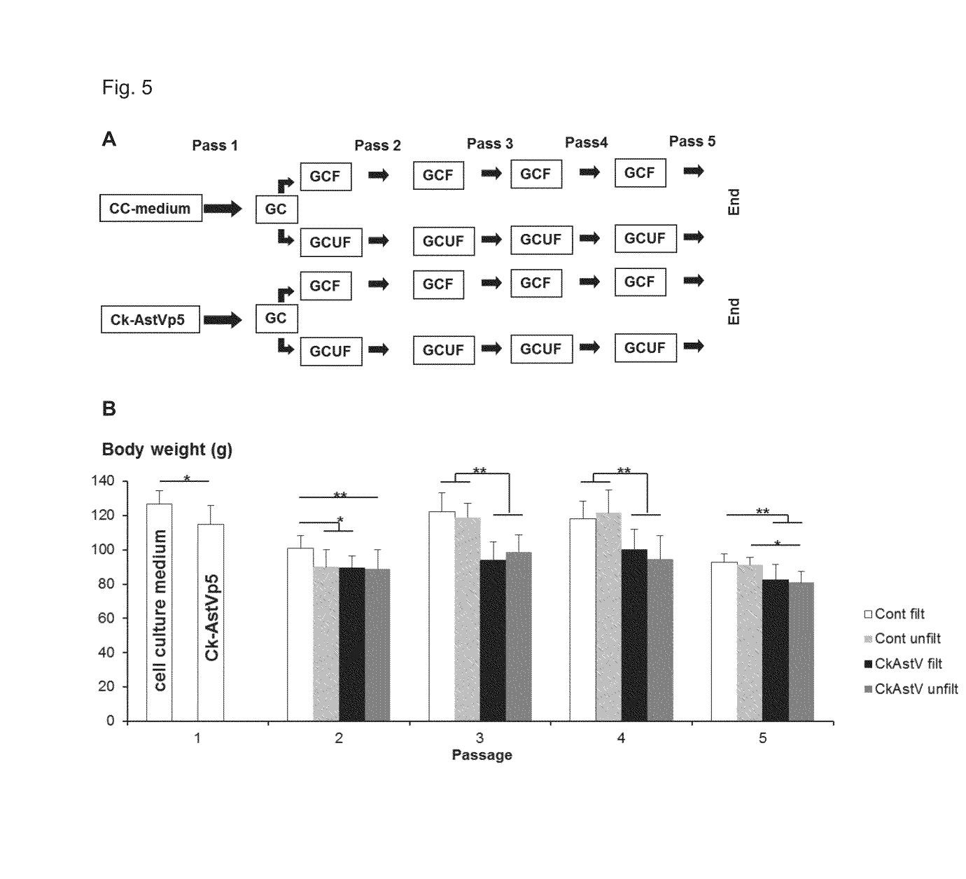

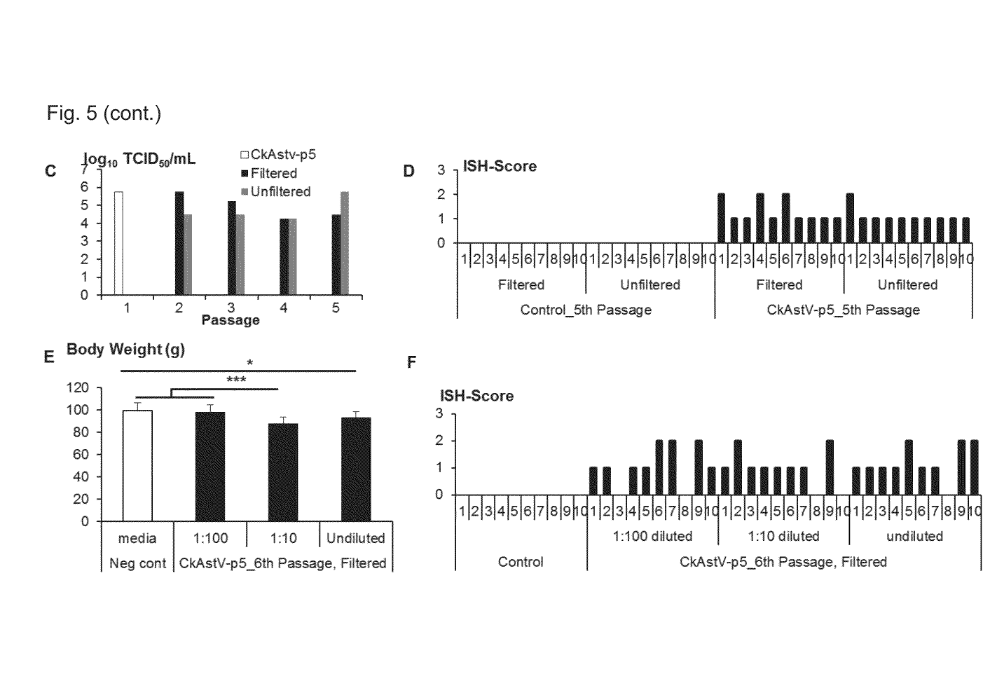

The previous experiment (back passage I) showed that the passage of chicken astrovirus caused reduced weight gain and pathology for RSS. The next experiment was designed to determine whether or not the cause of the RSS induction was related to other factors present in the gut of the broiler chickens since the broiler chickens came from a commercial source. To obtain a deeper insight, the gut contents of control chickens inoculated with cell culture medium and chickens infected with CkAstV-p5 were passaged in parallel as filtered and unfiltered gut contents ((A) of FIG. 5). The weights were measured at day 5 p.i. and the results are summarized in FIG. 5 (see (B)). A significant difference was observed between chickens infected with Ck-AstVp5 and control chickens (p<0.05) as early as in passage 1. During the second passage, a similar picture was observed although chickens belonging to the control group (inoculated with unfiltered gut content) also showed a decreased weight. Environmental monitoring of the isolation units revealed a drop in temperature occurred during the second night of the experiment in the unit of the unfiltered control birds and can be traced back to the weight suppression observed. During the remaining passages (passage 3-5), in the absence of temperature variation, a significant difference in body weight was not observed in the control birds but between control and CkAstV-p5 passaged groups. Likewise no significant differences were observed within groups of the control chickens and groups of CkAstV-p5 infected chickens, regardless of whether the gut content was filtered or not. The viral titers were also determined and ranged between 10^{4.5} TCID₅₀/ml (passage 2, unfiltered group) and 10^{5.75} TCID₅₀/ml (passage 5, unfiltered group), thus a relatively uniform range of virus titers ((C) of FIG. 5). Also the presence of chicken astrovirus RNA, as determined by RT-PCR in every passage and by ISH in the 5th passage ((D) of FIG. 5 and Table 2), was detected only in chicken astrovirus passaged groups. Moreover, 10-fold dilution of the filtered, 5th passaged gut homogenate was sufficient to induce significant body weight retardation in an additional passage experiment (6^{th} passage), and the virus replication was detected by ISH even with 100-fold diluted homogenate inoculation ((E) and (F) of FIG. 5).

TABLE 2
In situ hybridization of three astroviruses in chickens after the
5th passage in chickens

| | | | Probes | | |
|---|---|---|---|---|---|
| Group | | | CkAstV | ANV-1 | ANV-2 |
| Control | Filtrate | P5-1 | 0/10 | 0/10 | 0/10 |
| | Unfilt. | P5-2 | 0/9 | 0/9 | 0/9 |
| CkAstV-p5-Ckp5 | Filtrate | P5-3 | 10/10 | 0/10 | 0/10 |
| | Unfilt. | P5-4 | 10/10 | 0/10 | 0/10 |

*Number of positive birds/number of tested birds

### Analysis of the Nucleotide and Amino Acid Sequences

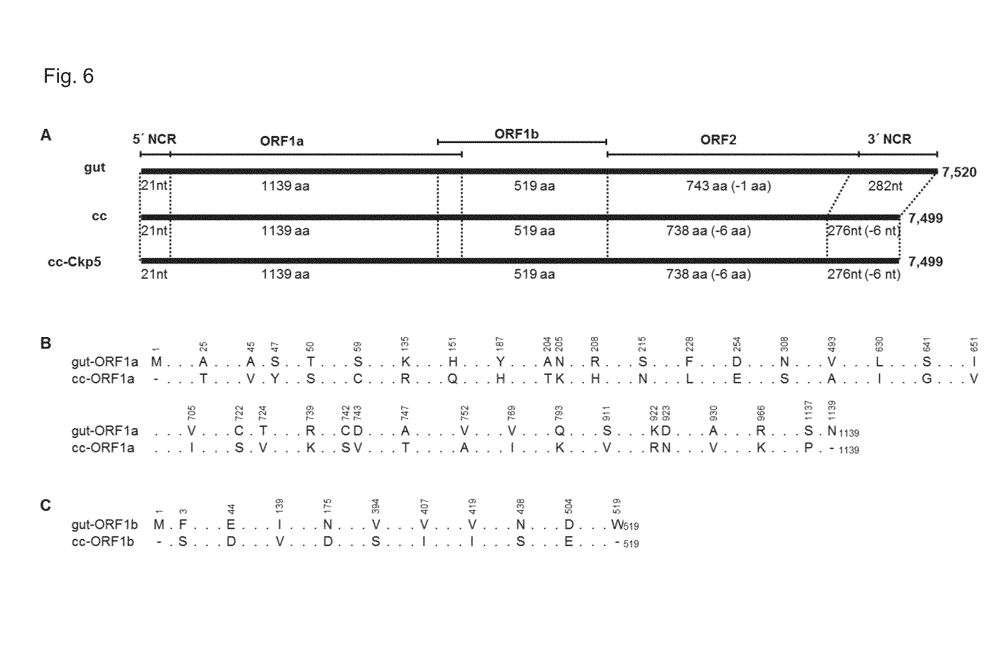

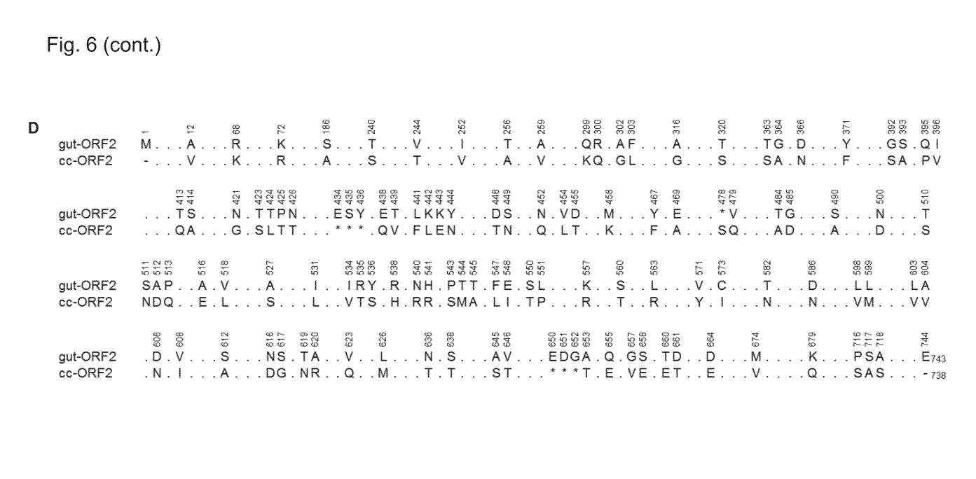

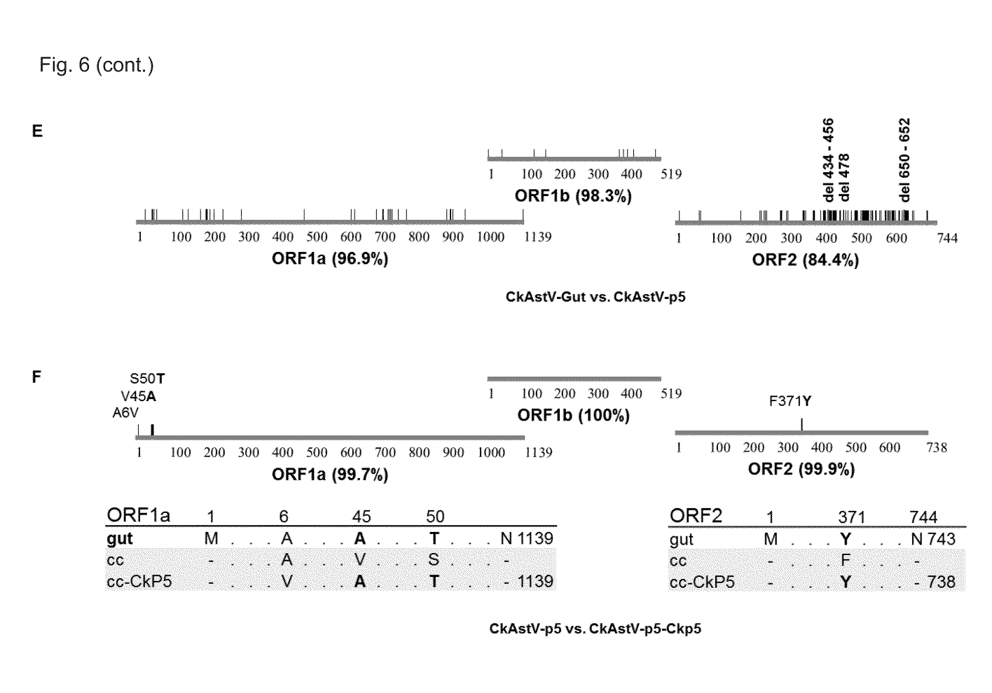

The nucleotide, as well as, amino acid sequences of a new chicken astrovirus present in the gut of RSS-infected chickens (CkAstV-Gut) are described in Examples 3 and 4 (see also, Kang et al., 2012, Virus Genes; 44:45-50). The sequence of this virus served as the basis for the full length sequence determination for the virus isolated in cell culture (CkAstV-p5) and the virus following the fifth passage in chickens (CkAstV-p5-Ckp5) from the back passage experiment. The full length genomic nucleotide sequences of CkAstV-p5 (SEQ ID NO:2) and CkAstV-p5-Ckp5 (SEQ ID NO:3) were determined to be the same length (7,499 nucleotides without poly-A tail sequence), and both were 21 nucleotides shorter than the nucleotide sequence of the original CkAstV-Gut (SEQ ID NO:1) (see (A) of FIG. 6). The 21 nucleotide difference was caused by a deletion of six amino acids (aa434-456, 650-652) within the coding region of the capsid protein (ORF2) and a six nucleotide deletion in the 3′-noncoding region in CkAstV-p5-Ckp5 compared to CkAstV-p5 ((E) of FIG. 6). In addition, within the ORF2 coding region of CkAstV-Gut, one amino acid was deleted (aa478) ((D) and (E) of FIG. 6). The overall homology of the nucleotide sequences between CkAstV-Gut and CkAstV-p5 was 85%. The analysis of the amino acid sequences of the single ORFs (ORF1a encodes for the nonstructural polyprotein, ORF1b encodes for the RNA dependent RNA polymerase, ORF2 encodes for the capsid protein) are shown in FIG. 6 (see (E) and (F). The highest number of exchanges between CkAstV-Gut and CkAstV-p5 was observed in ORF2 (113 aa), followed by the ORF1a (35 aa) and ORF1b (9 aa), which resulted in a homology of 84.8%, 96.9%, and 98.3% given the total length of 743 aa (ORF2), 1139 aa (ORF1a), and 519 aa (ORF1b), respectively ((B)-(E) of FIG. 6). Interestingly, most of amino acid exchanges within the ORF2 encoding for the capsid protein were observed in the C-terminal third of the protein. The exchanges between CkAstV-p5 and CkAstV-p5-Ckp5 were analyzed ((F) of FIG. 6). Only a few nucleotide exchanges occurred during the passage and resulted in an almost identical nucleotide sequence with a homology of 99.8%.

The amino acid sequences for the ORF1a of CkAstV-Gut (SEQ ID NO:4), CkAstV-p5 (SEQ ID NO:5), and CkAstV-p5-Ckp5 (SEQ ID NO:6); the ORF1b of CkAstV-Gut (SEQ ID NO:7), CkAstV-p5 (SEQ ID NO:8), and CkAstV-p5-Ckp5 (SEQ ID NO:9); and the ORF2 of CkAstV-Gut (SEQ ID NO:10), CkAstV-p5 (SEQ ID NO:11), and CkAstV-p5-Ckp5 (SEQ ID NO:12) are given in Example 4.

The comparison of the amino acid sequences revealed three amino acid exchanges in ORF1a (A6V, V45A, S50T) and one amino acid exchange in ORF2 (F371Y) which indicated a stable virus during transmission from chicken-to-chicken. Interestingly, two of the three amino acid exchanges observed in ORF1a (V45A, S50T) and the one amino acid exchange in ORF2 (F371Y) were the amino acids present in the amino acid sequence of the CkAstV-Gut (see (F) of FIG. 6).

### Discussion

The isolation of a chicken virus in an in vitro system, such as embryonated eggs or cell culture, depends on many factors and cannot be predictable. In addition, few diagnostic tools for detection of avian enteric viruses are readily available. As shown in this example, the isolated CkAstV-p5 infected and subsequently replicated only in one cell line evaluated, the liver derived LMH cell. The propagation of CkAstV in LMH cells was previously described (Baxendale and Mebatsion, 2004, Avian Pathol; 33:364-70; Smyth et al., 2012, Avian Pathol; 41:151-159; de Wit et al., 2011, Avian Pathol; 40:453-461) and seemed to be a suitable host cell for the isolation of CkAstV from intestinal contents. In contrast to isolation of astroviruses from bovine, swine, and some human origin samples (Aroonprasert et al., 1989, Vet Microbiol; 19:113-25; Taylor et al., 1997, J Virol Methods; 0.67(1):13-8; Indik et al., 2006, Vet Microbiol; 117:276-283), trypsin was not necessary for the isolation of the CkAstV described here. The isolation of some human astroviruses from clinical samples has been successful in the absence of trypsin (Taylor et al., 1997, J Virol Methods; 67(1):13-8).

Production of a recombinant protein targeting a structural viral protein allowed the generation of a virus specific antiserum in a heterologous host and a means of direct identification of the targeted virus. The availability of the rabbit serum (r-anti-CkAstV), previously generated, was very helpful for the detection of the CkAstV-p5 by immunofluorescence. The serum was generated against a recombinant capsid protein of CkAstV (WO 2010/059899; U.S. patent application Ser. No. 13/107,140; and Sellers et al., 2010, Vaccine; 28:1253-1263) in an SPF rabbit and thus cross reactivity with other chicken pathogens was unlikely. Verification of the specific reactivity was confirmed by Western blot of CkAstV-p5 infected LMH cells and a single band, representing the capsid protein of CkAstV-p5, was observed. In addition, the serum was also able to neutralize 100 TCID₅₀ of the CkAstV-p5 up to a dilution of 2⁻¹³, indicating its specificity for the isolated virus. It would be interesting to determine whether or not the r-anti-CkAstV serum was also able to react with another CkAstV, isolated in LMH cells, (Baxendale and Mebatsion, 2004, Avian Pathol; 33:364-70; Smyth et al., 2012, Avian Pathol; 41:151-159, de Wit et al., 2011, Avian Pathol; 40:453-461) to determine the antigenic relatedness between the different CkAstVs. Replication kinetics of CkAstV-p5 in LMH cells showed that more virus particles remained cell associated compared to virus present in the supernatant. This would be an important factor to consider for virus production in the event that it would be used for vaccine production.

Although different viruses associated with RSS have been described (Otto et al., 2006 Avian Diseases, 50:411-418; Banyai et al., 2011, Virus Genes; 42:82-89; Zsak et al., 2008, Avian Pathol; 37:435-441; Palade et al., 2011, Avian Pathol; 40:191-197), the isolation of the appropriate virus and the reproduction of the clinical RSS and cystic lesions in the duodenum has not been shown. Indirect evidence that the CkAstV-p5 might be a primary causative agent was provided by Sellers et al., (WO 2010/059899; U.S. patent application Ser. No. 13/107,140; and Sellers et al., 2010, Vaccine; 28:1253-1263) when the vaccination of broiler breeders with the recombinant capsid protein of CkAstV mitigated the outcome of RSS in the offspring, likely due to the presence of CkAstV-specific antibodies. However, initial infection experiments in chickens (Pathogenesis I) did not show clinical signs and significant cystic lesions in the crypts of Lieberkühn. Nevertheless, the virus replication was detected in the crypt. Since the crypt epithelial cells actively proliferate and migrate toward the villi, the dynamics of virus replication in the gut was investigated (Pathogenesis II). This finding indicates that initially cells of the villi were susceptible but later became refractory to infection with CkAstV-p5, while the virus was able to spread to and replicate in the crypt. Nonetheless, the absence of cystic lesions and the presence of virus replication in the crypt of the duodenum were still puzzling. It appeared as though the virus was attenuated during virus isolation in cell culture, and the serial passage in chickens may reverse the attenuated phenotype. Indeed, back passage experiments I and II showed that initial passages caused neither significant numbers of cystic lesions nor significant differences in weight between infected broiler chickens and the controls. However, following five passages, both cystic lesions and a significant difference in weight was observed. Passage of both bacterial/viral and only viral material for a 6^{th} passage proved the difference was indeed caused by the viral load. In both cases, the phenotype was reproduced, thus the role of bacterial microflora in RSS was not necessary. Furthermore, data obtained from the serial passaged unfiltered gut content (back passage II) from both negative controls and CkAst-p5 infected chickens supported the finding that indeed the isolated CkAstV-p5 might be at least one causative agent for RSS by excluding other factors from the gut. Since the CkAstV-p5 isolate was tested by both molecular (PCR, RT-PCR) as well as immunological (FA, ELISA) tests for a number of chicken pathogens and found to be negative, it is likely that the virus described here is a pathogen capable of inducing RSS. Moreover, other astroviruses known in chickens such as ANV-1 and ANV-2 were also negative in the virus infected chickens (Tables 1 and 2), thus providing another support for the dependent role of CkAstV-p5 in RSS.

Interestingly, when the nucleotide and amino acid sequences of CkAstV-p5 were compared to the nucleotide and amino acid sequences of CkAstV (Example 3 and Kang et al., 2012 Virus Genes; 44:45-50), analysis revealed that the two viruses were not the same virus even though the source for both viruses was the same. This may indicate that at least two different CkAstVs were present in the original sample. However, the r-anti-CkAstV serum neutralized the CkAstV-p5 isolate even though the amino acid sequence of the recombinant protein was based on the sequence for the chicken astrovirus from the gut. This indicated that the neutralizing antibody-inducing epitopes are either located primarily in the N-terminal region of the capsid protein, where the highest homology was observed, or that the neutralizing antibody-inducing epitopes were still present in the variable region (Krishna, 2005, Viral Immunol; 18(1):17-26) even though a divergence between the capsid proteins of both viruses was identified. The findings of this example are very interesting. The cell culture adapted CkAstV-p5 contained several deletions in the capsid encoding sequence compared to the sequence of the CkAstV from the gut. Also, the 3′-noncoding region of CkAstV-p5 was six nucleotides shorter. Whether these deletions influence the replication of CkAstV-p5 in cell culture needs to be elucidated by reverse genetics. A similar phenotype was described for a human astrovirus where a 45 nucleotide deletion within the viral genome was also observed after adaptation to cell culture (Willcocks et al., 1994, J Virol; 68:6057-6058). In addition, during the back passage of CkAstV-p5 in broiler chickens, amino acid sequences reverted from the sequence observed in the CkAstV-p5 to those observed in the astrovirus sequences obtained from the gut samples (ORF 1a: V45A, S50T; ORF2: F371Y). Since these were almost the only aa exchanges observed during back passage of Ck-AstV-p5, it will be interesting to determine whether or not amino acid exchanges influence the disease outcome, since clinical signs of RSS were reproduced during the back passage of the virus. To this end, a reverse genetics system will be established for CkAstV, as previously described (Imada et al., 2000, J Virol; 74:8487-8493) for ANV 1, to analyze the importance of the differences observed between the each virus and improve our biological understanding of CkAstV.

### Material and Methods

### Cells

The following cell lines were used for isolation of a chicken astrovirus: Madin Darby canine kidney cells (MDCK, CRL-2285, ATCC, Manassas, Va.), DF1, a chicken fibroblastoid cell line (CRL-12203, ATCC), Vero cells (CRL-1587; ATCC), and LMH, a chicken hepatocellular carcinoma epithelial cell line (CRL-2117, ATCC). The cells listed above were grown in Dulbecco's modified Eagles' medium with 4.5 g/liter glucose (DMEM-4.5; Thermo Scientific, Waltham, Mass.) supplemented with 10% fetal bovine serum (FBS; Mediatech, Manassas, Va.). A quail muscle cell line (QM-7; RIE 466; Collection of Cell Lines in Veterinary Medicine [CCLV], Insel Riems, Germany) was also used and propagated in a mixture of equal parts of minimal essential medium (MEM; Invitrogen, Carlsbad, Calif.) with Earle's balanced salt solution and MEM with Hanks' balanced salt solution (Invitrogen, Carlsbad, Calif.), supplemented with 10% FBS. All cells excluding the LMH cells were cultivated in a humidified incubator at 37° C. with 5% CO₂. LMH cells were cultivated in a humidified incubator at 39° C. with 5% CO₂. The insect cell line of Spodoptera frugiperda (Sf9; Invitrogen, Carlsbad, Calif.) was cultivated in serum-free SFX-Insect medium (Thermo Scientific, Waltham, Mass.) at 28° C.

### Generation of a Rabbit Antiserum Specific for the Capsid Protein of a New Chicken Astrovirus

To generate a rabbit antiserum, the recombinant capsid protein of a chicken astrovirus, expressed in a baculovirus system, was purified (WO 2010/059899; U.S. patent application Ser. No. 13/107,140; and Sellers et al., 2010, Vaccine; 28:1253-1263) and used for immunization of a rabbit at the Polyclonal Antibody Production Service facility (University of Georgia, Athens, Ga.). The resulting rabbit serum was named r-anti-CkAstV serum.

To test the reactivity of r-anti-CkAstV serum Western blot analysis was performed. LMH cells grown in T25 tissue culture flasks were infected with the isolated chicken astrovirus, CkAstV-p5 (see below), at a multiplicity of infection (MOI) of 1. Three days after infection, the cells were trypsinized, resuspended in serum-containing DMEM-4.5 and sedimented at 700×g for 5 min. In addition, Sf 9 cells were infected with the recombinant baculovirus encoding for the capsid protein of a chicken astrovirus (WO 2010/059899; U.S. patent application Ser. No. 13/107,140; Sellers et al., 2010, Vaccine; 28:1253-1263). Five days after infection, the cells were scraped into the medium and pelleted at 700×g for 5 min. The cells were washed once in phosphate buffered saline (PBS) for all cell pellets obtained (Sf9 and LMH cells). After a repeated centrifugation step, the cell pellet was resuspended first in 300 μl PBS and then 300 μl of 2× Laemmli buffer (4% sodium dodecylsulphate (SDS), 20% glycerol, 10% 2-mercaptoethanol, 0.004% bromphenol blue, 0.125 Tris HCL). The lysate was heated at 95° C. for 2 min, centrifuged for 5 min at 13,000×g and the supernatants were transferred into a 1.5 ml reaction tube. The rest of the steps were conducted as described by Sellers et al. (2010, Vaccine; 28:1253-1263). Briefly, the transferred membrane was incubated with r-anti-CkAstV serum or an HRP-conjugated anti-6×His-tag monoclonal antibody (Genscript, Piscataway, N.J., USA). For the detection of r-anti-CkAstV antibody binding, goat anti-rabbit HRP-conjugated antibodies (Sigma-Aldrich, St Louis, Mo., USA) were used.

Virus Isolation from Gut Material of Chickens Affected with RSS

Starting materials were obtained from chickens demonstrating significant clinical signs of RSS, following procedures described in more detail in WO 2010/059899; U.S. patent application Ser. No. 13/107,140; and Sellers et al., 2010, Vaccine; 28:1253-1263). The filtered material was incubated with chicken reovirus (ck-reovirus) and chicken rotavirus (ck-rotavirus) antisera from chickens (Charles River SPAFAS, Wilmington, Mass., USA) in a 1:1:1 ratio for 60 min at 37° C. to neutralize the respective viruses. To determine if the sera used to neutralize the chicken reoviruses and rotaviruses contained antibodies capable of neutralizing the new astrovirus, an ELISA was performed using the recombinant capsid protein of the chicken astrovirus as antigen. The investigated serum samples had OD values below the threshold of 0.2 and were regarded as negative for antibodies to the recombinant chicken astrovirus antigen.

One milliliter of the final material was used for passage in cell cultures (MDCK, DF1, QM7, Vero, LMH, Sf9) propagated in T25 cell culture flasks grown to 80% confluency and incubated for five days. The inoculated cells were examined daily for the presence of a cytopathic effect (CPE) as compared to appropriate negative control cells. The cell culture supernatant was collected following centrifugation at 2,000×g for 10 min. One milliliter from each passage of each cell line was used for a subsequent passage up to passage 4. Cell cultures grown in 24-well tissue culture plates were inoculated with a 1:100 dilution of the material obtained from passage 4. At several time points after inoculation (24 h, 48 h, 72 h, 96 h), cells were fixed with ethanol, air dried, incubated with a 1:300 dilution of r-anti-CkAstV serum and a 1:300 dilution of goat anti-rabbit FITC-conjugated antibodies (Jackson Immunoresearch, West Grove, Pa.), and then overlaid with 50 μl of an anti-fading solution containing 1.25% (w/v) 1,4-diazabicyclo[2.2.2]octane (DABCO, Sigma-Aldrich, St Louis, Mo., USA) in PBS. In parallel, the cell cultures were also incubated with ck-rotavirus antiserum or ck-reovirus antiserum followed by incubation with goat anti-chicken FITC-conjugated antibodies (Jackson Immunoresearch, West Grove, Pa.). Uninfected cells were used as negative controls. The immunofluorescence was evaluated using a Carl Zeiss Axiovert 40 CFL inverted microscope. Just for the passages in LMH cells, one 50 μl aliquot was used for a fifth passage in a T175 tissue culture flask containing LMH cells. Five days after infection, the cells were frozen overnight at −80° C., thawed, and then centrifuged at 2,000×g for 10 min. The supernatant was filtered through a 450 nm syringe filter, aliquoted, and stored at −80° C. and served as inoculation material for subsequent experiments (CkAstV-p5).

### Determination of Virus Titers and Growth Kinetics in LMH Cells

One hundred microliter of 10-fold serially diluted virus was added into each of four wells of a 96 well tissue-culture-plate along with 100 μl of the LMH cell suspension. The 96 well tissue-culture plate was incubated for three days at 39° C. Tissue cultures were evaluated by the immunofluorescence for the detection of specific virus as described above. The viral titer (TCID₅₀/100 μl) was calculated by the method of Reed and Muench (Reed and Muensch, 1938, Am J Epidemiol; 27:493-497).

Replication kinetics was evaluated in LMH cells grown in 24-well tissue culture plates and infected with the virus at a multiplicity of infection (MOI) of 1. To this end, the cells were infected with 250 μl of virus containing medium and incubated for 60 min at 39° C. Next, the virus containing supernatant was removed, cells rinsed once with serum-containing medium, and finally overlaid with 1 ml cell culture medium. At several time points after infection, cells were scraped into the supernatant, the suspension removed and used for the determination of the TCID₅₀. In further experiments, the ratio between cell associated virus and virus released into the supernatant was investigated. For this experiment, the infected cells were prepared as described above, except that after the removal of the supernatant, 1 ml of cell culture medium was added and the cell culture plates were frozen and thawed three times. Both the cell culture supernatants and the medium obtained after the freeze/thaw cycles were stored at −80° C. and the TCID₅₀ for each sample was determined.

A virus neutralization experiment was performed in LMH cells. One hundred microliter of ten-fold serial dilutions of the supernatant up to 10⁻⁸ were incubated for 60 min at 37° C. in a virus neutralization experiment with either the r-anti-CkAstV serum or with serum obtained from the same rabbit prior to immunization. Each sample was inoculated into a T25 tissue culture flask containing LMH cells and incubated for five days and followed by two subsequent passages.

### Animal Experiments

Pathogenesis I. To determine the pathogenic potential of the fifth passage of the chicken astrovirus isolated in cell culture (CkAstV-p5), 38 one-day-old chickens were distributed into three experimental groups: CkAstV-p5, RSS, and negative control. The CkAstV-p5 (n=14) group was inoculated orally with 300 μl of CkAstV-p5 at 10^{6.3} TCID₅₀/ml. The RSS (n=9) group was inoculated orally with 300 μl of the RSS gut homogenate (Sellers et al., 2010, Vaccine; 28:1253-1263; Example 2; and Kang et al., 2012, Avian Pathology; 41:41-50) to serve as the RSS positive control, and the chickens in the negative control group (n=15) were left untreated. All chicken experiments described were performed in HEPA-filtered Horsfall-Bauer isolation units with forced air positive pressure. Water and feed was provided ad libitum. Five birds at five days after infection (p.i.) and remaining birds at 12 days p.i. were humanely euthanized. Each bird was weighed, and the duodenal loop was harvested, fixed in neutral buffered formalin and examined for microscopic lesions and by in situ hybridization.

Pathogenesis II. Based on the results obtained from the first animal experiment (pathogenesis I), a short-term study was performed to monitor presence of virus, weight gain retardation, and microscopic lesions in the duodenal loop during earlier time periods. Forty, one-day-old, chickens were inoculated orally with 300 μl of CkAstV-p5. Five chickens were randomly selected at 6, 12, 18, 24, 48, 72, 96, and 120 hours p.i. An equal number of hatch mates were inoculated with 300 μl of cell culture media to serve as controls. At each time point, chickens were evaluated as described above.

Back passage in chickens (back passage I and II). To investigate whether a change in pathotype and/or genotype of CkAstV-p5 would occur during serial passage in broiler chickens, one-day-old commercial broiler chickens were orally inoculated with CkAstV-p5 as described above. On day 5 p.i., the small intestines of each group were harvested and processed with the same method used for RSS material (Example 2 and Kang et al., 2012, Avian Pathology; 41:41-50). Filtered homogenates from CkAstV-p5 chicken passages 1 through 5 were inoculated orally into chickens to make the next passage. Hatch mates at each passage were inoculated with cell culture media to serve as controls. The virus recovered from the fifth chicken passage was identified as CkAstV-p5-Ckp5. For passage 6, chickens were allocated into 5 groups. One group of chickens was inoculated orally with filtered homogenate from passage 5 negative control chickens, the second group of chickens was inoculated with unfiltered homogenate from the same passage 5 negative controls, the third group of chickens was inoculated with filtered homogenate from the CkAstV-p5-Ckp5 group and the fourth group of chickens was inoculated with unfiltered homogenate from the CkAstV-p5-Ckp5 group. The fifth group of chickens was inoculated with cell culture media to serve as negative controls. On day 5 p.i., the same protocols were conducted as described for the initial passage.

To determine if the serial passage of unfiltered gut material from CkAstV-p5 infection would induce differences in weight gain, another set of passage experiment including unfiltered gut material was conducted (passage II). The scheme of the experiments is depicted in figure S1 with detail description. To confirm the pathogenicity of CkAstV-p5-Ckp5, serial 10-fold dilutions from undiluted up to 10⁻² were inoculated in chickens using the filtered passage 5 material (6^{th} passage).

### Detection of Chicken Astrovirus RNA by RT-PCR

For each animal experiment, RT-PCR was performed in one-day-old hatchmates to confirm the absence of CkAstV RNA and in birds from each experiment to investigate presence or absence of the virus. Homogenates from the duodenal loops of each group were incubated at 95° C. for 10 min and subsequently used for RNA extraction with the Qiagen RNeasy plus mini kit (Qiagen, Valencia, Calif.). The SUPERSCRIPT® One-Step RT-PCR with PLATINUM® Taq DNA Polymerase (Invitrogen, Carlsbad, Calif., USA) was used following the manufacturer's instructions. Primers were designed to amplify a 428 nt cDNA of the capsid protein coding region using oligonucleotides ASTCAP-DIAFP (GATAAGGCTGGGCCGCAGAAGAAGAGG; SEQ ID NO:13) and ASTCAP-DIARP (ACAAATTTAACAACACACCGCTG; SEQ ID NO:14) that were delineated from the CkAstV sequence (SEQ ID NO:1) described in a previous study (NCBI Genbank accession number JF414802). The amplified products were separated on a 1.5% agarose gel.

### Evaluation of the Microscopic Lesions and In Situ Hybridization (ISH)

A portion of descending and ascending loops of the duodenum were prepared for microscopic evaluation and ISH as described in Example 2 (see also Kang et al., 2012, Avian Pathology; 41:41-50). The crypt of Lieberkühn in the duodenum was evaluated using hematoxylin and eosin (H&E) stained slides, and the number of cystic lesions per section was counted in the both loops of duodenal sections. The RNA probe for the CkAstV was generated and the ISH was conducted as described in Example 2 (see also Kang et al., 2012, Avian Pathology; 41:41-50). The location of ISH signals and the microscopic lesions were compared for each tissue using consecutively cut tissue sections.

Full Length Sequences of CkAstV-p5 Before and after Passage in Chickens

For the determination of the full length sequence of the chicken astrovirus isolate, either the cell culture supernatant from passage 5 in LMH cells (CkAstV-p5) and filtered gut material obtained after five consecutive passages of Ck-AstVp5 in chickens (CkAstV-p5-Ckp5) were used. Either cell culture supernatant (Ck-AstVp5) or gut material (Ck-AstVp5-Ckp5) was centrifuged, filtered and chloroform treated, as described above, and used for RNA isolation using High-Pure-RNA-Isolation-Kit (Roche, Diagnostics GmbH, Mannheim, Germany).

Based on the sequence obtained for a new chicken astrovirus from gut samples of RSS affected chickens (Example 3 and Kang et al., 2012, Virus Genes; 44:45-50), several pairs of oligonucleotides were delineated to amplify approximately 800 bp cDNA fragments. RT-PCR was performed using the SUPERSCRIPT™ One-Step RT-PCR with PLATINUM® Taq (Invitrogen, Carlsbad, Calif.). The extreme 5′-end of the virus genome was determined using the 5′ RACE System, Version 2.0 (Invitrogen, Carlsbad, Calif., USA) as described in Example 3 (see also Kang et al., 2012, Virus Genes; 44:45-50). The cDNA fragments obtained were separated on a 1% agarose gel, purified using the QIAquick Gel Extraction Kit (Qiagen, Hilden, Germany) and cloned into the pCR2.1 Topo TA plasmid using the Topo TA cloning kit (Invitrogen). Three recombinant plasmids for each cDNA fragment were sequenced using the BigDye Terminator v3.1 Cycle Sequencing kit (Applied Biosystems, Foster City, Calif., USA) in both directions to obtain a six-fold coverage for each nucleotide.

### Multiple Alignment and Sequence Analysis

Sequence data was analyzed using the DNAStar Lasergene 8 software package (DNASTAR Inc, Madison, Wis., USA) for multiple alignments and in silico translation.

### Data Analysis

Mean body weights between groups were compared by SIGAMSTAT® (SigmaStat for Windows, Jandel Scientific, San Rafael, Calif.). Differences between two groups were conducted by t-test, and results between more than two groups were analyzed using one-way analysis of variance (ANOVA) followed by Fisher LSD for all pairwise multiple comparisons.

### Example 2

### Aetiology of Runting and Stunting Syndrome in Chickens

Currently, the aetiology of runting and stunting syndrome (RSS) in chickens is unknown. With this example, the impact of RSS on weight gain and microscopic lesions in immunological organs and the duodenum, was investigated in 1-day-old commercial broilers at 12 days following exposure to RSS-contaminated litter. Furthermore, the presence of the viral nucleic acids of three astroviruses and one parvovirus was analysed by in situ hybridization from days 1 through 5 post exposure. A 70% decrease in weight was observed in the RSS exposed group at the end of the experiments when compared with the unexposed controls. Lesions in the bursa of Fabricius and thymus were present in both groups but were significantly higher at the end of the study in the RSS-exposed group. In contrast, no significant difference in Harderian gland lesions as observed between the groups. Histological lesions in the duodenum were already present 24 h after exposure in the RSS-exposed group only, peaked at day 4 and declined until the end of the study. Results of the in situ hybridization studies clearly indicate replication of three astroviruses (chicken astrovirus, avian nephritis virus (ANV-1 and ANV-2) in the duodenum but not in other organs evaluated. Chicken astrovirus nucleic acids were detected on days 1 and 2 post exposure, while ANV-1 and ANV-2 nucleic acids were observed on several days during the period investigated. Surprisingly, no viral nucleic acid specific for the chicken parvovirus was observed. The results indicate that astroviruses probably play an important role during RSS due to the concurrence of viral RNA detection and lesions in the duodenum.

The role of these viruses in RSS-affected chickens is still poorly understood. Moreover, the viruses may cause RSS in combination with other viruses or unknown factors. The target cell predilection as well as the pathogenicity of these viruses can also differ. To further complicate identification of the disease aetiology, a steady increase in carcass condemnations for septicaemia toxaemia was observed after the onset of the clinical RSS outbreak in late 2004. While this suggests an association of RSS with immune suppression, it is not entirely clear which comes first. However, due to the extremely early onset of signs, it is possible that the agent(s) causing clinical RSS are themselves directly and profoundly immunosuppressive. Due to the complexity of RSS and the possibility of multiple aetiologies, developing treatment and control strategies are hindered. In previous studies, a novel chicken astrovirus was identified in RSS-affected chickens (WO 2010/059899; U.S. patent application Ser. No. 13/107,140; and Sellers et al., 2010, Vaccine; 28:1253-1263). The role of this astrovirus and other small round viruses may hold an important key to identification of the aetiology/aetiologies and improve our understanding of the pathogenesis of RSS.

This example evaluated the development of microscopic changes over time in the small intestine, bursa of Fabricius, thymus, and Harderian gland in commercial broilers challenged with RSS in a previously described challenge model (WO 2010/059899; U.S. patent application Ser. No. 13/107,140; and Sellers et al., 2010, Vaccine; 28:1253-1263). Using riboprobes designed to hybridize to regions of ANV-1, ANV-2, chicken parvovirus and a novel chicken astrovirus, tissues were examined for the presence of replicating virus using an ISH assay.

### Materials and Methods

Chickens. Three hundred 1-day-old commercial broiler chicks obtained from a commercial flock were randomly separated into two experimental groups consisting of 150 birds each. The number of chickens was necessary to obtain a chicken density comparable with commercial production conditions. The birds came from the same company and had the same genetic background as described before (WO 2010/059899; U.S. patent application Ser. No. 13/107,140; Sellers et al., 2010, Vaccine; 28:1253-1263). Each group of chicks was placed into a separate 10 m² isolation house. Water and feed were provided ad libitum. One group was placed on fresh pine shavings, which served as bedding material. For the RSS challenged group, litter material obtained from the same local commercial broiler farm with a history of clinical RSS was used as previously described (WO 2010/059899; U.S. patent application Ser. No. 13/107,140; and Sellers et al., 2010, Vaccine; 28:1253-1263). Starting at 24 h following placement, five birds were collected randomly daily as a representative sample from each group. Birds were numbered (from 1 to 5) and weighed. After euthanizing the birds with carbon dioxide, the duodenal loop, thymus, bursa of Fabricius, and Harderian glands were collected from each bird and placed separately, by individual bird, in 10% neutral buffered formalin. The container was labelled with the bird number, so that the collected tissues could be linked with the bird weight. The same procedure was repeated until 11 days post placement. At 12 days post placement, the experiment was terminated. At this time, all chickens were euthanized and 30 out of the remaining 95 chickens were weighed and samples collected.

Treatment of the tissue samples. A cross-section of the duodenal loop, just above the tip of the pancreas including the ascending and descending sections of the loop, a section of the bursa of Fabricius, the thymus lobes and the Harderian gland, was placed in 10% buffered formalin for 24 h. The fixed tissues were embedded in paraffin blocks and labelled with the group identification number, age, and bird number. The paraffin-embedded blocks were cut consecutively into 4 mm thick sections for subsequent experiments. Sections placed on regular glass slides were stained with haematoxylin and eosin for light microscopic examination. Sections for ISH were placed on Superfrost/Plus microscopic slides (Fisher Scientific, Pittsburgh, Pa., USA).

Evaluation of the microscopic lesions. For the microscopic evaluation, the presence of cystic formation in the crypts of Lieberkuhn (further designated as cystic lesions) in both parts of the duodenal loop was evaluated and the number of cystic lesions per bird was counted. For the primary immune organs (bursa of Fabricius and thymus) and the secondary immune organ (Harderian gland), the evaluation was performed by microscopic assessment of the lymphocytic population. Since it is difficult to evaluate lymphoid tissues in young birds less than 2 weeks of age due to the high dynamic of the development of the tissues, a subjective scoring system was based on comparing lymphocyte populations in the challenged group versus the control group on a daily basis. As the goal of the study was to gauge differences between the two groups, individuals with the largest lymphocyte population within the control group were considered the reference for the scoring system. The subjective microscopic evaluation of the tissues was expressed on a scale of 0 (normal lymphocyte population) to 3 (severely affected lymphocyte population). For a score of 0, there were no differences in the lymphocyte population versus the selected reference tissue. As reference tissue, a sample of a control bird was chosen that showed the highest density of the lymphocyte population, and thus was automatically scored as 0. For a score of 1, there was a subjectively mild difference (25%) in the lymphocyte population versus the selected reference tissue(s). For a score of 2, there was a subjectively moderate difference (50%) in the lymphocyte population versus the selected reference tissue(s). For a score of 3, there was a subjectively severe difference (75%) in the lymphocyte population versus the selected reference tissue.

Generation of riboprobes. Since astroviruses and a parvovirus have been identified as potentially playing a role in the aetiology of RSS, sequences of these viruses were amplified by reverse transcription-polymerase chain reaction (RT-PCR) for astrovirus and PCR for parvovirus. The initial material used for the preparation of plasmids for the transcription of the riboprobes was gut material obtained from chickens exposed to the RSS-contaminated litter that had been taken at day 12. The gut material was homogenized with FastPrep 24 by Bio101 (MP Biochemicals, Solon, Ohio, USA). The resulting homogenate was centrifuged at 13,000 g at 48° C. for 20 min. The supernatant was taken for either RNA purification using the High-Pure-RNA-Isolation-Kit (Roche Applied Science, Indianapolis, Ind., USA) or DNA purification using the QIAamp DNA Blood Mini Kit (QIAGEN, Hilden, Germany). The extracted nucleic acids were used in the experiments described below.

Oligonucleotides (see Table 3) were designed based on sequences available in the NCBI database for ANV-1 (Genbank accession number AB033998), ANV-2 (Genbank accession number AB046864), chicken astrovirus (Genbank accession number JF414802) and chicken parvovirus (Genbank accession number GU214704). The probes for ANV-1 and ANV-2 were located in the coding sequence of the capsid protein. The probe for the chicken parvovirus was located in the viral VP2 sequence, while the probe for the chicken astrovirus was located in the open reading frame 1a region of the virus. Appropriate restriction enzyme cleavage sites were introduced for linearization prior to the DNA-dependent RNA polymerase reaction using either T7 or T3 phage polymerase. After RT-PCR or PCR, the appropriate cDNA fragments were separated on a 1% gel and gel purified using QIAquick Gel Extraction Kit (Qiagen, Germantown, Md., USA). The purified PCR products were incubated with the appropriate restriction enzymes, gel purified again and ligated into the appropriately cleaved pBluescriptII Phagemid vector (Stratagene, La Jolla, Calif., USA). Plasmids containing the expected inserts were selected by restriction enzyme analysis and those plasmids containing an insert were sequenced as described above. A plasmid containing the target sequence was transformed into Top10 F cells (Invitrogen, Carlsbad, Calif., USA) and plasmid DNA was prepared using the GeneJET™ Plasmid Miniprep Kit (Fermentas, Glen Burnie, Md., USA). The resulting purified plasmids were cleaved with the restriction enzyme EcoRI (ANV-1, ANV-2), or XbaI (chicken astrovirus), purified and subsequently transcribed in vitro using phage polymerase T3 (Applied Biosystems/Ambion, Austin, Tex., USA) to generate an antisense RNA of approximately 500 nucleotides in length. For sense probes, cDNA constructs for ANV-1, ANV-2, and the new chicken astrovirus were linearized with SacI followed by in vitro transcription using T7 RNA-polymerase (Takara, Madison, Wis., USA). For the parvovirus-specific sense probes, one recombinant plasmid for each probe was generated due to the incompatibility of the T7 polymerase for the subsequent transcription reaction. The parvovirus-specific cDNA was amplified with the appropriate primer pair (ChPVpr-FP, ChPVpr-RP, see Table 3). The obtained PCR fragment was cleaved either with HindIII/SacII (sense probe) or EcoRI/SacI (antisense probe) and ligated into the appropriately cleaved pBluescriptII Phagemid vector (Stratagene). Additional probes, as described in WO 2010/059899 and U.S. patent application Ser. No. 13/107,140 (which are herein incorporated by reference in their entireties) may also be used.

The plasmids obtained were cleaved with either HindIII (sense probe) or EcoRI (antisense probe), purified and used for the T3 RNApolymerase reaction. For the polymerase reaction, the DIG RNA Labeling Mix (Roche, Basel, Switzerland) was used in accordance with the manufacturer's instructions. Following the polymerase reaction, the plasmid DNA was degraded by adding 10 u RNAse-free DNAse I (Roche) and subsequently incubating for 60 min at 37 C. The reaction was stopped by the addition of 2 ml of 0.2Methylenediamine tetraacetic acid (pH 8.0). The reaction products were purified using SIGMASPIN™ Post-Reaction Clean-Up Columns (Sigma-Aldrich, St Louis, Mo., USA). The presence of the synthesized RNA probe was evaluated by agarose gel electrophoresis. The riboprobe concentration was determined by comparison with a known amount of a DIG-labelled control RNA (Roche) in a dot-blot assay as described by the manufacturer. The dilution factor for each RNA probe was determined to include 35 ng/ml RNA into each hybridization procedure.

TABLE 3
Oligonucleotides used for amplification of the sequences for the RNA probes

| | | SEQ ID | | | Probe |
|---|---|---|---|---|---|
| Name | Sequence | NO | RE site^{a} | Promoter^{b} | orientation |
| ANV1pr-FP | ggGAATTCTTACAACCCAAAACCTGGGC^{c} | 15 | EcoRI | T3 | Antisense |
| ANV1pr-RP | ggGAGCTCGGGAGTATAGGGTCTTCAGAT | 16 | SacI | T7 | Sense |
| | GG | | | | |
| ANV2pr-FP | ggGAATTCGGACCATTGTGGCAGATCGAA | 17 | EcoRI | T3 | Antisense |
| | GC | | | | |
| ANV2pr-RP | ggGAGCTCGGTGCTGAACCAGTACCTGGC | 18 | SacI | T7 | Sense |
| CAstVpr-FP | ggTCTAGATTCTTGTCTAAAGTTATAACA | 19 | XbaI | T3 | Antisense |
| | GGAACAAAGAT | | | | |
| CAstVpr-RP | ggGAGCTCGGGCTTTTGATTGGTAGAATC | 20 | SacI | T7 | Sense |
| | CTTC | | | | |
| ChPVpr-FP | ggCCGCGGGAATTCGGCAACACTAACGGA | 21 | EcoRI | T3 | Antisense |
| | CAACACG | | | | |
| ChPVpr-RP | ggAAGCTTGAGCTCGGAAAAACAAATGTA | 22 | HindIII | T3 | Sense |
| | GTTTCCC | | | | |

^{a}The restriction enzyme used for the linearization of the plasmid. The sequence is bold in the primer sequence; ^{b}Phage promoter used for the transcription of the viral cDNA for the preparation of the digoxygenin-UTP labelled cRNA probe; ^{c}Sequence of the oligonucleotide used for RT-PCR (ANV-1, ANV-2, chicken astrovirus (CAstV) or PCR (chicken parvovirus (ChPV)). Virus-specific sequences are shown in uppercase letter code and a small clamp sequence is shown in lowercase letter code. The restriction enzyme cleavage sites used for linearization are underlined.

In situ hybridization using tissue samples of RSS-infected and control chickens. The unstained tissue slides were first heated at 70° C. for 10 min and deparaffinized in CITROSOLV™ (Fisher Scientific). Slides were then air-dried thoroughly and tissue sections were rehydrated in 5 mM MgCl₂ in phosphate-buffered saline (PBS) for 10 min. Before enzyme digestion, slides were treated in Tris glycine buffer (0.1 M glycine in 0.2 M Tris, pH 7.5) for 10 min at room temperature (RT) and then incubated with proteinase K (35 mg/ml) in proteinase K buffer (10 mM Tris, pH 7.5, 2 mM CaCl₂) for 15 min at 37° C. The enzymatic reaction was stopped in the Tris glycine buffer. Pre-hybridization solution (5 saline-sodium citrate buffer (SSC) containing 0.75 M NaCl, 0.075 M sodium citrate with 50% formamide, 5% blocking reagent (Roche), 0.1% N-lauroylsarcosine and 0.02% sodium dodecyl sulphate (SDS)) was added to sections for 30 min at 42° C. Seventy microlitres of the hybridization solution, which consisted of the pre-hybridization solution containing the riboprobe (35 ng/ml), was applied directly onto the section and covered with a siliconized cover slip (HYBRISLIP™; Grace Bio-Labs, Bend, Oreg., USA). The hybridization was performed overnight at 42° C. in a humid chamber. The next day, coverslips were removed and slides were washed once at 50° C. in 2 SSC (0.3 M NaCl, 0.03 M sodium citrate) with 1% SDS followed by one wash at 50° C. with 1 SSC (0.15 M NaCl, 0.015 M sodium citrate) with 0.1% SDS and at RT with one wash in 1 SSC followed by one wash with 0.1 SSC (0.015 M NaCl, 0.0015 M sodium citrate) for 30 min each. After the washing steps, slides were treated in buffer I (100 mM Tris HCl, 150 mM NaCl, pH 7.5) for 10 min then incubated with a 300-fold diluted sheep anti-digoxigenin alkaline phosphatase-conjugated Fab2′ (Roche) in Buffer I containing 1% foetal bovine serum and incubated for 2 h at 37° C. After three washes with buffer I, the binding of the conjugate was visualized by adding a chromogen mixture (200 ml NBT/BCIP stock solution [Roche] in 10 ml of 0.1 M Tris HCl, pH 9.5, 0.1 M NaCl). The development of the signal was allowed to progress for 45 to 60 min and was stopped by rinsing the slide in distilled water. Slides were lightly counterstained with Gill's haematoxylin and coverslipped with PERMOUNT™ (Fisher Scientific). The slides were evaluated under a light microscope.

Functionality test for the parvovirus riboprobe. Cells of the chicken fibroblast cell line DF-1 grown in Dulbecco's modified Eagles's medium with 4.5 g/l glucose (DMEM-4.5; Thermo Scientific, Waltham, Mass., USA) supplemented with 10% foetal bovine serum (Mediatech, Manassas, Va., USA) were prepared in eight-well-chamber slides (Lab-TekII CC2TM; Nunc, Roskilde, Denmark) 12 h before transfection. Using the TransITmRNA Transfection Kit (Mirus Bio LLC, Madison, Wis., USA), cells were transfected with either DIG-labelled ChPV sense probe, or non-labelled ChPV sense probe, or mock transfected as negative control following the manufacturer's instructions. Four hours after transfection of the cells, the supernatant was removed, cells were washed once with PBS followed by incubation with 10% neutral buffered formalin containing 5% acetic acid for 30 min at RT. After fixation, cells were washed with PBS and stored in 70% ethanol at 48° C. until use. For ISH, cells were incubated with 5 mM MgCl2 in PBS for 10 min and then in Tris glycine buffer (0.1Mglycine in 0.2MTris, pH 7.5) for 10 min. Cells were incubated with proteinase K (3.5 mg/ml) in proteinase K buffer (10 mM Tris, pH 7.5, 2 mM CaC12) for 15 min at 37° C. The enzymatic reaction was stopped by a rinsing step using Tris glycine buffer. The hybridization with the DIG-labelled hPV antisense probe was performed as described above.

### Results

Cystic lesions in the small intestine were present 24 h after exposure. Determination of the body weights indicated a severe challenge of the birds exposed to the RSS contaminated litter. The average of the body weights was significantly different (PB<0.05) starting from day 3 after exposure. Five days following the start of the experiment, the body weight difference between the groups was approximately 50%. The difference in weight culminated at the end of the study where the median body weight of the chickens exposed to the RSS-contaminated litter was only 30% of the weight of the control group. The microscopic evaluation of the cross-section of the duodenal loop revealed the presence of cystic lesions only in chickens that were exposed to RSS-contaminated litter. No differences between control and RSS-exposed birds were observed during the evaluation of the pancreatic tissues. The average lesion numbers were determined per group and by day. One striking finding was that cystic lesions were observed in four out of five birds examined as early as 24 h after placement. Interestingly, the lesions observed at this early time point already showed the structure previously described (see, for example, Nili et al., 2007, Comp Clin Pathol; 16:161-166). The lesions observed were characterized by dilatation of the crypts of Lieberkuhn in addition to atrophy of the intestinal villi ranging from mild to moderate. Furthermore, hyperplasia of the crypt region was observed. In the dilated crypt lesion, the epithelial cells were markedly flattened. The dilated crypt lumen occasionally contained cellular debris that was composed of degenerated cells and eosinophilic cellular debris. Starting with day 10 after exposure, affected crypts first became surrounded and later replaced by connective tissues. In addition, mineralization was occasionally observed within the lesions. The average number of cystic lesions increased until day 4 after placement. Four days following exposure (days 2, 3, 4 and 7), lesions were observed in all five birds examined, while on the remaining days lesions were observed in approximately 80% of the birds. The median number of lesions peaked at day 4 after exposure and declined until the end of the study. It needs to be mentioned that the variability in the numbers of lesions was extreme, as indicated by the minimum and maximum number of lesions per bird. Furthermore, the data were analysed to determine whether there was a correlation between number of lesions in the duodenal loop and the weight of the same bird. The data of each bird for both values are shown side by side for days 4, 5, and 6 post inoculation (p.i.) since the highest lesion scores were observed at these time points. It became obvious that no direct correlation was observed between these parameters, since birds with a comparatively low body weight showed a low number of lesions (day 5 p.i., Bird 4) while in the opposite case a bird with a comparatively high body weight showed a high number of lesions (day 5 p.i., Bird 5).

Primary lymphoid organs were affected during infection. In order to assay whether exposure to RSS-inducing litter caused changes in two of the primary lymphoid organs (bursa of Fabricius, thymus) and one secondary lymphoid organ (Harderian gland), microscopic evaluations were performed. The results obtained for the bursa of Fabricius showed that the scores for the RSS-litter exposed group increased over the course of the experiment. The scores of the bursa of Fabricius were statistically significantly different between the RSS exposed group and the control group from day 9 onwards. The changes observed in the thymus serving as the second primary lymphoid organ showed a similar trend. Thymus sections of chickens exposed to the contaminated litter presented a significantly higher score from day 6 through day 12 after placement, in comparison with the control group which was placed on fresh shavings. One exception was observed on day 8 after placement where no significant difference in the average lesion scores between both groups was observed. In contrast, the evaluation of the Harderian gland as a representative for a secondary lymphoid organ resulted in no detectable differences between both groups in respect to microscopic tissue changes, except for day 6 after placement where significant differences were observed.

Sequence comparison of the probe nucleotide sequences. The length of the amplified sequence from the intestinal content of RSS-infected chickens encoding for parts of the viral capsid protein for the ANV-1 probe was 516 nucleotides (nt) and for ANV-2 was 521 nt. The homology of these nucleotide sequences to each other was 46%. The similarity to the published ANV-1 capsid encoding sequences (AB033998) was 84% while the ANV-2 sequence showed a similarity of 89% to a published ANV-2 sequence (AB046864). Nucleotide sequence similarities to other poultry astroviruses* turkey astrovirus 1 (EU143848), turkey astrovirus 2 (EU143843), duck astrovirus (NC_{—}012437), chicken astrovirus (JF414802)*were below 30%. The sequence used for the probe for the new chicken astrovirus was also obtained from the intestinal sample. The sequence was located in the region of the new chicken astrovirus encoding for the non-structural protein. A NCBI Genbank Blastn search did locate any similar sequences, and a direct comparison with the appropriate nucleotide sequences for ANV-1 (NC_{—}003790), turkey astrovirus 1 (EU143848), turkey astrovirus 2 (EU143843), and the duck astrovirus (NC_{—}012437) showed a similarity of below 20%. The probe for the amplified parvovirus sequence showed an 88% identity to a recently published chicken parvovirus sequence. In situ hybridization revealed astroviruses as agents for RSS. The ISH was performed on all tissues, which included the bursa of Fabricius, thymus, Harderian gland, and the cross-section of the duodenum including the tip of the pancreas. Samples from day 1 through day 5 were analysed. One major problem with the investigations was that viruses of interest were not isolated in cell culture and thus the availability of a positive control for the ISH to show specificity was limited. To address this problem from the very beginning, all probes were designed so that they would have approximately the same length (ANV-1, 516 nt; ANV-2, 521 nt; chicken astrovirus, 450 nt; chicken parvovirus, 511 nt). Due to the sequence composition, the GC content was approximately 45% and very similar in all probes. For the adjustment of the ISH conditions, DF1 cells were transfected with positive-sense transcripts of each virus (ANV-1, ANV-2, chicken astrovirus, and chicken parvovirus) and cross-tested for specificity with each probe. In addition, the antisense-oriented transcripts were also transfected and probed with the sense probes. The adjustment factor for the specificity was the temperature during the washing procedure, probably due to the GC content and the similar length of probes. Interestingly, for all probes the same temperature could be used without losing specificity. Since the study was designed so that each sample could be traced to the appropriate chicken, association of ISH signals to appropriate changes observed microscopically could be evaluated. The bursa of Fabricius, thymus, tip of the pancreas, and Harderian gland showed no ISH signals, neither those samples obtained from the group that was exposed to the RSS-contaminated litter nor the tissue samples obtained from the negative control group. This indicated that neither one of the three astroviruses nor the chicken parvovirus replicated in these organs. A different result was observed when the samples representing the duodenal loop were investigated with the probes specific for chicken astrovirus, ANV-1, and ANV-2. The sense probes for the astroviruses showed no signals that could be appreciated as positive on slides of two chickens which were positive on day 1 after placement. All antisense probes specific for each of the astroviruses showed a positive signal in a number of samples. This indicated a high specificity of the antisense ISH probes with the respective virus. Furthermore, none of the samples investigated from the negative control group showed a positive ISH signal from any of the birds that were taken on days 1 and 3 after placement regardless of whether the sense or antisense probe was used. The general signature of the astrovirus-specific antisense probes was very similar. The majority of positive signals were observed along the villous epithelial layer although occasionally a few rare signals were seen in the lamina propria as irregular particles. The signals in the villous epithelial cells were localized in the cytoplasm, indicating the expected cytoplasmic replication of the virus. Interestingly, no matter whether tissues contained the dilated crypt lesions, the signals for astroviruses were present neither in the crypt epithelial cells nor in the adjacent tissues of the crypt. The signals were clearly distinguishable, which indicated an intense virus replication. No difference in the strength of the signal among the three different astroviruses was observed. The pattern of the staining might be important for the dynamics of the disease.

The signal for ANV-1 was observed in one chicken at day 1, all five chickens at day 3, no chickens at day 4, and four out of five chickens at day 5. The signal specific for the ANV-2 probe was scattered throughout the investigated time points. Two chickens were positive at days 1 and 3 after exposure to the litter, while at days 2, 4, and 5 after exposure one chicken out of the five investigated chickens showed a positive signal. The chronological presence of viral RNA for the new chicken astrovirus was noted. Four out of the five birds evaluated showed a positive signal 24 h after exposure to the RSS-contaminated litter and only one chicken at day 2. None of the investigated sections showed any signal from day 3 through day 5 after exposure. It needs to be mentioned that the sections were cut consecutively from the paraffin-embedded blocks and on an individual basis. The initial concern was that the probes specific for ANV-1 and ANV-2 might cross-react although the nucleotide sequence similarity was only 46% (see above).

However, the data obtained show that although Birds 1 and 2 at day 1 after exposure showed ISH signals for the ANV-2 probe in a high number of cells, hybridization with the ANV-1 probe remained negative. A similar result was observed with Bird 5 on day 4. The ISH with the ANV-2 probe was positive in a high number of cells but negative for the ANV-1 ISH probe. The opposite result was present in Birds 3 and 5 on day 5 where the ISH probe for the ANV-1 was positive in a high number of cells while the ISH for ANV-2 was negative. A very similar result was observed for the ISH probe of the new chicken astrovirus, where no reaction was observed beyond day 2 following exposure while positive signals were observed with both of the other probes (ANV-1 and ANV-2). The comparison of all three ISH signals on a single-bird basis demonstrated that some birds were positive for two viruses at the same time regardless of the strength of the signal. In addition, it was also observed that the presence of cystic lesions was not necessarily related to the presence of viral RNA as was observed for Bird 4 24 h after exposure. The use of both sense and antisense ISH probes specific for parvovirus resulted in no signal in any samples at any time points investigated. Since this result was not expected, experiments were performed to evaluate whether the antisense parvovirus probe was functional. Thus, DF1 cells were left untransfected, or were transfected with either the labelled sense probe to verify the reaction conditions or were transfected with the unlabelled sense probe and probed with the labelled antisense probe. The results showed clearly that the antisense parvovirus probe was able to bind to the sense parvovirus cRNA. This result was an additional strong indicator that no parvovirus was replicating in the duodenal loop of the investigated intestines.

### Discussion

Although the aetiology for RSS remains unknown, early investigations revealed that RSS probably has a viral aetiology. These observations have been supported by experiments using either filtered intestinal content or chloroform-treated, filtered intestinal content. The latter supported the assumption that the viruses causing RSS are nonenveloped. Initially, reovirus was believed to be the major causative agent for RSS since these viruses have been identified in RSS-affected chickens. In addition, intestinal lesions typical for RSS have been reported in specific pathogen free chickens after infection with reovirus of enteric origin. In contrast, neutralization of reovirus from infective homogenates or vaccination of breeder hens against reovirus did not reduce the severity of RSS. Also viruses from a variety of different virus families (Adenoviridae, Parvoviridae, and Togaviridae) have been associated with RSS. However, the exact aetiology of the disease has not been proven to date but more than one agent has been proposed to be involved in this disease syndrome. Despite viral multiplication in experimentally inoculated birds, no clinical signs or growth retardation were observed in specific pathogen free and broiler chickens infected with a reovirus or parvovirus, but abnormal faeces and reduction in weight gains were observed after infection with the field materials and entero-like viruses. A correlation between the presence of cystic lesions in the intestine and the presence of rotavirus has been described (Otto et al., 2006, Avian Dis; 50(3):411-418). Furthermore, it has been described that infection of specific pathogen free Leghorn chickens with a chicken astrovirus resulted in mild diarrhoea and some distension of the small intestine. In order to investigate the aetiology of RSS, the investigations described here focused on three members of the Astroviridae family and a chicken parvovirus. In addition, the possibility of an immuno-compromising component associated with the disease was investigated.

This example clearly shows that the RSS challenge was severe, as indicated by the dramatic difference in weight gain and presence of cystic lesions in the small intestine. The difference in weight at day 12 p.i. in about 70% of the RSS-litter exposed group was greater than described before (50%) in a very similar challenge model (WO 2010/059899; U.S. patent application Ser. No. 13/107,140; and Sellers et al., 2010, Vaccine; 28:1253-1263). In both cases, the number of birds with cystic lesions at day 12 p.i. was very similar. In this study, 22 out of 30 birds showed cystic lesions compared with 20 out of 30 birds in the study previously described (WO 2010/059899; U.S. patent application Ser. No. 13/107,140; and Sellers et al., 2010, Vaccine; 28:1253-1263). This indicated that the presence of either cystic lesions in the duodenum or decreased weight gain cannot be used as a standalone indicator for RSS, but the presence of lesions and a decreased weight gain can be used as a strong indicator for RSS when a certain number of chickens are included in the study. The latter holds true since chickens with a comparable higher weight showed a high number of lesions while birds with a low weight showed a low number of lesions. Another reason might be that the load of the infectious agent(s) was much higher. But since the exact cause for RSS is unknown it cannot be investigated. Interestingly, the average of the cystic lesions were detected in the small intestine in 80% of the RSS group as early as 24 h following exposure, and peaked at day 4 after exposure but declined until the end of the study. The dynamics for the presence of the lesions was described previously by Smart et al. (Smart et al., 1988, Avian Pathology; 17:617-627), where the first cystic intestinal lesions were observed as early as 3 days after inoculation due to the sampling schedule used in that study. It is possible that lesions in this study were present at an earlier time point. The immediate presence of cystic lesions in the intestine followed by lymphocytic depletion in the immune organs 5 to 6 days later is probably not a direct result of any of the four viruses targeted in this study. The absence of ISH signals in the immune organ supports this notion. These observations indicate rather that RSS-contaminated litter from poultry houses harbors other pathogens that cause lesions in the bursa of Fabricius and the thymus of the chicken. This was not surprising and has been shown before (Reece et al., 1984, Veterinary Record; 115:483-485; Montgomery et al., 1997, Avian Diseases; 41:80-92; and Nili et al., 2007, Comp Clin Pathol; 16:161-166). Another possibility is that an organ is involved which regulates the growth of chickens, such as the pituitary gland that synthesizes the growth hormone. But to test this hypothesis, another set of experiments needs to be performed. Also other organs could be included, such as the kidney and liver, since these organs are important for growth and therefore have the potential for involvement in the disease complex.

The absence of ISH signals using both parvovirus probes provided critical evidence that parvovirus, although described to be present in RSS cases, may not play an important role in the aetiology of the disease. Although Kisary et al. (Kisary et al., 1984, Avian Pathology; 13:339-343) described the presence of parvovirus in guts of chickens that suffered from RSS and showed later that the obtained parvovirus ABU isolate was able to induce growth retardation in chickens, other experiments in broiler chickens using the same ABU isolate could not confirm the results (Decaesstecker et al., 1986, Avian Pathology; 15:769-782) since neither weight depression nor lesions in the intestines were observed. Additional evidence has been reported that implies chicken parvoviruses might play an important role in enteric diseases in poultry since parvoviruses were detected in most cases with an enteric disease in chickens and turkeys. Due to the lack of a virus isolate and induction of the disease complex with such an isolate, however, the evidence presented to date is only circumstantial. As described here, the presence of signals for three members of the family Astroviridae (a new chicken astrovirus, ANV-1, ANV-2) may provide additional in vivo support that members of this virus family play an important role for the induction of the disease. Some known avian astroviruses have previously been detected by RT-PCR in materials obtained from RSS-affected chickens.

This example showed, for the first time, a physical presence of astroviruses at locations in the intestine where RSS-associated lesions were also observed. Initially, we were concerned that ISH probes for ANV-1 and ANV-2 might cross-react with the viral RNA of the other ANV, but the results showed that while ISH signals in the consecutive intestinal sections were present for ANV-1, the next section was negative for both chicken astrovirus and ANV-2, respectively. Although cystic lesions were present soon after exposure to the RSS-contaminated chicken litter, the ISH signals observed were limited to the epithelial cells in the intestine. The results of this study implied that the lesions in the intestine were caused by another viral agent or that cystic enteropathy is coincidentally present but not associated with the outcome of the disease. This hypothesis is supported by the early observation of cystic enteropathy at 24 h after exposure but a lack of ISH staining in the crypts. On the other hand, experimental evidence using a recombinant astrovirus capsid-based vaccine indicated that the new chicken astrovirus might play a role in this disease (WO 2010/059899; U.S. patent application Ser. No. 13/107,140; and Sellers et al., 2010, Vaccine; 28:1253-1263.

Example 2 has also published as Kang et al., “Investigation into the aetiology of runting and stunting syndrome in chickens” Avian Pathol. 2012; 41(1):41-50. doi: 10.1080/03079457.2011.632402, which is hereby incorporated by reference in its entirety.

### Example 3

### Determination of the Full Length Sequence of a Chicken Astrovirus

With this example the genomic RNA of a novel chicken astrovirus was determined. The full length sequence is 7520 nucleotides (SEQ ID NO:1) and encodes three open reading frames (1a, 1b, 2) for three proteins (SEQ ID NO:4, SEQ ID NO:7, and SEQ ID NO:10, respectively). The genomic organization was similar to other astroviruses with two exceptions. The open reading frame of the RNA-dependent RNA polymerase contains its own start codon which is different from other astroviruses described to date, providing evidence for a replication mechanism different than what has previously been described for astroviruses. Furthermore, the stem-loop structure located at the potential ribosomal frameshift signal described for other astroviruses has been shown to be a hairpin structure for the novel chicken astrovirus. Phylogenic analysis of the full length sequence revealed that this chicken astrovirus formed a branch independent from other astroviruses, indicating that this astrovirus is significantly different from astroviruses described to date.

Viruses belonging to the family Astroviridae have a non-enveloped capsid which contains a positive sense, ssRNA genome (E. Mendez, C. F. Arias, in Fields Virology, ed. By D. M. Knipe, P. M. Howley (Lippincott Williams & Wilkins, Philadelphia, 2007), pp. 981-1000). The viruses belong to a large group of small viruses with a diameter of approximately 28-30 nm. The genome length varies between 6.8 and 7.9 kb irrespective of the species of isolation. The genome encodes for three proteins, the nonstructural polyprotein (NS polyprotein), the RNA dependent RNA polymerase (RdRp), and the capsid protein (Jiang et al., 1993, Proc Natl Acad Sci USA; 90:10539-10543). The NS polyprotein and the capsid protein are each encoded by an individual open reading frame (ORF), ORF1a and ORF2, while the RdRP (ORF1b) has been reported to be expressed via a ribosome shift mechanism (Jiang et al., 1993, Proc Natl Acad Sci USA; 90:10539-10543) as a fusion protein to the NS protein (Marczinke et al., 1994, J Virol; 68:5588-5595).

Astroviruses have been isolated worldwide from several mammals (humans, cats, pigs, sheep, bat) as well as birds (ducks, chickens, turkeys) and are associated in general with gastroenteric diseases. While in mammals astroviruses mainly cause diarrhea, in birds astroviruses are associated with wider spectrum of diseases, including diarrhea, hepatitis, and nephritis. One of the diseases in poultry associated with astrovirus is the runting and stunting syndrome (RSS) in chickens. RSS is a transmissible disease of uncertain etiology. RSS affects chickens early in life and is characterized by growth retardation, ruffled feathers, and diarrhea resulting in considerable economic losses especially in commercial broiler production. The syndrome is also known as malabsorption syndrome, infectious stunting syndrome, broiler runting syndrome, and helicopter syndrome (Rebel et al., 2006, World Poultry Sci J; 62:17-30).

Clinical and pathological signs of RSS have been experimentally reproduced using oral inoculation of filtered and non-filtered intestinal homogenates from RSS affected chickens (Sellers et al., 2010, Vaccine; 28:1253-1263; Montgomery et al., 1997, Avian Dis; 41:80-92; Songserm et al., 2000, Avian Dis; 44:556-567; and Songserm et al., 2002, Avian Dis; 46:87-94). Based on preliminary sequence data of the capsid protein, obtained from gut samples collected from chickens experimentally exposed to RSS-contaminated litter, the full length sequence of astrovirus was determined. Comparisons to published astrovirus sequences indicated that the virus which harbors this genome belongs to a chicken astrovirus not previously described.

### Materials and Methods

Generation of material for sequence determination. One-day-old commercial broiler chickens were exposed to chicken litter transported from a commercial farm with chickens exhibiting RSS to a research isolation house, as previously described (WO 2010/059899; U.S. patent application Ser. No. 13/107,140; and Sellers et al., 2010, Vaccine; 28:1253-1263). Chickens from this study were euthanatized with CO₂ and the small intestine was harvested and homogenized with sterile phosphate buffered saline (PBS) at a 1:3 ratio (w/v) in a blender. The resulting homogenate was centrifuged at 3500×9 g for 20 min at 4° C. The supernatant obtained was centrifuged a second time at 160009 g for 20 min at 4° C., followed by a sequential filtration through a 0.45 μm and subsequently through a 0.22 μm filter (Whatman, Florham Park, N.J., USA). The filtrate was treated with chloroform and used for RNA purification using the High Pure RNA Isolation-Kit (Roche, Diagnostics GmbH, Mannheim, Germany). RNA was stored at −80° C. until use.

Determination of the sequence of a novel chicken astrovirus from gut samples. RNA isolated and purified from the gut homogenate described above was used for 5′-rapid amplification of cDNA using the 50 RACE System, Version 2.0 (Invitrogen, Carlsbad, Calif., USA). The first primer used for the initial cDNA synthesis was located inside the open reading frame (ORF) of the capsid protein from a previously reported chicken astrovirus (WO 2010/059899; U.S. patent application Ser. No. 13/107,140; and Sellers et al., 2010, Vaccine; 28:1253-1263). The subsequent PCR was performed with a nested astrovirus-specific primer and the anchor primer from the 50 RACE System. The RT-PCR fragment obtained was gel eluted and purified using the QIAquick Gel Extraction Kit (Qiagen Sciences, Md, USA) and cloned into the pCR2.1 plasmid using the TOPO TA cloning kit (Invitrogen) and transformed into competent E. coli. The recombinant plasmids obtained were sequenced using the BigDye Terminator v3.1 Cycle Sequencing kit (Applied Biosystems, Foster City, Calif., USA). Based on the novel sequence obtained, two novel astrovirus-specific oligonucleotides were delineated and used for the next 5′-RACE amplification. One was used for the initial cDNA synthesis, while the second oligonucleotide was used as nested primer for the subsequent PCR. Using the primer walking approach, the full length sequence was determined. To determine the extreme 5′ end of the viral genome, different 50 RACE reactions were performed as described by Mundt and Muller (Mundt and Muller, 1995, Virology; 209:10-18). In brief, for the determination of the first 50-end nucleotide, the deoxynucleotide tailing reaction was performed using either dCTP or dGTP. The subsequent PCR was appropriately performed with either the anchor primer (dCTP-tailing) or a poly-C primer (dGTP tailing). Since the primer walking procedure was performed on a non-defined mixture present in the gut, the full length sequence was confirmed by amplification of overlapping 1 kb fragments using oligonucleotides delineated from the previously determined sequence. The RT-PCR fragments obtained were cloned and at least 3 plasmids were sequenced in both directions, obtaining a six fold coverage of the sequence.

Multiple alignment and sequence analysis Sequence data was analyzed using the DNAStar Lasergene 8 software package (DNASTAR Inc, Madison, Wis., USA) for sequence alignments and in silico translation to amino acid sequences. Phylogenic analysis was performed using the MEGA-4.1 software (Tamura et al., 2007, Mol Biol Evol; 24:1596-1599), available as freeware online (megasoftware.net/mega4/mega41.html). The RNA secondary structure was determined using the RNA secondary structure prediction software available online at genebee.msu.su/services/rna2_reduced.html.

### Results and Discussion

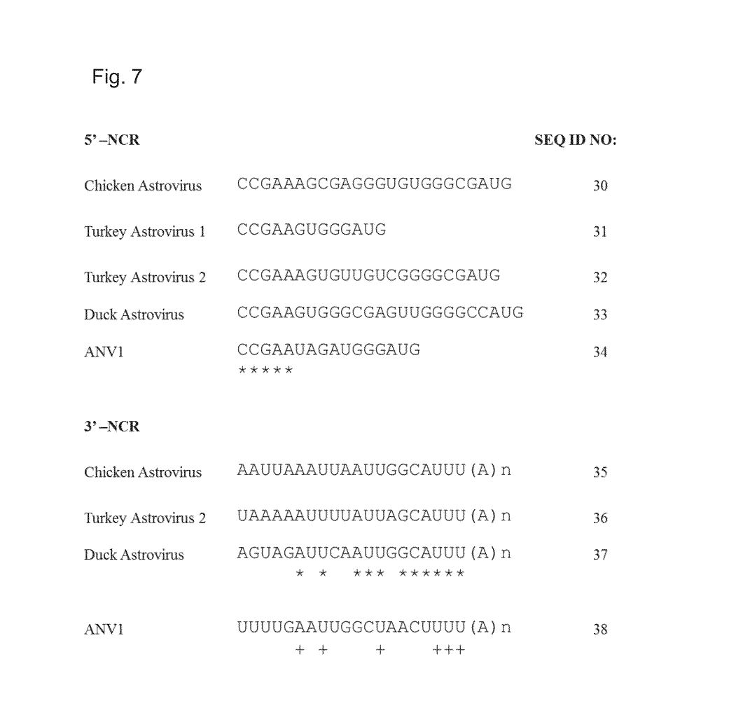

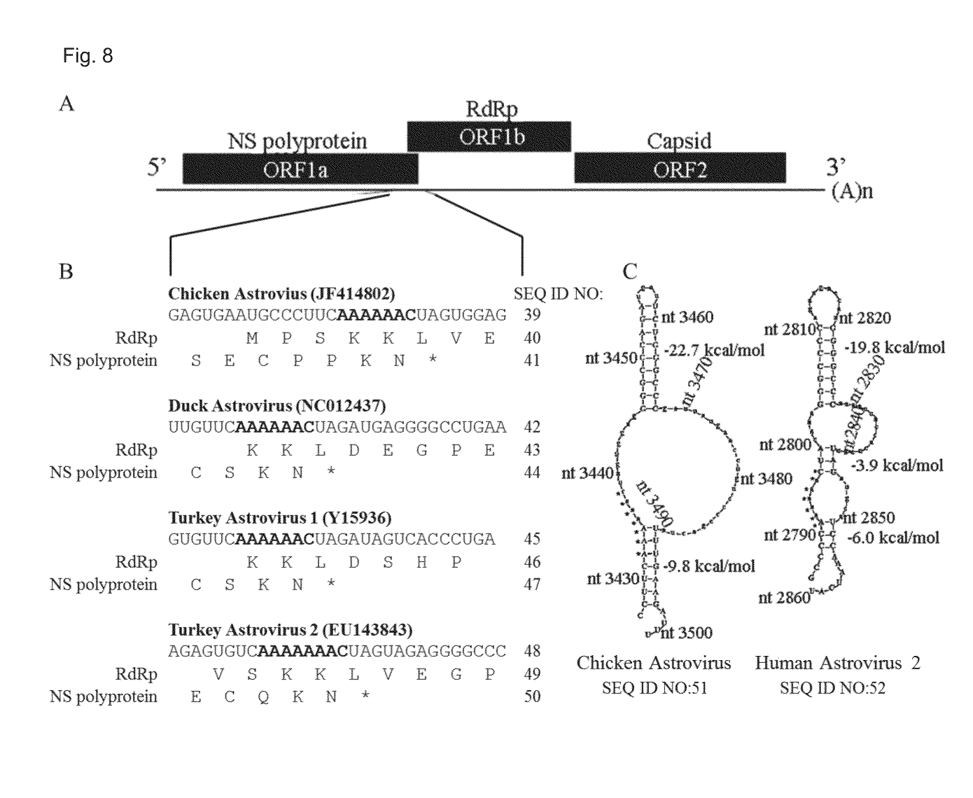

Determination of the full length sequence of a novel chicken astrovirus. The determination of the full length sequence, using the method of primer walking, resulted in RT-PCR fragments between 400 and 1200 bp in length. The determination of the extreme 5′-end was performed as previously described (Mundt and Muller, 1995, Virology; 209:10-18). The full length sequence of the virus genome is 7520 nucleotides (SEQ ID NO:1, see also Genbank accession number JF414802), not including the poly-A tail sequence. A schematic of the viral genome is shown in FIG. 8. The 5′- and 3′-noncoding regions were determined with 21 and 282 nucleotides, respectively. The data correlated with full length sequences from other avian astroviruses where the 5′ NCR was also a short sequence from 10 nt (Turkey astrovirus 1, (Jonassen et al., 1998, J Gen Virol; 79:715-718)) to 23 nt (Duck astrovirus (Fu et al., 2009, J Gen Virol; 90:1104-1108)). The 3′ NCR was also comparable in length to other bird astroviruses, with a range between 192 nt for turkey astrovirus 2 (Strain et al., 2008, J Virol; 82:5099-5103) and 305 nt (ANV1 (Imada et al., 2000, J Virol; 74:8487-8493)). The alignment of the nucleotide sequences showed that the first five nucleotides (CCGAA), located at the 5′ end, were highly conserved between all bird astroviruses (FIG. 7). In addition, this sequence motif was also observed in close proximity to the start codon for the ORF2, likely encoding the viral capsid protein. This feature has also been described for the duck astrovirus (Fu et al., 2009, J Gen Virol; 90:1104-1108) and turkey astrovirus 1 (Jonassen et al., 1998, J Gen Virol; 79:715-718) and 2 (Strain et al., 2008, J Virol; 82:5099-5103). In contrast, this motif was absent upstream of the proposed start codon for the capsid protein of ANV1 (Imada et al., 2000, J Virol; 74:8487-8493). Furthermore, turkey astroviruses 2, duck astrovirus, and the chicken astrovirus described in this article shared six homologous nucleotides at the very 3′ end (FIG. 7). Interestingly, when the ANV 1 sequence was also taken into consideration in the comparison of the 3′ end, the last three nucleotides were highly conserved between all astroviruses analyzed. The genome of the novel chicken astrovirus encodes three open reading frames (ORF), one protein each (FIG. 8) and follows the principal genomic structure for an astrovirus (Jiang et al., 1993, Proc Natl Acad Sci USA; 90:10539-10543). The first ORF (ORF1a) encodes for a protein of 1139 amino acids (aa), while the second ORF (ORF1b) encodes for 519 aa. ORF1a encodes for the NS polyprotein and ORF1b encodes for the viral RNA depended RNA-polymerase RdRp) as previously proposed (Jiang et al., 1993, Proc Natl Acad Sci USA; 90:10539-10543). The third ORF (ORF2) encodes, with 743 aa, the viral capsid protein (see also WO 2010/059899; U.S. patent application Ser. No. 13/107,140; and Sellers et al., 2010, Vaccine; 28:1253-1263). ORF1a and ORF1b are located in an overlapping position, while ORF2 is downstream from the ORF1b. Despite genomic similarities to other astroviruses described to date, slight differences were identified within the novel chicken astrovirus genome (SEQ ID NO:1). Although there is a potential ribosomal frameshift signal, consisting of a heptanucleotide (5′-AAAAAAC-3′), previously described (Jiang et al., 1993, Proc Natl Acad Sci USA; 90:10539-10543), the ORF1b contains its own start codon which makes this, by definition, a true ORF ((A) and (B) of FIG. 8). In addition, the proposed typical stem-loop structure was not present in the sequence determined, but rather a sequence was present in the proposed region which may form a strong hairpin structure with no possibility of forming a pseudo knot structure as proposed earlier (Jiang et al., 1993, Proc Natl Acad Sci USA; 90:10539-10543). The importance of this stem-loop structure is not clearly understood since changes in a model system in the structure did not abolish the expression of a pseudo ORF1a-ORF1b fusion protein but decreased the efficacy (Marczinke et al., 1994, J Virol; 68:5588-5595). On the other hand, deletion of the ribosomal frameshift signal sequence and also only a point mutation within the ribosomal frameshift signal sequence abolished the translation of the fusion protein in this model system (Marczinke et al., 1994, J Virol; 68:5588-5595), thus this sequence likely plays a central role for the translation of the fusion protein. Based on the data described in this example, it is possible that the ORF1b encodes the RdRp in the classical mode containing a start and stop codon. The possibility exists that in case of the nucleotide sequence here described for the chicken astrovirus, either a ribosomal scanning at the viral RNA with initiation at the start codon of ORF1b, or a that the ribosomal unit dissociates at the ribosomal frameshift signal sequence and reinitiates at the methionine of ORF1b or there is a ORF1b mRNA transcribed by an unknown mechanism. The latter possibility is rather unlikely due to the nature of the ORF1b encoding protein, the RdRp.

TABLE 4
Similarities of astrovirus amino acid sequences compared to sequences
of the novel chicken astrovirus

| | Chicken astrovirus | | |
|---|---|---|---|
| | ORF1a^{a} | ORF1b | ORF2 |
| Different astroviruses | (1139 aa) | (519 aa) | (743 aa) |
| Chicken astrovirus^{b} | N/A^{c} | N/A | 99-89%^{d} |
| Chicken astrovirus^{e} | 100% | N/A | N/A |
| Duck astrovirus 1 | 60% | 71% | 41% |
| | (YP002728001) | (ADB79812) | (YP002728003) |
| Turkey astrovirus 1 | 37% | 56% | 34% |
| | (Y15936) | (Y15936) | (Y15936) |
| Turkey astrovirus 2 | 46% | 71% | 44% |
| | (ABX46564) | (AAF60952) | (ABX46566) |
| Turkey astrovirus 3 | N/A | N/A | 38% |
| | | | (AAV37187.1) |
| Avian nephritis | 26% | 55% | 36% |
| virus 1 | (NP620617) | (BAA92848) | (NP620618) |
| Avian nephritis | N/A | N/A | 35% |
| virus 2 | | | (p) (ADQ43319.1) |
| Avian nephritis | 49% | N/A | N/A |
| virus 3 | (ACR56309) | | |
| Mink astrovirus | 26% | 33% | 32% |
| | (ADR65075) | (GU985458) | (NP795336) |
| Human astrovirus | 24% | 43% | 34% |
| VA1 | (ADR65075) | (AAY84778) | (ACX85476) |
| Ovine astrovirus | 23% | 34% | 32% |
| | (NP059945) | (NC002469) | (NP059946) |
| Bat astrovirus | N/A | 38% | 32% |
| | | (EU847155) | (ACN88712) |

^{a}Number of amino acids encoded by the each open reading frame.
^{b}Sequences published by Pantin-Jackwood et al. (Pantin-Jackwood et al., 2008, Avian Dis; 52:235-244).
^{c}No sequences available in the public database.
^{d}Percent homology to the amino acid (aa) sequence of the new chicken Astrovirus. Genbank accession numbers are listed in parentheses below the percentage similarity.
^{e}Sequence published by Baxendale and Mebatsion (Baxendale and Mebatsion, 2004, Avian Pathol; 33:364-370).

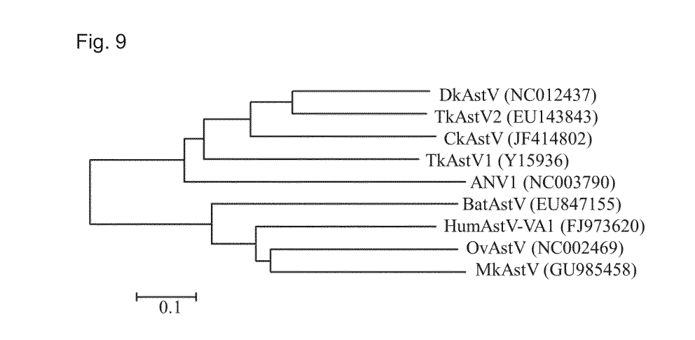

Analysis of the full length sequence with other Astroviruses. The nucleotide sequence obtained from the chicken astrovirus was compared in a phylogenetic analysis with other astroviruses using full length sequences. To this end, full length astrovirus sequences from several species (turkey astrovirus 1 (Y15936), turkey astrovirus 2 (EU143843), duck astrovirus 1 (NC012437), avian nephritis virus 1 (NC003790), bat astrovirus (EU847155), human astrovirus VA1 (FJ973620), mink astrovirus (GU985458), ovine astrovirus (NC002469)) were included in this analysis (FIG. 9). The sequences were aligned using the ClustalW program (available on the worldwide web at ebi.ac.uk/Tools/msa/clustalw2) and the multiple alignment obtained was analyzed using the program MEGA4.1. The neighbor-joining method and the minimum-evolution method were applied using 1000 replicates. The results of the neighbor-joining method clearly show that the novel sequence was significantly different (bootstrap value of 100) from other astrovirus sequences, including those described for ducks, turkeys, and chicken (FIG. 9) regardless of the algorithm used for the phylogenetic analysis. To further analyze the relatedness of this virus to other astroviruses, the amino acid sequences of all three in vitro translated ORFs were compared with published sequences (Table 4) using the pBlast search option in the NCBI database with one exception—the partial amino acid sequence for the ORF1a protein for a previously published chicken astrovirus (Baxendale and Mebatsion, 2004, Avian Pathol; 33:364-370) was taken from the publication and compared using the DNASTAR program package since this sequence is not available in the NCBI database. A 100% identity with a 99 aa partial ORF1a sequence was observed which has been described for a chicken astrovirus isolated in Europe (Baxendale and Mebatsion, 2004, Avian Pathol; 33:364-370). A high similarity (99-89%) was observed with partial sequences of the capsid protein of previously described chicken astrovirus sequences obtained from US field samples (Pantin-Jackwood et al., 2011, Arch Virol; 156:235-244). Interestingly, the overall amino acid sequences indicated that the most similar relative to the novel chicken astrovirus was a recently described duck astrovirus which caused a fatal hepatitis in ducklings (Fu et al., 2009, J Gen Virol; 90:1104-1108), followed by turkey astrovirus 2 (Strain et al., 2008, J Virol; 82:5099-5103; Tang et al., 2005, Avian Dis; 49:514-519) and 1 (Jonassen et al., 1998, J Gen Virol; 79:715-718). A turkey astrovirus 3 capsid protein sequence showed a 38% identity (Tang et al., 2005, Avian Dis; 49:514-519). Surprisingly, the deduced amino acid sequences of all three proteins of the novel chicken astrovirus similarly showed a low similarity to the corresponding sequences of ANV1 (Imada et al., 2000, J Virol; 74:8487-8493) and to the capsid protein sequence of ANV2 in addition to the expected lack of similarity observed with the mammalian astroviruses, such as ovine, mink, human, and bat astrovirus (see Table 4). This data indicates the high degree of variability between astroviruses isolated from the same species. In addition, the similarity to the RdRp amino acid sequence was always higher likely due its nature as a functional enzyme responsible for the replication of the virus genome. RdRp sequences appear over-represented in the NCBI database, likely due to their highly conserved nature, compared to the few sequences available for the remaining regions of the genome. This region also serves as a target for the development of diagnostic tools (Pantin-Jackwood et al., 2008, Avian Dis; 52:235-244; Smyth et al., 2009, Avian Pathol; 38:293-299; and Todd et al., 2010, Avian Pathol; 39:207-213).

Determination of the full length sequences of viral genomes will certainly provide the basis for a better understanding of the biology of particular virus. Data obtained in this study support the need to determine the full length sequence of novel viruses since due only to the availability of the full length sequence was evidence found to suggest that this particular astrovirus may employ a different replication strategy than what has been described for other astroviruses. Based on the findings of this example, experiment against the proteins encoded by ORF1a and ORF1b are under way to determine the proposed alternate mechanism for astrovirus replication.

Example 3 has also published as Kang et al., “Determination of the full length sequence of a chicken astrovirus suggests a different replication mechanism,” Virus Genes, 2012 February; 44(1):45-50 (doi: 10.1007/s11262-011-0663-z, Epub 2011 Aug. 31), which is hereby incorporated by reference in its entirety.

### Example 4

### Full Length Genomic Nucleotide Sequences and Amino Acid Sequences of ORF1a, ORF1b, and ORF2

Full length genomic nucleotide sequence of a chicken astrovirus (CkAstV) isolated from the intestinal content of RSS affected chickens (“gut”). Also found as Genbank accession number JF414802 (version JF414802.1, Feb. 15, 2012), which is hereby incorporated by reference in its entirety (SEQ ID NO:1):

(SEQ ID NO: 1)
CCGAAAGCGAGGGTGTGGGCGATGGCCCAGGCCATGGCGGGGGCTTTTTCAAGCC
TTAACAGGCGAGAAGACCGTAAGTCGGCCCAAGTAACAGCTGGGCTTGACAAGGT
CTTCTCGTTCCAGGGCGTGCACGAACTTTTTGTAAGAATGCGTGCGCTGTCCGGAA
CAACTACAGCATGGAAAGCCCTGATGAATAGTGAAGCTGTGTATATTAAAGATGT
AAAAACGGCTTTTGGCGCGAATGGTTCCCAAATTGGCTTCTTCTTTGCTGAGACAC
CCACAACACCAACTTGGTCTCCAGATGCTGGGATTGCTATATTGACTGAAGGTGAA
AAAACTTGTCTTGCAGCACAGCAGGCAAGGGATCTGCGCCTGAAAGCTTCACTAA
GCACAAACAGCTCTCTGGTCCACCAGATTATGGAGAAGACAAGAGAGGCCAAGGA
GAAAACGAAACAACTCGAAGAACTCCAGCATCGTATTGACAATATGGTGGATACT
AATAAAGTCTTGTATCAACGCATGGAACAGCGGCATCAGGAGAAGTTGGAAGCCC
TTGGTGAGAAGATCAGTAAACTTAGATATGATAACCATCAGTGGTTCATGACTTGT
GAGAAAAAGGATGAGGAAATAGCAAACCTCAAGCGTCAACTTGAAACTAAAAAG
AGTGGCTATAAGAAAATGGCCTGGGACGCAGTTGCTTGGTTTGTGCTAGCTATCCT
CTTATTCAGTTTTTTCTCTGTGAGTGAAGGTGCCACACCTAATGGAACTTTACCAAT
TAAGGATGTTAGTTGGAACTTTGATGACGTGGAAAAAACTTGCATGAAACCTGATT
TTGGCTGTTTGGTTATGGACACCTGGCTACCACATCCCGTCCTAACGTTTGAGGACT
TAATGTCTAAGTGCTACAATACTCATGGGAATGTCATACCTCGTAGTGCTTTTAATA
GTGAACAACTCTTGTATGACTGCACAAAAACGGCACACTATTTTAACGATGGGCAT
GACTACATTGAAAATTACCACTGGTGTGAGAAAAGGTTAGCAACTCTGGTGGCAG
CAAACTGTCAAGGAGATAATGGTGTTGATAAGATCTATACGCAAGTTGTCGAGGCT
GTGGCAGCAAGTAGGAAATTCTTTCAGAAAATGGCACTCTATAAGCTTGATGTTTG
GATACTAGCTATTTTTAGTATTGTGTTAGCGGGAAATAAGGAAAAAATAGTAAAAT
TAACCCCTTTTATAGCGTTAGGCTGGTGGTTTAATTTACCTATATTCCTACTGAGTA
CCGCTGTTAACTTCTTTCCTACAATGGCCTTGCCTTTTATTGCGTTCCAGATTCTCCA
ACCAGGGTTTCTAGTTGTAACAGCCTTTACATTGTGGTTGACACTAACACTTGCTGC
TTTCTTTTGGAATGATGGAATAGCCATCTTAGTTGAGACGTCATTTGCCTTGTTCTA
CACGATCTTGTTTTTTGTTTGGTCGATGGCAATGACAGTCTGTGTTAGTTTACAGTT
GAGTTTAGCGTACCAAATACTCTTGTTCTGTGTTTCGCTTAGTGTGTATTGTGGAAC
AAAGTTTGCGTGTAGTCAGGTTGTTATAACAAATCCTGATGGAACAACAGAAAAA
GTTTCAAGAGTAGGTAAGGTTAGGAAAGCAGCCTTTCAACAGTGTAAGGGTGTATT
GACCTTCTTGCAAACACGGGGGATTATTCCATCCACACCTGTTAAAACTAATAGTG
TAGTCGTTATTACAGGAAAGAATGGTGCAGGTACTGGATTTAGGTTTATGAATTAT
ATTGTCACAGCAGGCCATGTTGTCACCGGTAGTGAATGGGCAACAGCTAAATTCGG
TGATGTTTCAGTTAAAATTAAGAAGGAAAAAGAAATTGACATGTGTGAGTGCCCTG
ACACACTTGTCCTTTTTAAATTACCAAAGGAATTGCAGAGTGTTAAACCATTAAGA
TTAGCCCAAAATATAAAGTCAAATTATATGACATTGCACGGGTTTTGTCCAAATTT
TGTCAACCCTGTTAGTTTTACAGGCTGGTGTACTATTGATGGACCTTGGCTAAACA
ATGCTTTTAATACAACTTTTGGTAATAGTGGTGCTCCATACTGTGATCCCGATGGGA
AGCTTGTTGGTATTCACCTAGGAACCCAAGGAGTAACTTCACAAGGTTTTGTTATA
TGTGATACTCTTAGAAAGCAATTTGAAACACACTATCAGTGTTCGTGCAGGAGAGA
AGAGTGTGATCAACAACCTGCACCGGCACCACCTGTGCAGTTCGACTATGAAGAAT
TTTTGTCTAAAGTTATAACAGGAACAAAGGTTTCCCATCAGGCCATACTGAAACAA
CAAGAAGAAATGTTAGAGCAAATTCAGCTTGTAACAAAGATTCTTCAGGAAAATG
GCTTGTTAGCTGAACAGAAGAAGAAAGGAAAAACAAAAAGAACTGCTCGTGGTGC
TAAGGCTACAATGACAAAGAAATACCTTTCCAAAGGACACTTCATGAAAATGAAA
ATGTTAAGTGAAGAGGAGTACCAAAAATTGGTGGATGAAGGCTTCACTGCCGATG
AGATTAAAGAAGTTGTCAACAACCTTCGGGAGCAAGCATGGCTTGAATATTGTATT
GACAATGATATTGATGATGAGGGAGCAGAAGATTGGTATGATCAGATGTTAAATG
ATGAGGCAATTAATGACCAAATTGATAGGGAAATTGAAGCAAAAATGGAAGATGA
AGGCTTTTATCAATCAAAAGCCAGAGAAACTTTGGCATCACAGTGCAAAGATAAA
AAACGCAAAACTTTTGCTGAACAGGCCCTTCTGCATATTATAGATCTTAAACCTCA
TAAGGTTAGGACAGTTAAGGTTGAGGTTCAGGACGAGAGTGCCAACCAACTCAAA
AAGGTCTTTAAGAGAATGGTCAAAGACGAGGAAGTTGAACAGGGGCAAACGAAG
GCCTTCTTTTCTTCAGGTGATGATATAGAATATTTTGAAAATCGTGATATTGATTGG
AAAAAATTGGAAATGCCAAAATTACAGGATGAGGTTAAATTTGAGACCATCACGC
GCAATGGCATAACTCAAATTTCTACTGGTGAGGATAATAAGAAAAACATCCTCAA
AGAAAAGGTCACAAATATACCACAACCGTTGACACCAGTTCCACCGTTTACGGAG
AATAATCCGGACACTAAGCCTAAAAAGGAGCAGGTTCTTGAACAACGGAAGAGGG
TTTGTAGGTCATGTGGTAGCGACAAACCCCACAACTTCGTTGTGTGTAAGAAAAAG
AATGAGCTCTTGTTCTGTGTCTGGTGTGGTATAGTGCACTCTGAGAACCAAGGCCA
CTCACGGAAAATCGAATGCCCTAAATGCAACAAGGCATTCTCTGGGATAGAGGGT
CTTGAACGCCATGCGAGTGAATGCCCTTCAAAAAACTAGTGGAGGGCCCAGATGA
TTCTGGGCCCGATTATGAACCTGTCCCCGACTATTTGAAGATTTTTTCTTGGGAAGA
TGACTTGCTACCTCCAATTGGAAAAGAAGCTCTACCTGAAAATGTTATACTTCTTG
GCCATATTCCGGTTGATAAATTGGTTTCTAGAACAAAGAAGGTAACTGATCCACTA
CTTGGAGTTATCACTTCTTGGAAACAGGATGAATATGATAGTACCACATGGACTGT
TAAGGCGTACACTAAAATGTTTGAGAAATTCTTCTATAAAGAACCATCAGATTTTG
TGAATAATGAGCCAGAGCTCACCATCCTATGTGATAGGGTGGTGCTTGATGAACAT
GATTATATGGCAAATAGTTCTATGACGCCTATTACAGCGACTATAAAAAATGTGGA
CTCAACACCTGCTTATCCAAAATTCCAGGAGTTTGATACCGAAGAAGAATATCTTA
CCCGTTGTGGATGGAGTGAATACCTCGATGTCATTAAGAACAAAGAAACTCTCAAC
CATAGGCCATTATGGTGGTGCTTCCTTAAAAACGAAGTTCTCAAGAAAAAGAAAAT
TCAAGAGAATGATATTAGGATGATTTTATGTACAGATCCAGTCTTCACTCGAATTG
GAGCAATGTTTGATCAGGACCAAAATTCAAAAATGAAAAATATGACAGAAACGAG
GGCCGCACAAGTGGGCTGGACCCCCTTCTTTGGTGGACTTGACCAGCGAATGCGGA
GGTTGGAGAAGATTGAAAACGCCCAGTTTGTAGAAATGGATTGGACCCGCTTTGAT
GGCACTATTCCAAAGGCGCTTTTTTGGCGCATCCGGCAGATCAGATTCTTTTTCCTA
GCGCCCCGGTATAAAACCGCGGCTAACAAGGAATTGTTTGATTGGTACACTAAAA
ATCTGCTGGAGAAAATAATTCTCTTACCAACTGGAGAAGTGTGTCAGATAAAAAG
GGGTAATCCTTCTGGGCAGTTCTCCACCACAGTTGATAACAATATGTGTAATGTGT
GGTTAACTACCTTTGAAATAGCATGGCTCCACCGCAAACAGAAGGGCAGACTCCCC
ACTCCCACTGAGTTGAGGAAAAATCTTAAGTACATTTGCTATGGTGATGACAGGCT
CTTGGCAGTTTCAAAGGATTTTGTGGTCTATGAGCCTGACACTGTTGTTAAAATGTA
TGCAGATGTATTTGGGATGTGGGTGAAACCAGAAAATGTAAAAGTTAGAGATAGC
CTAGTTGGTCTCTCGTTCTGTGGGATGACAATTATCAAAAATTCGAACAACCGTTA
TGTAGGGGTTCCCAATGTAAATAAAATTCTTTCTACTCTCAGTACTCCTACAAAAA
GGCTTCCAAATATCGAGGCCTTGTGGGGGAAATTGATCTCACTGCGGATCCTTTGT
GAGAATGCTGATCCCGACGTCAGGGACTACTTAGATAAACAGATCCACTGCGTCG
AGGAATATGCTGCTGCTGAAGATATACAGTTACCAGAAGTCGGGCCCGACTTCTTT
CAGAAAATCTGGTAGAGGGATGGACCGAAATAGAGCAGCATGGCCGATAAGGCTG
GGCCGCAGAAGAAGAGGGCATCTAGGCGCGGGCGTGGCCGCTCTCGCTCAAGGTC
ACGCTCACGCTCTCGATCAAGAAATCGTGTCAAGAAAACAGTCACGATCGTTGAA
ACAAAAAAGACCCCAAGTAAGTCAATCCTGAAAAAGGAGTTGGATAATCATGAGA
GGAAAGATAGAAAAAGGTTCAGAAAATTGGAAAAGAAATTAAATGGACCAAAAA
TTCATGATCGCATGGCGGTTACAACCACTCTTGGTGTTCTCACTGGCAATTCTGACA
ATAATTTGGAAAGGAAGATGAGGGCATTGCTCAACCCCTTACTCTTAAAATCCCAG
AACACTGGAGCATCGGCATCACCACTCTCTCTTAGAGCCTCACAGTATTCAATGTG
GAAAATACAGCGGTGTGTTGTTAAATTTGTACCGCTGGTGGGAGCAGCAAATGTTG
CTGGAAGTGTGTCATTTGTGTCGTTGGACCAAGATGCAACATCCTCTCAACCTGAA
TCACCTGATACTATAAAGGCGAAAGTGCATGCCGAGGTGTCCATTGGCCAGAGGTT
CAACTGGAATGTGCAATCTAGATACCTTGTTGGACCGCGGTCTGGCTGGTGGGGTA
TGGACACCGGAGAGTCACCAACTGACACAGTTGGTCCAGCACTTGATTTTTGGAAT
TTATATAGGACAGTAAATACTCTTCAGACTGGAACAACATCACAAGTCTACACTGC
TCCATTATTCTCTATAGAAGTGTTCACCGTCTATGCATTCTCGGGATACGAGCCTAA
ACCTGCCCTTGCTACAATGACAAATTCTACTTTTGAAAGTCAGCAGGGGGTAACCA
TAACAAATGGTTCCAATGGTGAACTTCTTCTTAACGTTCCACAAAGGTCGGCATTT
GCAGAAAGGCTCCGCGAAAAAGAGACACCACAGCGTGCGCAAAATCAAACCGGC
GGGGTTGGAGAGGTGCTGTGGGCTGTAGCATCTGGTGCAGTGGAAGGGGCTGCAG
AAGCGTTGGGCCCGTGGGGCTGGCTACTAAGAGGTGGCTGGTGGGTCATTAAAAA
ATTATTTGGTAGAACCGGAGAAGATGCAAATGATGTGTACGTGATGTATTCATCAA
TTGAAGATGCAAACAAAGACAGTAGAATATATCAAACTGTCACTGGATCGGTGCA
AATACAACAAGGCCCACTCGTTCTAACACAAATCTCATCGCCGAATGTGAATACAT
CCGGAGGGGTTGTTCAGGTAAATTCAACCACTCCAAATGACTACTTGCCCCTCTCT
CAAGAAAGTTATGCAGAGACACCATTGAAAAAATATGTACTTTATGACAGCACCG
GGAACCCCGTTGATAGCAATATGAGCCACACCATGAGGATAACAGGGTATCCTGA
GTCAAAACTAGTGACATCAAGCTCAGTCTGGCTCGGTACAACTGGTAAGAGCGTAC
AATCAACTAAATGGCTGATGTCCGATTACACAAATACAGGGGTCATATTTGGTTTT
CCTTATACGAGCGCACCACCGGGAGCAACTGTCGGCAACATTGGTGTCATTCATAC
TGCAAAATCATTAATTAAGACAATCAGATACAGAAGACAAAACCATCTTCCAACA
ACACCTTTTGAATCTTCCCTGATACCGTCAGCGTCAAAAGGACCCAGTCAAATGCT
AGGATGTTTTGACACCCCATACGTATGGTGTAGAGTTTGTGATAATACATGCTCCA
CCAAGCCTACTGATGGTGCAGTTACACAGAGGTACAATGCATGGGGCCTGCTGGTG
GTTAGCCTTGCCCATGATAAGGTGTATGTACTATCAGGCTATCCAAATTCACAAAC
AGCAGTACCAGTGCAACAATTGGTTTGGGATACTTTTGACTGGGATGCAAATTTTT
CCACTGGCAGAATTTATAGCGCAGTATGGCCAGGTGAAGATGGTGCTGAACAGGA
AGGTTCAGACACTGATGATGCTGACTCTGACATTTCCAGTCTTTTTGACCCAATGA
ATGAGGTGGAAAAGGACTTCCATTTCCAGTGTAGTCTCAAAACTTCTGACTACTTA
AAAGAGGAGGCTGACTTTTGGAAAGCAAAGGCGCAACAGCTACTCATGGAGAAGG
CAATGGAAAAACCAAGTGCTAATCCTCCTCTTGTCCGCTTCGAGAAGGGTGGACCT
GAGCAGCAAAAACAACCTGCTAGCAGCCGCGGCCACGCCGAGTAGGATCGAGGGT
ACAGCTGCACCTCCTCAATGGAGTTTTTATGCCGTAATCAGGCTTTTCTCCATTCAA
AAATTAAGGCACCGGGGCCACGCCGAGTAGGATCGAGGGTACAGTGCCGGGTTGA
CCTCACCTTAAAGAGGCGTCCGCCGGTATGGAAATCACCATGCTGGAGTCTGGTTA
AGATCACAGACAATCACTCTGTGTGTAAATCAGGCTTGTCGGGCGGTTTTGGAAAC
GTAGGTTTTTAAAACCAATTTGATTTTGAATTAAATTAATTGGCATTTAAAAAAAA
AAAAAAAAAAA

Full length genomic nucleotide sequence of CkAstV isolated in cell culture (“CkAstV-p5”) (SEQ ID NO:2):

(SEQ ID NO: 2)
CCGAAAGCGAGGGTGTGGGCGATGGCCCAGGCCATGGCGGGGGCTTTTTCAAGCC
TTAATAGGCGAGAAGACCGTAAGTCAGCCCAAGTAACAACTGGGCTTGACAAGGT
CTTCTCGTTCCAGGGCGTGCACGAACTCTTCGTGAGAATGCGTGTGCTGTATGGAA
CATCTACAGCATGGAAAGCCCTGATGAATTGTGAAGCTGTGTACATTAAAGATGTT
AAAACGGCTTTTGGCGCGAATGGTTCCCAAATTGGCTTCTTCTTTGCTGAGACACC
CACAACACCGACTTGGTCTCCGGATGCTGGGATTGCTATATTGACTGAAGGTGAAA
AAACTTGTCTAGCAGCACAGCAGGCAAGGGATCTGCGCCTGAAAGCTTCGTTAAGT
ACAAACAGCTCCCTTGTCCACCAGATTATGGAGAGAACAAGAGAGGCCAAGGAGA
AAACGAAACAACTTGAAGAACTTCAACAACGGATTGATAATATGGTGGACACTAA
TAAGGTTCTTTACCAACGCATGGAACAGCGGCACCAAGAAAAATTGGAAGCTCTT
GGTGAGAAAATTAGTAAACTTAGGCATGATAACCATCAGTGGTTCATGACTTGTGA
GAAAAAGGATGAGGAAATAACAAAACTTAAACATCAACTTGAAACCAAGAAGAA
TGGCTATAAGAAAATGGCCTGGGATGCAGTTGCTTGGTTGGTGTTAGCTATCCTTTT
ATTCAGCTTTTTCTCTGTGAGTGAAGGTGCCACACCGAATGGAACCTTACCAATTA
AGGAAGTCAGTTGGAATTTTGATGATGTGGAAAAAACCTGTATGAAACCTGACTTT
GGCTGCTTGGTTATGGATACATGGTTACCACATCCTGTTTTAACATTTGAGGACCTA
ATGTCTAAGTGCTACAATACTCATGGGAATGTCATACCTCGTAGCGCTTTTAGTAG
TGAACAACTCTTGTATGACTGCACAAAAACGGCACACTATTTTAACGATGGGCATG
ACTACATTGAAAATTACCACTGGTGTGAGAAAAGGTTAGCAACTCTGGTGGCAGC
AAACTGTCAAGGAGATAATGGTGTTGATAAGATCTATACGCAAGTTGTCGAGGCTG
TGGCAGCAAGTAGGAAATTCTTTCAGAAAATGGCACTTTACAAGCTTGATGTTTGG
ATACTAGCCATTTTTAGTATTGTGCTAGCGGGAAATAAGGAAAAAATAGTGAAATT
GACCCCTTTCATAGCGTTAGGGTGGTGGTTCAATTTACCTATATTTCTATTGAGTAC
CGCTGTTAACTTCTTTCCTACAATGGCCCTGCCTTTCATTGCGTTTCAAATTCTTCAA
CCAGGGTTCTTAGTTGTAACAGCCTTCACATTGTGGTTGACATTGACGCTTGCTGCA
TTCTTCTGGAACGATGGAATAGCTATTTTGGTTGAGACATCATTTGCTTTGTTTTAT
ACAATCTTGTTCTTTGTCTGGTCAATGGCAATGACAGTTTGTGCTAGCTTGCAATTG
AGCTTAGCATATCAAATACTTTTATTTTGTGTTTCGCTTAGTGTATACTGTGGAACA
AAGTTCGCTTGTAGTCAGGTTGTAATAACAAATCCTGACGGCACAACTGAAAAAGT
CTCCAGAGTAGGAAAAGTTAGGAAAGCAGCCTTCCAGCAGTGTAAGGGTGTGTTG
ACTTTCTTGCAAACACGGGGAATTATTCCATCCACCCCTGTTAAAACTAATAGTGT
AGTTGTAATCACAGGAAAGAATGGTGCAGGCACTGGCTTTAGATTTATGAATTATA
TTGTTACAGCAGGCCATGTCGTCACCGGAAGTGAATGGGCAACAGCTAAATTTGGT
GACGTTTCAGTTAAGATTAAGAAGGAAAAAGAAATTGACATGTGTGAATGCCCTG
ACACAATTGTTCTTTTTAAATTACCAAAAGAACTGCAGGGAGTTAAACCATTGAGA
TTGGCGCAAAATGTAAAATCAAATTACATGACATTACATGGTTTTTGCCCTAACTTT
GTTAACCCTGTTAGCTTTACTGGCTGGTGTACCATTGATGGGCCTTGGTTGAACAAT
GCTTTTAATACAACTTTTGGCAATAGTGGCGCTCCTTATTGTGACCCCGATGGTAAG
CTCATTGGCATTCATTTAGGAACTCAAGGAGTAACTTCACAAGGCTTTGTTATAAG
TGATGTCCTTAGGAAACAGTTTGAAACACATTATCAGTGTTCGTGTAGGAAGGAGG
AAAGTGTCCAACAACCTACACCTGCACCGCCTGCGCAGTTTGATTATGAAGAGTTC
TTGTCTAAGGTTATAACAGGAACAAAAATTTCCCACCAAGCCATATTGAAACAACA
AGAGGAAATGTTAGAACAAATTCAGCTTGTAACAAAGATCCTTAAGGAAAATGGC
TTACTGGCTGAACAGAAGAAAAAAGGGAAAACTAAAAGAACTGCTCGTGGTGCTA
AGGCTACAATGACAAAGAAATATCTTTCTAAGGGGCACTTTATGAAAATGAAAAT
GTTAAGTGAAGAGGAATACCAGAAATTGGTGGATGAGGGCTTCACTGCTGATGAG
ATTAAGGAAGTTGTTAACAACCTTCGGGAGCAAGCTTGGCTTGAATATTGCATTGA
CAATGATATTGATGATGAAGGTGCAGAAGATTGGTATGACCAGATGTTGAATGAT
GAGGCAATTAATGACCAAATTGATAGGGAAATTGAAGCAAAAATGGAAGATGAGG
GGTTCTACCAAGTAAAAGCTCGAGAAACCCTAGCATCACAATGTAGAAACAAAAA
ACGCAAAACTTTTGTTGAACAGGCTCTTTTGCATATTATAGATCTTAAGCCTCATAA
AGTTAGGACAGTTAAGGTTGAGGTTCAGGATGAGAGTGCTAACCAACTTAAAAAG
GTCTTTAAAAAAATGGTCAAAGACGAGGAGGTTGAACAGGGGCAAACGAAGGCCT
TCTTTTCTTCAGGTGATGATATAGAATATTTTGAAAATCGTGACATTGATTGGAAA
AAATTGGAAATGCCAAAATTACAGGATGAGGTTAAATTTGAGACTATCACGCGCA
ATGGCATAACTCAAATTTCTACTGGTGAGGATAATAAGAAAAACATCCTCAAAGA
AAAGGTCACAAATATACCACAACCATTGACACCAGTCCCACCATTTACGGAGAAT
AATCCGGACACTAAGCCTAAAAAGGAGCAAGTTCTTGAACAACGGAAGAGGGTCT
GTAGGTCATGTGGCAGCGACAAACCCCACAATTTCGTTGTGTGCAAGAAAAAAAA
TGAGCTCTTGTTCTGTGTCTGGTGTGGTATAGTTCACTCTGAGAATCAAGGCCACTC
ACGGAAAATCGAATGCCCTAAGTGCAACAAAGCATTTTCTGGGATAGAAGGTCTT
GAACGCCATGCGAGTGAATGCCCTCCAAAAAACTAGTGGAGGGCCCAGATGATTC
TGGGCCCGATTATGAACCTGTTCCCGATTATTTGAAGATTTTTTCTTGGGAAGATGA
CTTGTTACCTCCAATTGGAAAAGAAGCTCTACCTGATAATGTTATACTTCTTGGTCA
TATTCCAGTTGACAAATTAGTTTCTAGAACAAAAAAGGTCACCGATCCACTACTTG
GTGTTATTACGTCCTGGAAACAGGATGAGTATGACAGTACTACATGGACAGTGAA
GGCGTACACGAAGATGTTTGAAAAATTTTTTTATAAAGAACCATCTGACTTTGTGA
ACAATGAGCCAGAATTGACCATCCTTTGTGATAGGGTGGTGCTTGATGAGCATGAT
TATATGGCTAATAGTTCTATGACGCCTATAACAGCAACTGTCAAAAATGTAGACTC
AACACCAGCTTATCCAAAATTTCAGGAGTTTGATACTGAGGAAGAATATCTCACCC
GTTGTGGATGGAGTGAATACCTTGATGTTATTAAGGACAAAGAAACTCTCAACCAT
AGGCCATTATGGTGGTGCTTCCTTAAAAACGAAGTTCTCAAGAAAAAGAAAATTCA
AGAGAATGACATTAGGATGATTTTATGTACAGATCCAGTTTTTACTCGAATTGGAG
CAATGTTTGATCAGGACCAAAATTCAAAAATGAAAAATATGACAGAAACGAGGGC
CGCTCAAGTGGGCTGGACCCCCTTCTTTGGTGGACTTGACCAGCGAATGCGGAGGT
TAGAGAAGATTGAAAACGCCCAGTTTGTAGAAATGGATTGGACCCGCTTCGATGG
CACTATTCCAAAGGCGCTCTTTTGGCGCATCCGGCAGATCAGATTCTTTTTCCTAGC
GCCCCGGTATAAAACCGCGGCTAACAAGGAATTGTTTGATTGGTACACTAAAAATC
TGCTGGAGAAAATAATTCTCTTACCAACTGGAGAAGTGTGTCAGATAAAAAGGGG
TAATCCTTCCGGGCAGTTCTCCACCACAGTTGATAACAATATGTGTAATGTGTGGC
TAACTACCTTTGAGATAGCATGGCTCCACCGCAAACAGAAAGGTAGACTCCCCACT
CCCACTGAGTTGAGGAAAAATCTCAAGTACATTTGTTATGGTGATGACAGGCTCTT
GGCAGTTTCAAAGGATTTTGTGTCCTACGAGCCTGACACTGTTGTTAAGATGTATG
CAGATATCTTTGGGATGTGGGTGAAACCAGAAAATGTGAAAATCAGAGATAGCCT
AGTTGGTCTCTCGTTCTGTGGGATGACAATTATCAAAAATTCGAGCAACCGTTATG
TAGGTGTTCCCAATGTAAATAAAATTCTTTCTACTCTCAGTACTCCTACAAAAAGG
CTTCCAAATATCGAGGCATTGTGGGGGAAATTGATCTCACTGCGGATCCTTTGCGA
GAATGCTGACCCCGACGTCAGGGACTACTTAGATAAGCAAATCCACTGCGTCGAG
GAGTATGCTGCTGCTGAAGAAATACAGTTACCAGAAGTCGGGCCCGACTTCTTTCA
GAAAATCTGGTAGAGGGATGGACCGAAATAGAGCAGCATGGCCGATAAGGCTGGG
CCGCAGAAGAAGAGGGTATCTAGGCGTGGACGTGGCCGCTCTCGTTCAAGGTCAC
GCTCACGCTCTCGATCAAGAAATCGTGTCAAGAAAACAGTCACGATAGTTGAAAC
AAAAAAGACCCCAAGTAAGTCAATTCTGAAAAAGGAGTTGGACAATCACGAGAGG
AAAGACAGAAAAAGGTTCAAAAAATTGGAAAGGAAACTAAATGGACCAAAAATT
CATGATCGCATGGCAGTTACAACTACTCTTGGTGTTCTTACAGGTAATTCTGATAAT
AATTTAGAGAGGAAGATGAGAGCCTTGCTTAATCCACTTCTGCTAAAATCCCAGAA
CACCGGAGCTTCAGCGTCACCACTCTCTCTTAGAGCATCACAATATTCTATGTGGA
AAATACAGCGGTGTGTTGTTAAATTTGTGCCGTTGGTGGGTGCAGCAAATGTTGCT
GGAAGTGTGTCATTTGTATCATTAGACCAGGATGCAACATCATCCCAACCTGAATC
ACCTGACACTATAAAAGCGAAGGTACATGCAGAAGTCGCAATTGGGCAGAGGTTC
AATTGGAATGTGCAATCCAGGTACCTGGTTGGACCCCGTTCTGGTTGGTGGGGTAT
GGATACTGGTGAGTCACCAACTGATACGGTTGGACCAGCTCTCGACTTCTGGAACC
TCTACAGAACAGTAAACACACTCCAGACTGGCTCAACATCACAGACGTACACTGC
GCCATTGTTTTCTGTGGAGGTCTTCGCAGTGTATGTGTTCTCAGGATATGAGCCTAA
ACCTGCTCTTGCAACCATGACAAATTCAACTTTTGAAAGCCAGCAGGGAGTAACTA
TAACGAATGGTTCTAATGGGGAACTTCTGCTTAATGTACCAAAGCAGTCAGGTCTT
GCCGAGCGACTACGTGAAAAAGAAACACCGCAACGAGGCCAAAACCAGTCAGGC
GGAGTTGGGGAGGTTCTATGGGCAGTTGCATCGGGCGCTGTGGAAGGAGCTGCAG
AAGCACTGGGCCCATGGGGTTGGCTACTTCGGGGTGGCTGGTGGGTTATTAAAAAA
CTGTTTGGGCGGAGCGCTGAGAATGCTAATGATGTCTTTGTAATGTATTCATCTATT
GAGGATGCTAATAAAGATAGCAGAATTTACCAGACAGTCACAAGCGCAGTACCAG
TCCAGCAAGGCCCACTAGTCCTTACCCAAATTTCATCACCCAATGTCAACCAGGCC
GGTGGAGTAGTACAAGTGGGATCGTCACTTACAACTGACTATCTGCCACTTTCCCA
GGCGCAAGTCCCATTTTTGGAAAATGTCCTTTATACTAACACTGGTCAGCCTTTGAC
GTCCAACAAGAGTCATACAATGAGGATAACTGGATTTCCGGCCTCAAAACTAGTA
ACATCGTCATCATCTCAATGGCTGGGTACTGCTGACAAAAGTGTTCAAGCGACAAA
ATGGCTTATGTCTGACTACACCGATACAGGAGTGATCTTTGGTTTCCCCTACTCTAA
TGATCAACCAGGAGAAACATTGGGCAATATCGGTGTGATACACACATCAAAGTCG
CTTTTGAAAACAGTGACATCACGGCACCAGCGTAGGCTGAGCATGGCACCACTAAT
TTCAACCCCAATACCATCGGCTTCGAGGGGGCCAACACAGATGCGTGGATGTTTCG
ACACCCCCTACTATTGGATAAGGGTTTGTGATAACACATGTTCTAATAAACCCACA
AACGGAGCCGTTACACAGCGGTATAATGCATGGGGTGTCATGGTGGTGAGCGTAG
TTCACAATAAAATCTATGTTCTAGCCGGTTATCCTGATGGCCAAAATAGAGTACCA
CAACAGCAAATGGTATGGGACACGTTTGACTGGGATGCCACATTTACCACTGGCAG
GATCTATTCCTCAACATGGCCAGGCACTGAGGAAGAAGTTGAAGATGAGACTGAT
GCTGAGTCTGATATTTCTAGCCTGTTTGATCCAGTGAATGAAGTGGAGCAGGATTT
TCATTTTCAGTGTAGTCTTAAGACATCAGACTACCTAAAAGAGGAGGCGGATTTTT
GGAAGGCAAAGGCACAACAGTTACTAATGGAAAAAGCAATGGAAAAATCAGCTTC
CAATCCTCCTCTTGTCCGCTTTGAGAAGGGCGGACCTGAGCAGCAAAAACAACCTG
CTAGCAGCCGCGGCCACGCCGAGTAGGAACGAGGGTACAGCTGCACCTTCTCAAT
GGAGGTTTTATGCCGTAATCAGGCTTTTCTCCAAAATTAAGGCACCGGGGCCACGC
CGAGTAGGAACGAGGGTACAGTGCCGGGTTGACCCTACCTTAAAAGGGCGTCCGC
CGGCATGCAATCACCATGTCGGGGTCTGGTTTAGATCACAGGCAATCATCCTGTGT
GTCAATCAGGCTCTTCGGGCGGTTTTAGAGACAGTAGTTTTTAAAACCAATTTAAT
TTTGTATTAGATTAATTGGCATTTAAAAAAAAAAAAAAAAAAA

Full length genomic nucleotide sequence of CkAstV isolated after back passage in chicken (“CkAstV-p5-Ckp5”) (SEQ ID NO:3):

(SEQ ID NO: 3)
CCGAAAGCGAGGGTGTGGGCGATGGCCCAGGCCATGGTGGGGGCTTTTTCAAGCC
TTAACAGGCGAGAAGACCGTAAGTCGGCCCAAGTAACAACTGGGCTTGACAAGGT
CTTCTCGTTCCAGGGCGTGCACGAACTTTTTGTAAGAATGCGTGCGCTGTACGGAA
CAACTACAGCATGGAAAGCCCTGATGAATTGTGAAGCTGTGTACATTAAAGATGTT
AAAACGGCTTTTGGCGCGAATGGTTCCCAAATTGGCTTCTTCTTTGCTGAGACACC
CACAACACCGACTTGGTCTCCGGATGCTGGGATTGCTATATTGACTGAAGGTGAAA
AAACTTGTCTAGCAGCACAGCAGGCAAGGGATCTGCGCCTGAAAGCTTCGTTAAGT
ACAAACAGCTCCCTTGTCCACCAGATTATGGAGAGAACAAGAGAGGCCAAGGAGA
AAACGAAACAACTTGAAGAACTTCAACAACGGATTGATAATATGGTGGACACTAA
TAAGGTTCTTTACCAACGCATGGAACAGCGGCACCAAGAAAAATTGGAAGCTCTT
GGTGAGAAAATTAGTAAACTTAGGCATGATAACCATCAGTGGTTCATGACTTGTGA
GAAAAAGGATGAGGAAATAACAAAACTTAAACATCAACTTGAAACCAAGAAGAA
TGGCTATAAGAAAATGGCCTGGGATGCAGTTGCTTGGTTGGTGTTAGCTATCCTTTT
ATTCAGCTTTTTCTCTGTGAGTGAAGGTGCCACACCGAATGGAACCTTACCAATTA
AGGAAGTCAGTTGGAATTTTGATGATGTGGAAAAAACCTGTATGAAACCTGACTTT
GGCTGCTTGGTTATGGATACATGGTTACCACATCCTGTTTTAACATTTGAGGACCTA
ATGTCTAAGTGCTACAATACTCATGGGAATGTCATACCTCGTAGCGCTTTTAGTAG
TGAACAACTCTTGTATGACTGCACAAAAACGGCACACTATTTTAACGATGGGCATG
ACTACATTGAAAATTACCACTGGTGTGAGAAAAGGTTAGCAACTCTGGTGGCAGC
AAACTGTCAAGGAGATAATGGTGTTGATAAGATCTATACGCAAGTTGTCGAGGCTG
TGGCAGCAAGTAGGAAATTCTTTCAGAAAATGGCACTTTACAAGCTTGATGTTTGG
ATACTAGCCATTTTTAGTATTGTGCTAGCGGGAAATAAGGAAAAAATAGTGAAATT
GACCCCTTTCATAGCGTTAGGGTGGTGGTTCAATTTACCTATATTTCTATTGAGTAC
CGCTGTTAACTTCTTTCCTACAATGGCCCTGCCTTTCATTGCGTTTCAAATTCTTCAA
CCAGGGTTCTTAGTTGTAACAGCCTTCACATTGTGGTTGACATTGACGCTTGCTGCA
TTCTTCTGGAACGATGGAATAGCTATTTTGGTTGAGACATCATTTGCTTTGTTTTAT
ACAATCTTGTTCTTTGTCTGGTCAATGGCAATGACAGTTTGTGCTAGCTTGCAATTG
AGCTTAGCATATCAAATACTTTTATTTTGTGTTTCGCTTAGTGTATACTGTGGAACA
AAGTTCGCTTGTAGTCAGGTTGTAATAACAAATCCTGACGGCACAACTGAAAAAGT
CTCCAGAGTAGGAAAAGTTAGGAAAGCAGCCTTCCAGCAGTGTAAGGGTGTGTTG
ACTTTCTTGCAAACACGGGGAATTATTCCATCCACCCCTGTTAAAACTAATAGTGT
AGTTGTAATCACAGGAAAGAATGGTGCAGGCACTGGCTTTAGATTTATGAATTATA
TTGTTACAGCAGGCCATGTCGTCACCGGAAGTGAATGGGCAACAGCTAAATTTGGT
GACGTTTCAGTTAAGATTAAGAAGGAAAAAGAAATTGACATGTGTGAATGCCCTG
ACACAATTGTTCTTTTTAAATTACCAAAAGAACTGCAGGGAGTTAAACCATTGAGA
TTGGCGCAAAATGTAAAATCAAATTACATGACATTACATGGTTTTTGCCCTAACTTT
GTTAACCCTGTTAGCTTTACTGGCTGGTGTACCATTGATGGGCCTTGGTTGAACAAT
GCTTTTAATACAACTTTTGGCAATAGTGGCGCTCCTTATTGTGACCCCGATGGTAAG
CTCATTGGCATTCATTTAGGAACTCAAGGAGTAACTTCACAAGGCTTTGTTATAAG
TGATGTCCTTAGGAAACAGTTTGAAACACATTATCAGTGTTCGTGTAGGAAGGAGG
AAAGTGTCCAACAACCTACACCTGCACCGCCTGCGCAGTTTGATTATGAAGAGTTC
TTGTCTAAGGTTATAACAGGAACAAAAATTTCCCACCAAGCCATATTGAAACAACA
AGAGGAAATGTTAGAACAAATTCAGCTTGTAACAAAGATCCTTAAGGAAAATGGC
TTACTGGCTGAACAGAAGAAAAAAGGGAAAACTAAAAGAACTGCTCGTGGTGCTA
AGGCTACAATGACAAAGAAATATCTTTCTAAGGGGCACTTTATGAAAATGAAAAT
GTTAAGTGAAGAGGAATACCAGAAATTGGTGGATGAGGGCTTCACTGCTGATGAG
ATTAAGGAAGTTGTTAACAACCTTCGGGAGCAAGCTTGGCTTGAATATTGCATTGA
CAATGATATTGATGATGAAGGTGCAGAAGATTGGTATGACCAGATGTTGAATGAT
GAGGCAATTAATGACCAAATTGATAGGGAAATTGAAGCAAAAATGGAAGATGAGG
GGTTCTACCAAGTAAAAGCTCGAGAAACCCTAGCATCACAATGTAGAAACAAAAA
ACGCAAAACTTTTGTTGAACAGGCTCTTTTGCATATTATAGATCTTAAGCCTCATAA
AGTTAGGACAGTTAAGGTTGAGGTTCAGGATGAGAGTGCTAACCAACTTAAAAAG
GTCTTTAAAAAAATGGTCAAAGACGAGGAGGTTGAACAGGGGCAAACGAAGGCCT
TCTTTTCTTCAGGTGATGATATAGAATATTTTGAAAATCGTGACATTGATTGGAAA
AAATTGGAAATGCCAAAATTACAGGATGAGGTTAAATTTGAGACTATCACGCGCA
ATGGCATAACTCAAATTTCTACTGGTGAGGATAATAAGAAAAACATCCTCAAAGA
AAAGGTCACAAATATACCACAACCATTGACACCAGTCCCACCATTTACGGAGAAT
AATCCGGACACTAAGCCTAAAAAGGAGCAAGTTCTTGAACAACGGAAGAGGGTCT
GTAGGTCATGTGGCAGCGACAAACCCCACAATTTCGTTGTGTGCAAGAAAAAAAA
TGAGCTCTTGTTCTGTGTCTGGTGTGGTATAGTTCACTCTGAGAATCAAGGCCACTC
ACGGAAAATCGAATGCCCTAAGTGCAACAAAGCATTTTCTGGGATAGAAGGTCTT
GAACGCCATGCGAGTGAATGCCCTCCAAAAAACTAGTGGAGGGCCCAGATGATTC
TGGGCCCGATTATGAACCTGTTCCCGATTATTTGAAGATTTTTTCTTGGGAAGATGA
CTTGTTACCTCCAATTGGAAAAGAAGCTCTACCTGATAATGTTATACTTCTTGGTCA
TATTCCAGTTGACAAATTAGTTTCTAGAACAAAAAAGGTCACCGATCCACTACTTG
GTGTTATTACGTCCTGGAAACAGGATGAGTATGACAGTACTACATGGACAGTGAA
GGCGTACACGAAGATGTTTGAAAAATTTTTTTATAAAGAACCATCTGACTTTGTGA
ACAATGAGCCAGAATTGACCATCCTTTGTGATAGGGTGGTGCTTGATGAGCATGAT
TATATGGCTAATAGTTCTATGACGCCTATAACAGCAACTGTCAAAAATGTAGACTC
AACACCAGCTTATCCAAAATTTCAGGAGTTTGATACTGAGGAAGAATATCTCACCC
GTTGTGGATGGAGTGAATACCTTGATGTTATTAAGGACAAAGAAACTCTCAACCAT
AGGCCATTATGGTGGTGCTTCCTTAAAAACGAAGTTCTCAAGAAAAAGAAAATTCA
AGAGAATGACATTAGGATGATTTTATGTACAGATCCAGTTTTTACTCGAATTGGAG
CAATGTTTGATCAGGACCAAAATTCAAAAATGAAAAATATGACAGAAACGAGGGC
CGCTCAAGTGGGCTGGACCCCCTTCTTTGGTGGACTTGACCAGCGAATGCGGAGGT
TAGAGAAGATTGAAAACGCCCAGTTTGTAGAAATGGATTGGACCCGCTTCGATGG
CACTATTCCAAAGGCGCTCTTTTGGCGCATCCGGCAGATCAGATTCTTTTTCCTAGC
GCCCCGGTATAAAACCGCAGCTAACAAGGAATTGTTTGATTGGTACACTAAAAATC
TGCTGGAGAAAATAATTCTCTTACCAACTGGAGAAGTGTGTCAGATAAAAAGGGG
TAATCCTTCCGGGCAGTTCTCCACCACAGTTGATAACAATATGTGTAATGTGTGGC
TAACTACCTTTGAGATAGCATGGCTCCACCGCAAACAGAAAGGTAGACTCCCCACT
CCCACTGAGTTGAGGAAAAATCTCAAGTACATTTGTTATGGTGATGACAGGCTCTT
GGCAGTTTCAAAGGATTTTGTGTCCTACGAGCCTGACACTGTTGTTAAGATGTATG
CAGATATCTTTGGGATGTGGGTGAAACCAGAAAATGTGAAAATCAGAGATAGCCT
AGTTGGTCTCTCGTTCTGTGGGATGACAATTATCAAAAATTCGAGCAACCGTTATG
TAGGTGTTCCCAATGTAAATAAAATTCTTTCTACTCTCAGTACTCCTACAAAAAGG
CTTCCAAATATCGAGGCATTGTGGGGGAAATTGATCTCACTGCGGATCCTTTGCGA
GAATGCTGACCCCGACGTCAGGGACTACTTAGATAAGCAAATCCACTGCGTCGAG
GAGTATGCTGCTGCTGAAGAAATACAGTTACCAGAAGTCGGGCCCGACTTCTTTCA
GAAAATCTGGTAGAGGGATGGACCGAAATAGAGCAGCATGGCCGATAAGGCTGGG
CCGCAGAAGAAGAGGGTATCTAGGCGTGGACGTGGCCGCTCTCGTTCAAGGTCAC
GCTCACGCTCTCGATCAAGAAATCGTGTCAAGAAAACAGTCACGATAGTTGAAAC
AAAAAAGACCCCAAGTAAGTCAATTCTGAAAAAGGAGTTGGACAATCACGAGAGG
AAAGACAGAAAAAGGTTCAAAAAATTGGAAAGGAAACTAAATGGACCAAAAATT
CATGATCGCATGGCAGTTACAACTACTCTTGGTGTTCTTACAGGTAATTCTGATAAT
AATTTAGAGAGGAAGATGAGAGCCTTGCTTAATCCACTTCTGCTAAAATCCCAGAA
CACCGGAGCTTCAGCGTCACCACTCTCTCTTAGAGCATCACAATATTCTATGTGGA
AAATACAGCGGTGTGTTGTTAAATTTGTGCCGTTGGTGGGTGCAGCAAATGTTGCT
GGAAGTGTGTCATTTGTATCATTAGACCAGGATGCAACATCATCCCAACCTGAATC
ACCTGACACTATAAAAGCGAAGGTACATGCAGAAGTCGCAATTGGGCAGAGGTTC
AATTGGAATGTGCAATCCAGGTACCTGGTTGGACCCCGTTCTGGTTGGTGGGGTAT
GGATACTGGTGAGTCACCAACTGATACGGTTGGACCAGCTCTCGACTTCTGGAACC
TCTACAGAACAGTAAACACACTCCAGACTGGCTCAACATCACAGACGTACACTGC
GCCATTGTTTTCTGTGGAGGTCTTCGCAGTGTATGTGTTCTCAGGATATGAGCCTAA
ACCTGCTCTTGCAACCATGACAAATTCAACTTTTGAAAGCCAGCAGGGAGTAACTA
TAACGAATGGTTCTAATGGGGAACTTCTGCTTAATGTACCAAAGCAGTCAGGTCTT
GCCGAGCGACTACGTGAAAAAGAAACACCGCAACGAGGCCAAAACCAGTCAGGC
GGAGTTGGGGAGGTTCTATGGGCAGTTGCATCGGGCGCTGTGGAAGGAGCTGCAG
AAGCACTGGGCCCATGGGGTTGGCTACTTCGGGGTGGCTGGTGGGTTATTAAAAAA
CTGTTTGGGCGGAGCGCTGAGAATGCTAATGATGTCTATGTAATGTATTCATCTATT
GAGGATGCTAATAAAGATAGCAGAATTTACCAGACAGTCACAAGCGCAGTACCAG
TCCAGCAAGGCCCACTAGTCCTTACCCAAATTTCATCACCCAATGTCAACCAGGCC
GGTGGAGTAGTACAAGTGGGATCGTCACTTACAACTGACTATCTGCCACTTTCCCA
GGCGCAAGTCCCATTTTTGGAAAATGTCCTTTATACTAACACTGGTCAGCCTTTGAC
GTCCAACAAGAGTCATACAATGAGGATAACTGGATTTCCGGCCTCAAAACTAGTA
ACATCGTCATCATCTCAATGGCTGGGTACTGCTGACAAAAGTGTTCAAGCGACAAA
ATGGCTTATGTCTGACTACACCGATACAGGAGTGATCTTTGGTTTCCCCTACTCTAA
TGATCAACCAGGAGAAACATTGGGCAATATCGGTGTGATACACACATCAAAGTCG
CTTTTGAAAACAGTGACATCACGGCACCAGCGTAGGCTGAGCATGGCACCACTAAT
TTCAACCCCAATACCATCGGCTTCGAGGGGGCCAACACAGATGCGTGGATGTTTCG
ACACCCCCTACTATTGGATAAGGGTTTGTGATAACACATGTTCTAATAAACCCACA
AACGGAGCCGTTACACAGCGGTATAATGCATGGGGTGTCATGGTGGTGAGCGTAG
TTCACAATAAAATCTATGTTCTAGCCGGTTATCCTGATGGCCAAAATAGAGTACCA
CAACAGCAAATGGTATGGGACACGTTTGACTGGGATGCCACATTTACCACTGGCAG
GATCTATTCCTCAACATGGCCAGGCACTGAGGAAGAAGTTGAAGATGAGACTGAT
GCTGAGTCTGATATTTCTAGCCTGTTTGATCCAGTGAATGAAGTGGAGCAGGATTT
TCATTTTCAGTGTAGTCTTAAGACATCAGACTACCTAAAAGAGGAGGCGGATTTTT
GGAAGGCAAAGGCACAACAGTTACTAATGGAAAAAGCAATGGAAAAATCAGCTTC
CAATCCTCCTCTTGTCCGCTTTGAGAAGGGCGGACCTGAGCAGCAAAAACAACCTG
CTAGCAGCCGCGGCCACGCCGAGTAGGAACGAGGGTACAGCTGCACCTTCTCAAT
GGAGTTTTTATGCCGTAATCAGGCTTTTCTCCAAAATTAAGGCACCGGGGCCACGC
CGAGTAGGAACGAGGGTACAGTGCCGGGTTGACCCTACCTTAAAAGGGCGTCCGC
CGGCATGCAATCACCATGTCGGGGTCTGGTTTAGATCACAGGCAATCATCCTGTGT
GTCAATCAGGCTCTTCGGGCGGTTTTAGAGACAGTAGTTTTTAAAACCAATTTAAT
TTTGTATTAGATTAATTGGCATTTA

Amino acid sequence of ORF1a of CkAstV isolated from the intestinal content of RSS affected chickens (“gut”). Also found as Genbank accession number, JF414802 (version JF414802.1, Feb. 15, 2012), which is hereby incorporated by reference in its entirety (SEQ ID NO:4):

(SEQ ID NO: 4)
MAQAMAGAFSSLNRREDRKSAQVTAGLDKVFSFQGVHELFVRMRALSGTTTAWKAL
MNSEAVYIKDVKTAFGANGSQIGFFFAETPTTPTWSPDAGIAILTEGEKTCLAAQQARD
LRLKASLSTNSSLVHQIMEKTREAKEKTKQLEELQHRIDNMVDTNKVLYQRMEQRHQ
EKLEALGEKISKLRYDNHQWFMTCEKKDEEIANLKRQLETKKSGYKKMAWDAVAWF
VLAILLFSFFSVSEGATPNGTLPIKDVSWNFDDVEKTCMKPDFGCLVMDTWLPHPVLT
FEDLMSKCYNTHGNVIPRSAFNSEQLLYDCTKTAHYFNDGHDYIENYHWCEKRLATL
VAANCQGDNGVDKIYTQVVEAVAASRKFFQKMALYKLDVWILAIFSIVLAGNKEKIV
KLTPFIALGWWFNLPIFLLSTAVNFFPTMALPFIAFQILQPGFLVVTAFTLWLTLTLAAFF
WNDGIAILVETSFALFYTILFFVWSMAMTVCVSLQLSLAYQILLFCVSLSVYCGTKFAC
SQVVITNPDGTTEKVSRVGKVRKAAFQQCKGVLTFLQTRGIIPSTPVKTNSVVVITGKN
GAGTGFRFMNYIVTAGHVVTGSEWATAKFGDVSVKIKKEKEIDMCECPDTLVLFKLP
KELQSVKPLRLAQNIKSNYMTLHGFCPNFVNPVSFTGWCTIDGPWLNNAFNTTFGNSG
APYCDPDGKLVGIHLGTQGVTSQGFVICDTLRKQFETHYQCSCRREECDQQPAPAPPV
QFDYEEFLSKVITGTKVSHQAILKQQEEMLEQIQLVTKILQENGLLAEQKKKGKTKRTA
RGAKATMTKKYLSKGHFMKMKMLSEEEYQKLVDEGFTADEIKEVVNNLREQAWLEY
CIDNDIDDEGAEDWYDQMLNDEAINDQIDREIEAKMEDEGFYQSKARETLASQCKDK
KRKTFAEQALLHIIDLKPHKVRTVKVEVQDESANQLKKVFKRMVKDEEVEQGQTKAF
FSSGDDIEYFENRDIDWKKLEMPKLQDEVKFETITRNGITQISTGEDNKKNILKEKVTNI
PQPLTPVPPFTENNPDTKPKKEQVLEQRKRVCRSCGSDKPHNFVVCKKKNELLFCVWC
GIVHSENQGHSRKIECPKCNKAFSGIEGLERHASECPSKN

Amino acid sequence of ORF 1a of CkAstV isolated in cell culture (“CkAstV-p5”) (SEQ ID NO:5):

(SEQ ID NO: 5)
MAQAMAGAFSSLNRREDRKSAQVTTGLDKVFSFQGVHELFVRMRVLYGTSTAWKAL
MNCEAVYIKDVKTAFGANGSQIGFFFAETPTTPTWSPDAGIAILTEGEKTCLAAQQARD
LRLKASLSTNSSLVHQIMERTREAKEKTKQLEELQQRIDNMVDTNKVLYQRMEQRHQ
EKLEALGEKISKLRHDNHQWFMTCEKKDEEITKLKHQLETKKNGYKKMAWDAVAW
LVLAILLFSFFSVSEGATPNGTLPIKEVSWNFDDVEKTCMKPDFGCLVMDTWLPHPVLT
FEDLMSKCYNTHGNVIPRSAFSSEQLLYDCTKTAHYFNDGHDYIENYHWCEKRLATL
VAANCQGDNGVDKIYTQVVEAVAASRKFFQKMALYKLDVWILAIFSIVLAGNKEKIV
KLTPFIALGWWFNLPIFLLSTAVNFFPTMALPFIAFQILQPGFLVVTAFTLWLTLTLAAFF
WNDGIAILVETSFALFYTILFFVWSMAMTVCASLQLSLAYQILLFCVSLSVYCGTKFAC
SQVVITNPDGTTEKVSRVGKVRKAAFQQCKGVLTFLQTRGIIPSTPVKTNSVVVITGKN
GAGTGFRFMNYIVTAGHVVTGSEWATAKFGDVSVKIKKEKEIDMCECPDTIVLFKLPK
ELQGVKPLRLAQNVKSNYMTLHGFCPNFVNPVSFTGWCTIDGPWLNNAFNTTFGNSG
APYCDPDGKLIGIHLGTQGVTSQGFVISDVLRKQFETHYQCSCRKEESVQQPTPAPPAQ
FDYEEFLSKVITGTKISHQAILKQQEEMLEQIQLVTKILKENGLLAEQKKKGKTKRTAR
GAKATMTKKYLSKGHFMKMKMLSEEEYQKLVDEGFTADEIKEVVNNLREQAWLEYC
IDNDIDDEGAEDWYDQMLNDEAINDQIDREIEAKMEDEGFYQVKARETLASQCRNKK
RKTFVEQALLHIIDLKPHKVRTVKVEVQDESANQLKKVFKKMVKDEEVEQGQTKAFF
SSGDDIEYFENRDIDWKKLEMPKLQDEVKFETITRNGITQISTGEDNKKNILKEKVTNIP
QPLTPVPPFTENNPDTKPKKEQVLEQRKRVCRSCGSDKPHNFVVCKKKNELLFCVWC
GIVHSENQGHSRKIECPKCNKAFSGIEGLERHASECPPKN

Amino acid sequence of ORF 1a of CkAstV isolated after back passage in chicken (“CkAstV-p5-Ckp5”) (SEQ ID NO:6):

(SEQ ID NO: 6)
MAQAMVGAFSSLNRREDRKSAQVTTGLDKVFSFQGVHELFVRMRALYGTTTAWKAL
MNCEAVYIKDVKTAFGANGSQIGFFFAETPTTPTWSPDAGIAILTEGEKTCLAAQQARD
LRLKASLSTNSSLVHQIMERTREAKEKTKQLEELQQRIDNMVDTNKVLYQRMEQRHQ
EKLEALGEKISKLRHDNHQWFMTCEKKDEEITKLKHQLETKKNGYKKMAWDAVAW
LVLAILLFSFFSVSEGATPNGTLPIKEVSWNFDDVEKTCMKPDFGCLVMDTWLPHPVLT
FEDLMSKCYNTHGNVIPRSAFSSEQLLYDCTKTAHYFNDGHDYIENYHWCEKRLATL
VAANCQGDNGVDKIYTQVVEAVAASRKFFQKMALYKLDVWILAIFSIVLAGNKEKIV
KLTPFIALGWWFNLPIFLLSTAVNFFPTMALPFIAFQILQPGFLVVTAFTLWLTLTLAAFF
WNDGIAILVETSFALFYTILFFVWSMAMTVCASLQLSLAYQILLFCVSLSVYCGTKFAC
SQVVITNPDGTTEKVSRVGKVRKAAFQQCKGVLTFLQTRGIIPSTPVKTNSVVVITGKN
GAGTGFRFMNYIVTAGHVVTGSEWATAKFGDVSVKIKKEKEIDMCECPDTIVLFKLPK
ELQGVKPLRLAQNVKSNYMTLHGFCPNFVNPVSFTGWCTIDGPWLNNAFNTTFGNSG
APYCDPDGKLIGIHLGTQGVTSQGFVISDVLRKQFETHYQCSCRKEESVQQPTPAPPAQ
FDYEEFLSKVITGTKISHQAILKQQEEMLEQIQLVTKILKENGLLAEQKKKGKTKRTAR
GAKATMTKKYLSKGHFMKMKMLSEEEYQKLVDEGFTADEIKEVVNNLREQAWLEYC
IDNDIDDEGAEDWYDQMLNDEAINDQIDREIEAKMEDEGFYQVKARETLASQCRNKK
RKTFVEQALLHIIDLKPHKVRTVKVEVQDESANQLKKVFKKMVKDEEVEQGQTKAFF
SSGDDIEYFENRDIDWKKLEMPKLQDEVKFETITRNGITQISTGEDNKKNILKEKVTNIP
QPLTPVPPFTENNPDTKPKKEQVLEQRKRVCRSCGSDKPHNFVVCKKKNELLFCVWC
GIVHSENQGHSRKIECPKCNKAFSGIEGLERHASECPPKN

Amino acid sequence of ORF1b of CkAstV isolated from the intestinal content of RSS affected chickens (“gut”). Also found as Genbank accession number, JF414802 (version JF414802.1, Feb. 15, 2012), which is hereby incorporated by reference in its entirety (SEQ ID NO:7):

(SEQ ID NO: 7)
MPFKKLVEGPDDSGPDYEPVPDYLKIFSWEDDLLPPIGKEALPENVILLGHIPVDKLVSR
TKKVTDPLLGVITSWKQDEYDSTTWTVKAYTKMFEKFFYKEPSDFVNNEPELTILCDR
VVLDEHDYMANSSMTPITATIKNVDSTPAYPKFQEFDTEEEYLTRCGWSEYLDVIKNK
ETLNHRPLWWCFLKNEVLKKKKIQENDIRMILCTDPVFTRIGAMFDQDQNSKMKNMT
ETRAAQVGWTPFFGGLDQRMRRLEKIENAQFVEMDWTRFDGTIPKALFWRIRQIRFFF
LAPRYKTAANKELFDWYTKNLLEKIILLPTGEVCQIKRGNPSGQFSTTVDNNMCNVWL
TTFEIAWLHRKQKGRLPTPTELRKNLKYICYGDDRLLAVSKDFVVYEPDTVVKMYAD
VFGMWVKPENVKVRDSLVGLSFCGMTIIKNSNNRYVGVPNVNKILSTLSTPTKRLPNIE
ALWGKLISLRILCENADPDVRDYLDKQIHCVEEYAAAEDIQLPEVGPDFFQKIW

Amino acid sequence of ORF1b of CkAstV isolated in cell culture (CkAstV-p5) (SEQ ID NO:8):

(SEQ ID NO: 8)
MPSKKLVEGPDDSGPDYEPVPDYLKIFSWEDDLLPPIGKEALPDNVILLGHIPVDKLVS
RTKKVTDPLLGVITSWKQDEYDSTTWTVKAYTKMFEKFFYKEPSDFVNNEPELTILCD
RVVLDEHDYMANSSMTPITATVKNVDSTPAYPKFQEFDTEEEYLTRCGWSEYLDVIKD
KETLNHRPLWWCFLKNEVLKKKKIQENDIRMILCTDPVFTRIGAMFDQDQNSKMKNM
TETRAAQVGWTPFFGGLDQRMRRLEKIENAQFVEMDWTRFDGTIPKALFWRIRQIRFF
FLAPRYKTAANKELFDWYTKNLLEKIILLPTGEVCQIKRGNPSGQFSTTVDNNMCNVW
LTTFEIAWLHRKQKGRLPTPTELRKNLKYICYGDDRLLAVSKDFVSYEPDTVVKMYAD
IFGMWVKPENVKIRDSLVGLSFCGMTIIKNSSNRYVGVPNVNKILSTLSTPTKRLPNIEA
LWGKLISLRILCENADPDVRDYLDKQIHCVEEYAAAEEIQLPEVGPDFFQKIW

Amino acid sequence of ORF1b of CkAstV isolated after back passage in chicken (“CkAstV-p5-Ckp5”) (SEQ ID NO:9):

(SEQ ID NO: 9)
MPSKKLVEGPDDSGPDYEPVPDYLKIFSWEDDLLPPIGKEALPDNVILLGHIPVDKLVS
RTKKVTDPLLGVITSWKQDEYDSTTWTVKAYTKMFEKFFYKEPSDFVNNEPELTILCD
RVVLDEHDYMANSSMTPITATVKNVDSTPAYPKFQEFDTEEEYLTRCGWSEYLDVIKD
KETLNHRPLWWCFLKNEVLKKKKIQENDIRMILCTDPVFTRIGAMFDQDQNSKMKNM
TETRAAQVGWTPFFGGLDQRMRRLEKIENAQFVEMDWTRFDGTIPKALFWRIRQIRFF
FLAPRYKTAANKELFDWYTKNLLEKIILLPTGEVCQIKRGNPSGQFSTTVDNNMCNVW
LTTFEIAWLHRKQKGRLPTPTELRKNLKYICYGDDRLLAVSKDFVSYEPDTVVKMYAD
IFGMWVKPENVKIRDSLVGLSFCGMTIIKNSSNRYVGVPNVNKILSTLSTPTKRLPNIEA
LWGKLISLRILCENADPDVRDYLDKQIHCVEEYAAAEEIQLPEVGPDFFQKIW

Amino acid sequence of ORF2 of CkAstV isolated from the intestinal content of RSS affected chickens (“gut”). Also found as Genbank accession number, JF414802 (version JF414802.1, Feb. 15, 2012), which is hereby incorporated by reference in its entirety (SEQ ID NO:10):

(SEQ ID NO: 10)
MADKAGPQKKRASRRGRGRSRSRSRSRSRSRNRVKKTVTIVETKKTPSKSILKKELDN
HERKDRKRFRKLEKKLNGPKIHDRMAVTTTLGVLTGNSDNNLERKMRALLNPLLLKS
QNTGASASPLSLRASQYSMWKIQRCVVKFVPLVGAANVAGSVSFVSLDQDATSSQPES
PDTIKAKVHAEVSIGQRFNWNVQSRYLVGPRSGWWGMDTGESPTDTVGPALDFWNL
YRTVNTLQTGTTSQVYTAPLFSIEVFTVYAFSGYEPKPALATMTNSTFESQQGVTITNG
SNGELLLNVPQRSAFAERLREKETPQRAQNQTGGVGEVLWAVASGAVEGAAEALGP
WGWLLRGGWWVIKKLFGRTGEDANDVYVMYSSIEDANKDSRIYQTVTGSVQIQQGP
LVLTQISSPNVNTSGGVVQVNSTTPNDYLPLSQESYAETPLKKYVLYDSTGNPVDSNM
SHTMRITGYPESKLVTSSSVWLGTTGKSVQSTKWLMSDYTNTGVIFGFPYTSAPPGAT
VGNIGVIHTAKSLIKTIRYRRQNHLPTTPFESSLIPSASKGPSQMLGCFDTPYVWCRVCD
NTCSTKPTDGAVTQRYNAWGLLVVSLAHDKVYVLSGYPNSQTAVPVQQLVWDTFD
WDANFSTGRIYSAVWPGEDGAEQEGSDTDDADSDISSLFDPMNEVEKDFHFQCSLKTS
DYLKEEADFWKAKAQQLLMEKAMEKPSANPPLVRFEKGGPEQQKQPASSRGHAE

Amino acid sequence of ORF2 of CkAstV isolated in cell culture (“CkAstV-p5”) (SEQ ID NO:11):

(SEQ ID NO: 11)
MADKAGPQKKRVSRRGRGRSRSRSRSRSRSRNRVKKTVTIVETKKTPSKSILKKELDN
HERKDRKRFKKLERKLNGPKIHDRMAVTTTLGVLTGNSDNNLERKMRALLNPLLLKS
QNTGASASPLSLRASQYSMWKIQRCVVKFVPLVGAANVAGSVSFVSLDQDATSSQPES
PDTIKAKVHAEVAIGQRFNWNVQSRYLVGPRSGWWGMDTGESPTDTVGPALDFWNL
YRTVNTLQTGSTSQTYTAPLFSVEVFAVYVFSGYEPKPALATMTNSTFESQQGVTITNG
SNGELLLNVPKQSGLAERLREKETPQRGQNQSGGVGEVLWAVASGAVEGAAEALGP
WGWLLRGGWWVIKKLFGRSAENANDVFVMYSSIEDANKDSRIYQTVTSAVPVQQGP
LVLTQISSPNVNQAGGVVQVGSSLTTDYLPLSQAQVPFLENVLYTNTGQPLTSNKSHT
MRITGFPASKLVTSSSSQWLGTADKSVQATKWLMSDYTDTGVIFGFPYSNDQPGETLG
NIGVIHTSKSLLKTVTSRHQRRLSMAPLISTPIPSASRGPTQMRGCFDTPYYWIRVCDNT
CSNKPTNGAVTQRYNAWGVMVVSVVHNKIYVLAGYPDGQNRVPQQQMVWDTFDW
DATFTTGRIYSSTWPGTEEEVEDETDAESDISSLFDPVNEVEQDFHFQCSLKTSDYLKEE
ADFWKAKAQQLLMEKAMEKSASNPPLVRFEKGGPEQQKQPASSRGHAE

Amino acid sequence of ORF2 of CkAstV isolated after back passage in chicken (“CkAstV-p5-Ckp5”) (SEQ ID NO:12):

(SEQ ID NO: 12)
MADKAGPQKKRVSRRGRGRSRSRSRSRSRSRNRVKKTVTIVETKKTPSKSILKKELDN
HERKDRKRFKKLERKLNGPKIHDRMAVTTTLGVLTGNSDNNLERKMRALLNPLLLKS
QNTGASASPLSLRASQYSMWKIQRCVVKFVPLVGAANVAGSVSFVSLDQDATSSQPES
PDTIKAKVHAEVAIGQRFNWNVQSRYLVGPRSGWWGMDTGESPTDTVGPALDFWNL
YRTVNTLQTGSTSQTYTAPLFSVEVFAVYVFSGYEPKPALATMTNSTFESQQGVTITNG
SNGELLLNVPKQSGLAERLREKETPQRGQNQSGGVGEVLWAVASGAVEGAAEALGP
WGWLLRGGWWVIKKLFGRSAENANDVYVMYSSIEDANKDSRIYQTVTSAVPVQQGP
LVLTQISSPNVNQAGGVVQVGSSLTTDYLPLSQAQVPFLENVLYTNTGQPLTSNKSHT
MRITGFPASKLVTSSSSQWLGTADKSVQATKWLMSDYTDTGVIFGFPYSNDQPGETLG
NIGVIHTSKSLLKTVTSRHQRRLSMAPLISTPIPSASRGPTQMRGCFDTPYYWIRVCDNT
CSNKPTNGAVTQRYNAWGVMVVSVVHNKIYVLAGYPDGQNRVPQQQMVWDTFDW
DATFTTGRIYSSTWPGTEEEVEDETDAESDISSLFDPVNEVEQDFHFQCSLKTSDYLKEE
ADFWKAKAQQLLMEKAMEKSASNPPLVRFEKGGPEQQKQPASSRGHAE

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

### Sequence Listing Free Text

SEQ ID NO:1 Full length genomic nucleotide sequence of a chicken astrovirus (CkAstV) isolated from the intestinal content of RSS affected chickens (“gut”).
SEQ ID NO:2 Full length genomic nucleotide sequence of CkAstV isolated in cell culture (“CkAstV-p5”).
SEQ ID NO:3 Full length genomic nucleotide sequence of CkAstV isolated after back passage in chicken (“CkAstV-p5-Ckp5”).
SEQ ID NO:4 Amino acid sequence of ORF1a of CkAstV isolated from the intestinal content of RSS affected chickens (“gut”).
SEQ ID NO:5 Amino acid sequence of ORF1a of CkAstV-p5 isolated in cell culture.
SEQ ID NO:6 Amino acid sequence of ORF1a of CkAstV-p5-Ckp5 isolated after back passage in chicken.
SEQ ID NO:7 Amino acid sequence of ORF1b of CkAstV isolated from the intestinal content of RSS affected chickens (“gut”).
SEQ ID NO:8 Amino acid sequence of ORF1b of CkAstV-p5 isolated in cell culture.
SEQ ID NO:9 Amino acid sequence of ORF1b of CkAstV-p5-Ckp5 isolated after back passage in chicken.
SEQ ID NO:10 Amino acid sequence of ORF2 of CkAstV isolated from the intestinal content of RSS affected chickens (“gut”).
SEQ ID NO:11 Amino acid sequence of ORF2 of CkAstV-5p isolated in cell culture.
SEQ ID NO:12 Amino acid sequence of ORF2 of CkAstV-p5-Ckp5 isolated after back passage in chicken.
SEQ ID NO:13-22 Synthetic oligonucleotide primers
SEQ ID NO:23-28 Amino acid sequences of chicken astrovirus
SEQ ID NO:29-38 Conserved nucleotides in the noncoding regions of bird astroviruses

What is claimed is:

1. An isolated chicken astrovirus, the chicken astrovirus comprising a full length genomic sequence with at least 85% sequence identity to the genomic sequence of the cell culture chicken astrovirus CkAstV-p5 (SEQ ID NO:2) and/or at least 85% sequence identity to the genomic sequence the chicken astrovirus after back passage in chicken CkAstV-p5-Ckp5 (SEQ ID NO:3), and attenuations and derivatives thereof.

2. The isolated chicken astrovirus of claim 1, wherein the chicken astrovirus comprises an open reading frame 1a (ORF1a) with an amino acid sequence with at least about 90% sequence identity to SEQ ID NO:5 and/or SEQ ID NO:6.

3. The isolated chicken astrovirus of claim 1, wherein the chicken astrovirus comprises an open reading frame 1b (ORF1b) with an amino acid sequence with at least about 90% sequence identity to SEQ ID NO:8 and/or SEQ ID NO:9.

4. The isolated chicken astrovirus of claim 1, wherein the chicken astrovirus comprises an open reading frame 2 (ORF2) with an amino acid sequence with at least about 90% sequence identity to SEQ ID NO:11 and/or SEQ ID NO:12.

5. The isolated chicken astrovirus of claim 1, wherein the chicken astrovirus comprises at least one nucleotide substitution modification relative to SEQ ID NO:1.

6. A virus of claim 1, wherein the virus is attenuated, inactivated, or killed.

7. A cell culture supernatant, the cell culture supernatant comprising a chicken astrovirus of claim 1.

8. An isolated chicken cell line, the cells infected with a chicken astrovirus of claim 1

9. The chicken cell line of claim 8, wherein the cell line is LMH.

10. An isolated polynucleotide sequence having at least about 90% sequence identity to a chicken astrovirus (CkAst) genomic sequence comprising SEQ ID NO:2 or SEQ ID NO:3, a truncation, or fragment thereof.

11. An isolated polypeptide having an amino acid sequence with at least about 90% sequence identity to an amino acid sequence selected from SEQ ID NO:4, SEQ ID NO:5, SEQ ID NO:6, SEQ ID NO:7, SEQ ID NO:8, SEQ ID NO:9, SEQ ID NO:10, SEQ ID NO:11, or SEQ ID NO:12.

12. An immunological composition for raising antibodies in poultry, the composition comprising a virus of claim 1.

13. A composition of claim 13, further comprising an adjuvant.

14. A composition of claim 12, further comprising an antigenic determinant from one or more additional pathogens infectious to poultry.

15. A diagnostic kit comprising a virus of claim 1.

16. A method of detecting exposure to runting-stunting syndrome (RSS) in a bird, the method comprising determining that an antisera sample obtained from the bird specifically binds to a virus of claim 1.

17. A method of producing an anti-RSS immune response in poultry, the method comprising administering an isolated virus of claim 1.

18. The method of claim 17, wherein immunity comprises humoral and/or cellular immunity.

19. The method of claim 17, wherein immunity comprises mucosal immunity.

20. A method of preventing RSS in poultry, the method comprising administering a composition of claim 12.

21. The method of claim 20, wherein administration comprises injection, spraying, oral administration, or respiratory administration.

22. The method of claim 20, wherein administration induces mucosal immunity.

23. The method of 23, wherein administration comprises in ovo administration.


SEQUENCE LISTING (52 sequences)

SEQ ID NO: 1 - DNA, 7539 bp, chicken astrovirus
ccgaaagcgagggtgtgggcgatggcccaggccatggcgggggctttttcaagccttaacaggcgagaagaccgtaagtcggcccaagtaacagctgggcttgacaaggtcttctcgttccagggcgtgcacgaactttttgtaagaatgcgtgcgctgtccggaacaactacagcatggaaagccctgatgaatagtgaagctgtgtatattaaagatgtaaaaacggcttttggcgcgaatggttcccaaattggcttcttctttgctgagacacccacaacaccaacttggtctccagatgctgggattgctatattgactgaaggtgaaaaaacttgtcttgcagcacagcaggcaagggatctgcgcctgaaagcttcactaagcacaaacagctctctggtccaccagattatggagaagacaagagaggccaaggagaaaacgaaacaactcgaagaactccagcatcgtattgacaatatggtggatactaataaagtcttgtatcaacgcatggaacagcggcatcaggagaagttggaagcccttggtgagaagatcagtaaacttagatatgataaccatcagtggttcatgacttgtgagaaaaaggatgaggaaatagcaaacctcaagcgtcaacttgaaactaaaaagagtggctataagaaaatggcctgggacgcagttgcttggtttgtgctagctatcctcttattcagttttttctctgtgagtgaaggtgccacacctaatggaactttaccaattaaggatgttagttggaactttgatgacgtggaaaaaacttgcatgaaacctgattttggctgtttggttatggacacctggctaccacatcccgtcctaacgtttgaggacttaatgtctaagtgctacaatactcatgggaatgtcatacctcgtagtgcttttaatagtgaacaactcttgtatgactgcacaaaaacggcacactattttaacgatgggcatgactacattgaaaattaccactggtgtgagaaaaggttagcaactctggtggcagcaaactgtcaaggagataatggtgttgataagatctatacgcaagttgtcgaggctgtggcagcaagtaggaaattctttcagaaaatggcactctataagcttgatgtttggatactagctatttttagtattgtgttagcgggaaataaggaaaaaatagtaaaattaaccccttttatagcgttaggctggtggtttaatttacctatattcctactgagtaccgctgttaacttctttcctacaatggccttgccttttattgcgttccagattctccaaccagggtttctagttgtaacagcctttacattgtggttgacactaacacttgctgctttcttttggaatgatggaatagccatcttagttgagacgtcatttgccttgttctacacgatcttgttttttgtttggtcgatggcaatgacagtctgtgttagtttacagttgagtttagcgtaccaaatactcttgttctgtgtttcgcttagtgtgtattgtggaacaaagtttgcgtgtagtcaggttgttataacaaatcctgatggaacaacagaaaaagtttcaagagtaggtaaggttaggaaagcagcctttcaacagtgtaagggtgtattgaccttcttgcaaacacgggggattattccatccacacctgttaaaactaatagtgtagtcgttattacaggaaagaatggtgcaggtactggatttaggtttatgaattatattgtcacagcaggccatgttgtcaccggtagtgaatgggcaacagctaaattcggtgatgtttcagttaaaattaagaaggaaaaagaaattgacatgtgtgagtgccctgacacacttgtcctttttaaattaccaaaggaattgcagagtgttaaaccattaagattagcccaaaatataaagtcaaattatatgacattgcacgggttttgtccaaattttgtcaaccctgttagttttacaggctggtgtactattgatggaccttggctaaacaatgcttttaatacaacttttggtaatagtggtgctccatactgtgatcccgatgggaagcttgttggtattcacctaggaacccaaggagtaacttcacaaggttttgttatatgtgatactcttagaaagcaatttgaaacacactatcagtgttcgtgcaggagagaagagtgtgatcaacaacctgcaccggcaccacctgtgcagttcgactatgaagaatttttgtctaaagttataacaggaacaaaggtttcccatcaggccatactgaaacaacaagaagaaatgttagagcaaattcagcttgtaacaaagattcttcaggaaaatggcttgttagctgaacagaagaagaaaggaaaaacaaaaagaactgctcgtggtgctaaggctacaatgacaaagaaatacctttccaaaggacacttcatgaaaatgaaaatgttaagtgaagaggagtaccaaaaattggtggatgaaggcttcactgccgatgagattaaagaagttgtcaacaaccttcgggagcaagcatggcttgaatattgtattgacaatgatattgatgatgagggagcagaagattggtatgatcagatgttaaatgatgaggcaattaatgaccaaattgatagggaaattgaagcaaaaatggaagatgaaggcttttatcaatcaaaagccagagaaactttggcatcacagtgcaaagataaaaaacgcaaaacttttgctgaacaggcccttctgcatattatagatcttaaacctcataaggttaggacagttaaggttgaggttcaggacgagagtgccaaccaactcaaaaaggtctttaagagaatggtcaaagacgaggaagttgaacaggggcaaacgaaggccttcttttcttcaggtgatgatatagaatattttgaaaatcgtgatattgattggaaaaaattggaaatgccaaaattacaggatgaggttaaatttgagaccatcacgcgcaatggcataactcaaatttctactggtgaggataataagaaaaacatcctcaaagaaaaggtcacaaatataccacaaccgttgacaccagttccaccgtttacggagaataatccggacactaagcctaaaaaggagcaggttcttgaacaacggaagagggtttgtaggtcatgtggtagcgacaaaccccacaacttcgttgtgtgtaagaaaaagaatgagctcttgttctgtgtctggtgtggtatagtgcactctgagaaccaaggccactcacggaaaatcgaatgccctaaatgcaacaaggcattctctgggatagagggtcttgaacgccatgcgagtgaatgcccttcaaaaaactagtggagggcccagatgattctgggcccgattatgaacctgtccccgactatttgaagattttttcttgggaagatgacttgctacctccaattggaaaagaagctctacctgaaaatgttatacttcttggccatattccggttgataaattggtttctagaacaaagaaggtaactgatccactacttggagttatcacttcttggaaacaggatgaatatgatagtaccacatggactgttaaggcgtacactaaaatgtttgagaaattcttctataaagaaccatcagattttgtgaataatgagccagagctcaccatcctatgtgatagggtggtgcttgatgaacatgattatatggcaaatagttctatgacgcctattacagcgactataaaaaatgtggactcaacacctgcttatccaaaattccaggagtttgataccgaagaagaatatcttacccgttgtggatggagtgaatacctcgatgtcattaagaacaaagaaactctcaaccataggccattatggtggtgcttccttaaaaacgaagttctcaagaaaaagaaaattcaagagaatgatattaggatgattttatgtacagatccagtcttcactcgaattggagcaatgtttgatcaggaccaaaattcaaaaatgaaaaatatgacagaaacgagggccgcacaagtgggctggacccccttctttggtggacttgaccagcgaatgcggaggttggagaagattgaaaacgcccagtttgtagaaatggattggacccgctttgatggcactattccaaaggcgcttttttggcgcatccggcagatcagattctttttcctagcgccccggtataaaaccgcggctaacaaggaattgtttgattggtacactaaaaatctgctggagaaaataattctcttaccaactggagaagtgtgtcagataaaaaggggtaatccttctgggcagttctccaccacagttgataacaatatgtgtaatgtgtggttaactacctttgaaatagcatggctccaccgcaaacagaagggcagactccccactcccactgagttgaggaaaaatcttaagtacatttgctatggtgatgacaggctcttggcagtttcaaaggattttgtggtctatgagcctgacactgttgttaaaatgtatgcagatgtatttgggatgtgggtgaaaccagaaaatgtaaaagttagagatagcctagttggtctctcgttctgtgggatgacaattatcaaaaattcgaacaaccgttatgtaggggttcccaatgtaaataaaattctttctactctcagtactcctacaaaaaggcttccaaatatcgaggccttgtgggggaaattgatctcactgcggatcctttgtgagaatgctgatcccgacgtcagggactacttagataaacagatccactgcgtcgaggaatatgctgctgctgaagatatacagttaccagaagtcgggcccgacttctttcagaaaatctggtagagggatggaccgaaatagagcagcatggccgataaggctgggccgcagaagaagagggcatctaggcgcgggcgtggccgctctcgctcaaggtcacgctcacgctctcgatcaagaaatcgtgtcaagaaaacagtcacgatcgttgaaacaaaaaagaccccaagtaagtcaatcctgaaaaaggagttggataatcatgagaggaaagatagaaaaaggttcagaaaattggaaaagaaattaaatggaccaaaaattcatgatcgcatggcggttacaaccactcttggtgttctcactggcaattctgacaataatttggaaaggaagatgagggcattgctcaaccccttactcttaaaatcccagaacactggagcatcggcatcaccactctctcttagagcctcacagtattcaatgtggaaaatacagcggtgtgttgttaaatttgtaccgctggtgggagcagcaaatgttgctggaagtgtgtcatttgtgtcgttggaccaagatgcaacatcctctcaacctgaatcacctgatactataaaggcgaaagtgcatgccgaggtgtccattggccagaggttcaactggaatgtgcaatctagataccttgttggaccgcggtctggctggtggggtatggacaccggagagtcaccaactgacacagttggtccagcacttgatttttggaatttatataggacagtaaatactcttcagactggaacaacatcacaagtctacactgctccattattctctatagaagtgttcaccgtctatgcattctcgggatacgagcctaaacctgcccttgctacaatgacaaattctacttttgaaagtcagcagggggtaaccataacaaatggttccaatggtgaacttcttcttaacgttccacaaaggtcggcatttgcagaaaggctccgcgaaaaagagacaccacagcgtgcgcaaaatcaaaccggcggggttggagaggtgctgtgggctgtagcatctggtgcagtggaaggggctgcagaagcgttgggcccgtggggctggctactaagaggtggctggtgggtcattaaaaaattatttggtagaaccggagaagatgcaaatgatgtgtacgtgatgtattcatcaattgaagatgcaaacaaagacagtagaatatatcaaactgtcactggatcggtgcaaatacaacaaggcccactcgttctaacacaaatctcatcgccgaatgtgaatacatccggaggggttgttcaggtaaattcaaccactccaaatgactacttgcccctctctcaagaaagttatgcagagacaccattgaaaaaatatgtactttatgacagcaccgggaaccccgttgatagcaatatgagccacaccatgaggataacagggtatcctgagtcaaaactagtgacatcaagctcagtctggctcggtacaactggtaagagcgtacaatcaactaaatggctgatgtccgattacacaaatacaggggtcatatttggttttccttatacgagcgcaccaccgggagcaactgtcggcaacattggtgtcattcatactgcaaaatcattaattaagacaatcagatacagaagacaaaaccatcttccaacaacaccttttgaatcttccctgataccgtcagcgtcaaaaggacccagtcaaatgctaggatgttttgacaccccatacgtatggtgtagagtttgtgataatacatgctccaccaagcctactgatggtgcagttacacagaggtacaatgcatggggcctgctggtggttagccttgcccatgataaggtgtatgtactatcaggctatccaaattcacaaacagcagtaccagtgcaacaattggtttgggatacttttgactgggatgcaaatttttccactggcagaatttatagcgcagtatggccaggtgaagatggtgctgaacaggaaggttcagacactgatgatgctgactctgacatttccagtctttttgacccaatgaatgaggtggaaaaggacttccatttccagtgtagtctcaaaacttctgactacttaaaagaggaggctgacttttggaaagcaaaggcgcaacagctactcatggagaaggcaatggaaaaaccaagtgctaatcctcctcttgtccgcttcgagaagggtggacctgagcagcaaaaacaacctgctagcagccgcggccacgccgagtaggatcgagggtacagctgcacctcctcaatggagtttttatgccgtaatcaggcttttctccattcaaaaattaaggcaccggggccacgccgagtaggatcgagggtacagtgccgggttgacctcaccttaaagaggcgtccgccggtatggaaatcaccatgctggagtctggttaagatcacagacaatcactctgtgtgtaaatcaggcttgtcgggcggttttggaaacgtaggtttttaaaaccaatttgattttgaattaaattaattggcatttaaaaaaaaaaaaaaaaaaa

SEQ ID NO: 2 - DNA, 7518 bp, chicken astrovirus
ccgaaagcgagggtgtgggcgatggcccaggccatggcgggggctttttcaagccttaataggcgagaagaccgtaagtcagcccaagtaacaactgggcttgacaaggtcttctcgttccagggcgtgcacgaactcttcgtgagaatgcgtgtgctgtatggaacatctacagcatggaaagccctgatgaattgtgaagctgtgtacattaaagatgttaaaacggcttttggcgcgaatggttcccaaattggcttcttctttgctgagacacccacaacaccgacttggtctccggatgctgggattgctatattgactgaaggtgaaaaaacttgtctagcagcacagcaggcaagggatctgcgcctgaaagcttcgttaagtacaaacagctcccttgtccaccagattatggagagaacaagagaggccaaggagaaaacgaaacaacttgaagaacttcaacaacggattgataatatggtggacactaataaggttctttaccaacgcatggaacagcggcaccaagaaaaattggaagctcttggtgagaaaattagtaaacttaggcatgataaccatcagtggttcatgacttgtgagaaaaaggatgaggaaataacaaaacttaaacatcaacttgaaaccaagaagaatggctataagaaaatggcctgggatgcagttgcttggttggtgttagctatccttttattcagctttttctctgtgagtgaaggtgccacaccgaatggaaccttaccaattaaggaagtcagttggaattttgatgatgtggaaaaaacctgtatgaaacctgactttggctgcttggttatggatacatggttaccacatcctgttttaacatttgaggacctaatgtctaagtgctacaatactcatgggaatgtcatacctcgtagcgcttttagtagtgaacaactcttgtatgactgcacaaaaacggcacactattttaacgatgggcatgactacattgaaaattaccactggtgtgagaaaaggttagcaactctggtggcagcaaactgtcaaggagataatggtgttgataagatctatacgcaagttgtcgaggctgtggcagcaagtaggaaattctttcagaaaatggcactttacaagcttgatgtttggatactagccatttttagtattgtgctagcgggaaataaggaaaaaatagtgaaattgacccctttcatagcgttagggtggtggttcaatttacctatatttctattgagtaccgctgttaacttctttcctacaatggccctgcctttcattgcgtttcaaattcttcaaccagggttcttagttgtaacagccttcacattgtggttgacattgacgcttgctgcattcttctggaacgatggaatagctattttggttgagacatcatttgctttgttttatacaatcttgttctttgtctggtcaatggcaatgacagtttgtgctagcttgcaattgagcttagcatatcaaatacttttattttgtgtttcgcttagtgtatactgtggaacaaagttcgcttgtagtcaggttgtaataacaaatcctgacggcacaactgaaaaagtctccagagtaggaaaagttaggaaagcagccttccagcagtgtaagggtgtgttgactttcttgcaaacacggggaattattccatccacccctgttaaaactaatagtgtagttgtaatcacaggaaagaatggtgcaggcactggctttagatttatgaattatattgttacagcaggccatgtcgtcaccggaagtgaatgggcaacagctaaatttggtgacgtttcagttaagattaagaaggaaaaagaaattgacatgtgtgaatgccctgacacaattgttctttttaaattaccaaaagaactgcagggagttaaaccattgagattggcgcaaaatgtaaaatcaaattacatgacattacatggtttttgccctaactttgttaaccctgttagctttactggctggtgtaccattgatgggccttggttgaacaatgcttttaatacaacttttggcaatagtggcgctccttattgtgaccccgatggtaagctcattggcattcatttaggaactcaaggagtaacttcacaaggctttgttataagtgatgtccttaggaaacagtttgaaacacattatcagtgttcgtgtaggaaggaggaaagtgtccaacaacctacacctgcaccgcctgcgcagtttgattatgaagagttcttgtctaaggttataacaggaacaaaaatttcccaccaagccatattgaaacaacaagaggaaatgttagaacaaattcagcttgtaacaaagatccttaaggaaaatggcttactggctgaacagaagaaaaaagggaaaactaaaagaactgctcgtggtgctaaggctacaatgacaaagaaatatctttctaaggggcactttatgaaaatgaaaatgttaagtgaagaggaataccagaaattggtggatgagggcttcactgctgatgagattaaggaagttgttaacaaccttcgggagcaagcttggcttgaatattgcattgacaatgatattgatgatgaaggtgcagaagattggtatgaccagatgttgaatgatgaggcaattaatgaccaaattgatagggaaattgaagcaaaaatggaagatgaggggttctaccaagtaaaagctcgagaaaccctagcatcacaatgtagaaacaaaaaacgcaaaacttttgttgaacaggctcttttgcatattatagatcttaagcctcataaagttaggacagttaaggttgaggttcaggatgagagtgctaaccaacttaaaaaggtctttaaaaaaatggtcaaagacgaggaggttgaacaggggcaaacgaaggccttcttttcttcaggtgatgatatagaatattttgaaaatcgtgacattgattggaaaaaattggaaatgccaaaattacaggatgaggttaaatttgagactatcacgcgcaatggcataactcaaatttctactggtgaggataataagaaaaacatcctcaaagaaaaggtcacaaatataccacaaccattgacaccagtcccaccatttacggagaataatccggacactaagcctaaaaaggagcaagttcttgaacaacggaagagggtctgtaggtcatgtggcagcgacaaaccccacaatttcgttgtgtgcaagaaaaaaaatgagctcttgttctgtgtctggtgtggtatagttcactctgagaatcaaggccactcacggaaaatcgaatgccctaagtgcaacaaagcattttctgggatagaaggtcttgaacgccatgcgagtgaatgccctccaaaaaactagtggagggcccagatgattctgggcccgattatgaacctgttcccgattatttgaagattttttcttgggaagatgacttgttacctccaattggaaaagaagctctacctgataatgttatacttcttggtcatattccagttgacaaattagtttctagaacaaaaaaggtcaccgatccactacttggtgttattacgtcctggaaacaggatgagtatgacagtactacatggacagtgaaggcgtacacgaagatgtttgaaaaatttttttataaagaaccatctgactttgtgaacaatgagccagaattgaccatcctttgtgatagggtggtgcttgatgagcatgattatatggctaatagttctatgacgcctataacagcaactgtcaaaaatgtagactcaacaccagcttatccaaaatttcaggagtttgatactgaggaagaatatctcacccgttgtggatggagtgaataccttgatgttattaaggacaaagaaactctcaaccataggccattatggtggtgcttccttaaaaacgaagttctcaagaaaaagaaaattcaagagaatgacattaggatgattttatgtacagatccagtttttactcgaattggagcaatgtttgatcaggaccaaaattcaaaaatgaaaaatatgacagaaacgagggccgctcaagtgggctggacccccttctttggtggacttgaccagcgaatgcggaggttagagaagattgaaaacgcccagtttgtagaaatggattggacccgcttcgatggcactattccaaaggcgctcttttggcgcatccggcagatcagattctttttcctagcgccccggtataaaaccgcggctaacaaggaattgtttgattggtacactaaaaatctgctggagaaaataattctcttaccaactggagaagtgtgtcagataaaaaggggtaatccttccgggcagttctccaccacagttgataacaatatgtgtaatgtgtggctaactacctttgagatagcatggctccaccgcaaacagaaaggtagactccccactcccactgagttgaggaaaaatctcaagtacatttgttatggtgatgacaggctcttggcagtttcaaaggattttgtgtcctacgagcctgacactgttgttaagatgtatgcagatatctttgggatgtgggtgaaaccagaaaatgtgaaaatcagagatagcctagttggtctctcgttctgtgggatgacaattatcaaaaattcgagcaaccgttatgtaggtgttcccaatgtaaataaaattctttctactctcagtactcctacaaaaaggcttccaaatatcgaggcattgtgggggaaattgatctcactgcggatcctttgcgagaatgctgaccccgacgtcagggactacttagataagcaaatccactgcgtcgaggagtatgctgctgctgaagaaatacagttaccagaagtcgggcccgacttctttcagaaaatctggtagagggatggaccgaaatagagcagcatggccgataaggctgggccgcagaagaagagggtatctaggcgtggacgtggccgctctcgttcaaggtcacgctcacgctctcgatcaagaaatcgtgtcaagaaaacagtcacgatagttgaaacaaaaaagaccccaagtaagtcaattctgaaaaaggagttggacaatcacgagaggaaagacagaaaaaggttcaaaaaattggaaaggaaactaaatggaccaaaaattcatgatcgcatggcagttacaactactcttggtgttcttacaggtaattctgataataatttagagaggaagatgagagccttgcttaatccacttctgctaaaatcccagaacaccggagcttcagcgtcaccactctctcttagagcatcacaatattctatgtggaaaatacagcggtgtgttgttaaatttgtgccgttggtgggtgcagcaaatgttgctggaagtgtgtcatttgtatcattagaccaggatgcaacatcatcccaacctgaatcacctgacactataaaagcgaaggtacatgcagaagtcgcaattgggcagaggttcaattggaatgtgcaatccaggtacctggttggaccccgttctggttggtggggtatggatactggtgagtcaccaactgatacggttggaccagctctcgacttctggaacctctacagaacagtaaacacactccagactggctcaacatcacagacgtacactgcgccattgttttctgtggaggtcttcgcagtgtatgtgttctcaggatatgagcctaaacctgctcttgcaaccatgacaaattcaacttttgaaagccagcagggagtaactataacgaatggttctaatggggaacttctgcttaatgtaccaaagcagtcaggtcttgccgagcgactacgtgaaaaagaaacaccgcaacgaggccaaaaccagtcaggcggagttggggaggttctatgggcagttgcatcgggcgctgtggaaggagctgcagaagcactgggcccatggggttggctacttcggggtggctggtgggttattaaaaaactgtttgggcggagcgctgagaatgctaatgatgtctttgtaatgtattcatctattgaggatgctaataaagatagcagaatttaccagacagtcacaagcgcagtaccagtccagcaaggcccactagtccttacccaaatttcatcacccaatgtcaaccaggccggtggagtagtacaagtgggatcgtcacttacaactgactatctgccactttcccaggcgcaagtcccatttttggaaaatgtcctttatactaacactggtcagcctttgacgtccaacaagagtcatacaatgaggataactggatttccggcctcaaaactagtaacatcgtcatcatctcaatggctgggtactgctgacaaaagtgttcaagcgacaaaatggcttatgtctgactacaccgatacaggagtgatctttggtttcccctactctaatgatcaaccaggagaaacattgggcaatatcggtgtgatacacacatcaaagtcgcttttgaaaacagtgacatcacggcaccagcgtaggctgagcatggcaccactaatttcaaccccaataccatcggcttcgagggggccaacacagatgcgtggatgtttcgacaccccctactattggataagggtttgtgataacacatgttctaataaacccacaaacggagccgttacacagcggtataatgcatggggtgtcatggtggtgagcgtagttcacaataaaatctatgttctagccggttatcctgatggccaaaatagagtaccacaacagcaaatggtatgggacacgtttgactgggatgccacatttaccactggcaggatctattcctcaacatggccaggcactgaggaagaagttgaagatgagactgatgctgagtctgatatttctagcctgtttgatccagtgaatgaagtggagcaggattttcattttcagtgtagtcttaagacatcagactacctaaaagaggaggcggatttttggaaggcaaaggcacaacagttactaatggaaaaagcaatggaaaaatcagcttccaatcctcctcttgtccgctttgagaagggcggacctgagcagcaaaaacaacctgctagcagccgcggccacgccgagtaggaacgagggtacagctgcaccttctcaatggaggttttatgccgtaatcaggcttttctccaaaattaaggcaccggggccacgccgagtaggaacgagggtacagtgccgggttgaccctaccttaaaagggcgtccgccggcatgcaatcaccatgtcggggtctggtttagatcacaggcaatcatcctgtgtgtcaatcaggctcttcgggcggttttagagacagtagtttttaaaaccaatttaattttgtattagattaattggcatttaaaaaaaaaaaaaaaaaaa

SEQ ID NO: 3 - DNA, 7500 bp, chicken astrovirus
ccgaaagcgagggtgtgggcgatggcccaggccatggtgggggctttttcaagccttaacaggcgagaagaccgtaagtcggcccaagtaacaactgggcttgacaaggtcttctcgttccagggcgtgcacgaactttttgtaagaatgcgtgcgctgtacggaacaactacagcatggaaagccctgatgaattgtgaagctgtgtacattaaagatgttaaaacggcttttggcgcgaatggttcccaaattggcttcttctttgctgagacacccacaacaccgacttggtctccggatgctgggattgctatattgactgaaggtgaaaaaacttgtctagcagcacagcaggcaagggatctgcgcctgaaagcttcgttaagtacaaacagctcccttgtccaccagattatggagagaacaagagaggccaaggagaaaacgaaacaacttgaagaacttcaacaacggattgataatatggtggacactaataaggttctttaccaacgcatggaacagcggcaccaagaaaaattggaagctcttggtgagaaaattagtaaacttaggcatgataaccatcagtggttcatgacttgtgagaaaaaggatgaggaaataacaaaacttaaacatcaacttgaaaccaagaagaatggctataagaaaatggcctgggatgcagttgcttggttggtgttagctatccttttattcagctttttctctgtgagtgaaggtgccacaccgaatggaaccttaccaattaaggaagtcagttggaattttgatgatgtggaaaaaacctgtatgaaacctgactttggctgcttggttatggatacatggttaccacatcctgttttaacatttgaggacctaatgtctaagtgctacaatactcatgggaatgtcatacctcgtagcgcttttagtagtgaacaactcttgtatgactgcacaaaaacggcacactattttaacgatgggcatgactacattgaaaattaccactggtgtgagaaaaggttagcaactctggtggcagcaaactgtcaaggagataatggtgttgataagatctatacgcaagttgtcgaggctgtggcagcaagtaggaaattctttcagaaaatggcactttacaagcttgatgtttggatactagccatttttagtattgtgctagcgggaaataaggaaaaaatagtgaaattgacccctttcatagcgttagggtggtggttcaatttacctatatttctattgagtaccgctgttaacttctttcctacaatggccctgcctttcattgcgtttcaaattcttcaaccagggttcttagttgtaacagccttcacattgtggttgacattgacgcttgctgcattcttctggaacgatggaatagctattttggttgagacatcatttgctttgttttatacaatcttgttctttgtctggtcaatggcaatgacagtttgtgctagcttgcaattgagcttagcatatcaaatacttttattttgtgtttcgcttagtgtatactgtggaacaaagttcgcttgtagtcaggttgtaataacaaatcctgacggcacaactgaaaaagtctccagagtaggaaaagttaggaaagcagccttccagcagtgtaagggtgtgttgactttcttgcaaacacggggaattattccatccacccctgttaaaactaatagtgtagttgtaatcacaggaaagaatggtgcaggcactggctttagatttatgaattatattgttacagcaggccatgtcgtcaccggaagtgaatgggcaacagctaaatttggtgacgtttcagttaagattaagaaggaaaaagaaattgacatgtgtgaatgccctgacacaattgttctttttaaattaccaaaagaactgcagggagttaaaccattgagattggcgcaaaatgtaaaatcaaattacatgacattacatggtttttgccctaactttgttaaccctgttagctttactggctggtgtaccattgatgggccttggttgaacaatgcttttaatacaacttttggcaatagtggcgctccttattgtgaccccgatggtaagctcattggcattcatttaggaactcaaggagtaacttcacaaggctttgttataagtgatgtccttaggaaacagtttgaaacacattatcagtgttcgtgtaggaaggaggaaagtgtccaacaacctacacctgcaccgcctgcgcagtttgattatgaagagttcttgtctaaggttataacaggaacaaaaatttcccaccaagccatattgaaacaacaagaggaaatgttagaacaaattcagcttgtaacaaagatccttaaggaaaatggcttactggctgaacagaagaaaaaagggaaaactaaaagaactgctcgtggtgctaaggctacaatgacaaagaaatatctttctaaggggcactttatgaaaatgaaaatgttaagtgaagaggaataccagaaattggtggatgagggcttcactgctgatgagattaaggaagttgttaacaaccttcgggagcaagcttggcttgaatattgcattgacaatgatattgatgatgaaggtgcagaagattggtatgaccagatgttgaatgatgaggcaattaatgaccaaattgatagggaaattgaagcaaaaatggaagatgaggggttctaccaagtaaaagctcgagaaaccctagcatcacaatgtagaaacaaaaaacgcaaaacttttgttgaacaggctcttttgcatattatagatcttaagcctcataaagttaggacagttaaggttgaggttcaggatgagagtgctaaccaacttaaaaaggtctttaaaaaaatggtcaaagacgaggaggttgaacaggggcaaacgaaggccttcttttcttcaggtgatgatatagaatattttgaaaatcgtgacattgattggaaaaaattggaaatgccaaaattacaggatgaggttaaatttgagactatcacgcgcaatggcataactcaaatttctactggtgaggataataagaaaaacatcctcaaagaaaaggtcacaaatataccacaaccattgacaccagtcccaccatttacggagaataatccggacactaagcctaaaaaggagcaagttcttgaacaacggaagagggtctgtaggtcatgtggcagcgacaaaccccacaatttcgttgtgtgcaagaaaaaaaatgagctcttgttctgtgtctggtgtggtatagttcactctgagaatcaaggccactcacggaaaatcgaatgccctaagtgcaacaaagcattttctgggatagaaggtcttgaacgccatgcgagtgaatgccctccaaaaaactagtggagggcccagatgattctgggcccgattatgaacctgttcccgattatttgaagattttttcttgggaagatgacttgttacctccaattggaaaagaagctctacctgataatgttatacttcttggtcatattccagttgacaaattagtttctagaacaaaaaaggtcaccgatccactacttggtgttattacgtcctggaaacaggatgagtatgacagtactacatggacagtgaaggcgtacacgaagatgtttgaaaaatttttttataaagaaccatctgactttgtgaacaatgagccagaattgaccatcctttgtgatagggtggtgcttgatgagcatgattatatggctaatagttctatgacgcctataacagcaactgtcaaaaatgtagactcaacaccagcttatccaaaatttcaggagtttgatactgaggaagaatatctcacccgttgtggatggagtgaataccttgatgttattaaggacaaagaaactctcaaccataggccattatggtggtgcttccttaaaaacgaagttctcaagaaaaagaaaattcaagagaatgacattaggatgattttatgtacagatccagtttttactcgaattggagcaatgtttgatcaggaccaaaattcaaaaatgaaaaatatgacagaaacgagggccgctcaagtgggctggacccccttctttggtggacttgaccagcgaatgcggaggttagagaagattgaaaacgcccagtttgtagaaatggattggacccgcttcgatggcactattccaaaggcgctcttttggcgcatccggcagatcagattctttttcctagcgccccggtataaaaccgcagctaacaaggaattgtttgattggtacactaaaaatctgctggagaaaataattctcttaccaactggagaagtgtgtcagataaaaaggggtaatccttccgggcagttctccaccacagttgataacaatatgtgtaatgtgtggctaactacctttgagatagcatggctccaccgcaaacagaaaggtagactccccactcccactgagttgaggaaaaatctcaagtacatttgttatggtgatgacaggctcttggcagtttcaaaggattttgtgtcctacgagcctgacactgttgttaagatgtatgcagatatctttgggatgtgggtgaaaccagaaaatgtgaaaatcagagatagcctagttggtctctcgttctgtgggatgacaattatcaaaaattcgagcaaccgttatgtaggtgttcccaatgtaaataaaattctttctactctcagtactcctacaaaaaggcttccaaatatcgaggcattgtgggggaaattgatctcactgcggatcctttgcgagaatgctgaccccgacgtcagggactacttagataagcaaatccactgcgtcgaggagtatgctgctgctgaagaaatacagttaccagaagtcgggcccgacttctttcagaaaatctggtagagggatggaccgaaatagagcagcatggccgataaggctgggccgcagaagaagagggtatctaggcgtggacgtggccgctctcgttcaaggtcacgctcacgctctcgatcaagaaatcgtgtcaagaaaacagtcacgatagttgaaacaaaaaagaccccaagtaagtcaattctgaaaaaggagttggacaatcacgagaggaaagacagaaaaaggttcaaaaaattggaaaggaaactaaatggaccaaaaattcatgatcgcatggcagttacaactactcttggtgttcttacaggtaattctgataataatttagagaggaagatgagagccttgcttaatccacttctgctaaaatcccagaacaccggagcttcagcgtcaccactctctcttagagcatcacaatattctatgtggaaaatacagcggtgtgttgttaaatttgtgccgttggtgggtgcagcaaatgttgctggaagtgtgtcatttgtatcattagaccaggatgcaacatcatcccaacctgaatcacctgacactataaaagcgaaggtacatgcagaagtcgcaattgggcagaggttcaattggaatgtgcaatccaggtacctggttggaccccgttctggttggtggggtatggatactggtgagtcaccaactgatacggttggaccagctctcgacttctggaacctctacagaacagtaaacacactccagactggctcaacatcacagacgtacactgcgccattgttttctgtggaggtcttcgcagtgtatgtgttctcaggatatgagcctaaacctgctcttgcaaccatgacaaattcaacttttgaaagccagcagggagtaactataacgaatggttctaatggggaacttctgcttaatgtaccaaagcagtcaggtcttgccgagcgactacgtgaaaaagaaacaccgcaacgaggccaaaaccagtcaggcggagttggggaggttctatgggcagttgcatcgggcgctgtggaaggagctgcagaagcactgggcccatggggttggctacttcggggtggctggtgggttattaaaaaactgtttgggcggagcgctgagaatgctaatgatgtctatgtaatgtattcatctattgaggatgctaataaagatagcagaatttaccagacagtcacaagcgcagtaccagtccagcaaggcccactagtccttacccaaatttcatcacccaatgtcaaccaggccggtggagtagtacaagtgggatcgtcacttacaactgactatctgccactttcccaggcgcaagtcccatttttggaaaatgtcctttatactaacactggtcagcctttgacgtccaacaagagtcatacaatgaggataactggatttccggcctcaaaactagtaacatcgtcatcatctcaatggctgggtactgctgacaaaagtgttcaagcgacaaaatggcttatgtctgactacaccgatacaggagtgatctttggtttcccctactctaatgatcaaccaggagaaacattgggcaatatcggtgtgatacacacatcaaagtcgcttttgaaaacagtgacatcacggcaccagcgtaggctgagcatggcaccactaatttcaaccccaataccatcggcttcgagggggccaacacagatgcgtggatgtttcgacaccccctactattggataagggtttgtgataacacatgttctaataaacccacaaacggagccgttacacagcggtataatgcatggggtgtcatggtggtgagcgtagttcacaataaaatctatgttctagccggttatcctgatggccaaaatagagtaccacaacagcaaatggtatgggacacgtttgactgggatgccacatttaccactggcaggatctattcctcaacatggccaggcactgaggaagaagttgaagatgagactgatgctgagtctgatatttctagcctgtttgatccagtgaatgaagtggagcaggattttcattttcagtgtagtcttaagacatcagactacctaaaagaggaggcggatttttggaaggcaaaggcacaacagttactaatggaaaaagcaatggaaaaatcagcttccaatcctcctcttgtccgctttgagaagggcggacctgagcagcaaaaacaacctgctagcagccgcggccacgccgagtaggaacgagggtacagctgcaccttctcaatggagtttttatgccgtaatcaggcttttctccaaaattaaggcaccggggccacgccgagtaggaacgagggtacagtgccgggttgaccctaccttaaaagggcgtccgccggcatgcaatcaccatgtcggggtctggtttagatcacaggcaatcatcctgtgtgtcaatcaggctcttcgggcggttttagagacagtagtttttaaaaccaatttaattttgtattagattaattggcattta

SEQ ID NO: 4 - protein, 1139 aa, chicken astrovirus
MAQAMAGAFSSLNRREDRKSAQVTAGLDKVFSFQGVHELFVRMRALSGTTTAWKALMNSEAVYIKDVKTAFGANGSQIGFFFAETPTTPTWSPDAGIAILTEGEKTCLAAQQARDLRLKASLSTNSSLVHQIMEKTREAKEKTKQLEELQHRIDNMVDTNKVLYQRMEQRHQEKLEALGEKISKLRYDNHQWFMTCEKKDEEIANLKRQLETKKSGYKKMAWDAVAWFVLAILLFSFFSVSEGATPNGTLPIKDVSWNFDDVEKTCMKPDFGCLVMDTWLPHPVLTFEDLMSKCYNTHGNVIPRSAFNSEQLLYDCTKTAHYFNDGHDYIENYHWCEKRLATLVAANCQGDNGVDKIYTQVVEAVAASRKFFQKMALYKLDVWILAIFSIVLAGNKEKIVKLTPFIALGWWFNLPIFLLSTAVNFFPTMALPFIAFQILQPGFLVVTAFTLWLTLTLAAFFWNDGIAILVETSFALFYTILFFVWSMAMTVCVSLQLSLAYQILLFCVSLSVYCGTKFACSQVVITNPDGTTEKVSRVGKVRKAAFQQCKGVLTFLQTRGIIPSTPVKTNSVVVITGKNGAGTGFRFMNYIVTAGHVVTGSEWATAKFGDVSVKIKKEKEIDMCECPDTLVLFKLPKELQSVKPLRLAQNIKSNYMTLHGFCPNFVNPVSFTGWCTIDGPWLNNAFNTTFGNSGAPYCDPDGKLVGIHLGTQGVTSQGFVICDTLRKQFETHYQCSCRREECDQQPAPAPPVQFDYEEFLSKVITGTKVSHQAILKQQEEMLEQIQLVTKILQENGLLAEQKKKGKTKRTARGAKATMTKKYLSKGHFMKMKMLSEEEYQKLVDEGFTADEIKEVVNNLREQAWLEYCIDNDIDDEGAEDWYDQMLNDEAINDQIDREIEAKMEDEGFYQSKARETLASQCKDKKRKTFAEQALLHIIDLKPHKVRTVKVEVQDESANQLKKVFKRMVKDEEVEQGQTKAFFSSGDDIEYFENRDIDWKKLEMPKLQDEVKFETITRNGITQISTGEDNKKNILKEKVTNIPQPLTPVPPFTENNPDTKPKKEQVLEQRKRVCRSCGSDKPHNFVVCKKKNELLFCVWCGIVHSENQGHSRKIECPKCNKAFSGIEGLERHASECPSKN

SEQ ID NO: 5 - protein, 1139 aa, chicken astrovirus
MAQAMAGAFSSLNRREDRKSAQVTTGLDKVFSFQGVHELFVRMRVLYGTSTAWKALMNCEAVYIKDVKTAFGANGSQIGFFFAETPTTPTWSPDAGIAILTEGEKTCLAAQQARDLRLKASLSTNSSLVHQIMERTREAKEKTKQLEELQQRIDNMVDTNKVLYQRMEQRHQEKLEALGEKISKLRHDNHQWFMTCEKKDEEITKLKHQLETKKNGYKKMAWDAVAWLVLAILLFSFFSVSEGATPNGTLPIKEVSWNFDDVEKTCMKPDFGCLVMDTWLPHPVLTFEDLMSKCYNTHGNVIPRSAFSSEQLLYDCTKTAHYFNDGHDYIENYHWCEKRLATLVAANCQGDNGVDKIYTQVVEAVAASRKFFQKMALYKLDVWILAIFSIVLAGNKEKIVKLTPFIALGWWFNLPIFLLSTAVNFFPTMALPFIAFQILQPGFLVVTAFTLWLTLTLAAFFWNDGIAILVETSFALFYTILFFVWSMAMTVCASLQLSLAYQILLFCVSLSVYCGTKFACSQVVITNPDGTTEKVSRVGKVRKAAFQQCKGVLTFLQTRGIIPSTPVKTNSVVVITGKNGAGTGFRFMNYIVTAGHVVTGSEWATAKFGDVSVKIKKEKEIDMCECPDTIVLFKLPKELQGVKPLRLAQNVKSNYMTLHGFCPNFVNPVSFTGWCTIDGPWLNNAFNTTFGNSGAPYCDPDGKLIGIHLGTQGVTSQGFVISDVLRKQFETHYQCSCRKEESVQQPTPAPPAQFDYEEFLSKVITGTKISHQAILKQQEEMLEQIQLVTKILKENGLLAEQKKKGKTKRTARGAKATMTKKYLSKGHFMKMKMLSEEEYQKLVDEGFTADEIKEVVNNLREQAWLEYCIDNDIDDEGAEDWYDQMLNDEAINDQIDREIEAKMEDEGFYQVKARETLASQCRNKKRKTFVEQALLHIIDLKPHKVRTVKVEVQDESANQLKKVFKKMVKDEEVEQGQTKAFFSSGDDIEYFENRDIDWKKLEMPKLQDEVKFETITRNGITQISTGEDNKKNILKEKVTNIPQPLTPVPPFTENNPDTKPKKEQVLEQRKRVCRSCGSDKPHNFVVCKKKNELLFCVWCGIVHSENQGHSRKIECPKCNKAFSGIEGLERHASECPPKN

SEQ ID NO: 6 - protein, 1139 aa, chicken astrovirus
MAQAMVGAFSSLNRREDRKSAQVTTGLDKVFSFQGVHELFVRMRALYGTTTAWKALMNCEAVYIKDVKTAFGANGSQIGFFFAETPTTPTWSPDAGIAILTEGEKTCLAAQQARDLRLKASLSTNSSLVHQIMERTREAKEKTKQLEELQQRIDNMVDTNKVLYQRMEQRHQEKLEALGEKISKLRHDNHQWFMTCEKKDEEITKLKHQLETKKNGYKKMAWDAVAWLVLAILLFSFFSVSEGATPNGTLPIKEVSWNFDDVEKTCMKPDFGCLVMDTWLPHPVLTFEDLMSKCYNTHGNVIPRSAFSSEQLLYDCTKTAHYFNDGHDYIENYHWCEKRLATLVAANCQGDNGVDKIYTQVVEAVAASRKFFQKMALYKLDVWILAIFSIVLAGNKEKIVKLTPFIALGWWFNLPIFLLSTAVNFFPTMALPFIAFQILQPGFLVVTAFTLWLTLTLAAFFWNDGIAILVETSFALFYTILFFVWSMAMTVCASLQLSLAYQILLFCVSLSVYCGTKFACSQVVITNPDGTTEKVSRVGKVRKAAFQQCKGVLTFLQTRGIIPSTPVKTNSVVVITGKNGAGTGFRFMNYIVTAGHVVTGSEWATAKFGDVSVKIKKEKEIDMCECPDTIVLFKLPKELQGVKPLRLAQNVKSNYMTLHGFCPNFVNPVSFTGWCTIDGPWLNNAFNTTFGNSGAPYCDPDGKLIGIHLGTQGVTSQGFVISDVLRKQFETHYQCSCRKEESVQQPTPAPPAQFDYEEFLSKVITGTKISHQAILKQQEEMLEQIQLVTKILKENGLLAEQKKKGKTKRTARGAKATMTKKYLSKGHFMKMKMLSEEEYQKLVDEGFTADEIKEVVNNLREQAWLEYCIDNDIDDEGAEDWYDQMLNDEAINDQIDREIEAKMEDEGFYQVKARETLASQCRNKKRKTFVEQALLHIIDLKPHKVRTVKVEVQDESANQLKKVFKKMVKDEEVEQGQTKAFFSSGDDIEYFENRDIDWKKLEMPKLQDEVKFETITRNGITQISTGEDNKKNILKEKVTNIPQPLTPVPPFTENNPDTKPKKEQVLEQRKRVCRSCGSDKPHNFVVCKKKNELLFCVWCGIVHSENQGHSRKIECPKCNKAFSGIEGLERHASECPPKN

SEQ ID NO: 7 - protein, 519 aa, chicken astrovirus
MPFKKLVEGPDDSGPDYEPVPDYLKIFSWEDDLLPPIGKEALPENVILLGHIPVDKLVSRTKKVTDPLLGVITSWKQDEYDSTTWTVKAYTKMFEKFFYKEPSDFVNNEPELTILCDRVVLDEHDYMANSSMTPITATIKNVDSTPAYPKFQEFDTEEEYLTRCGWSEYLDVIKNKETLNHRPLWWCFLKNEVLKKKKIQENDIRMILCTDPVFTRIGAMFDQDQNSKMKNMTETRAAQVGWTPFFGGLDQRMRRLEKIENAQFVEMDWTRFDGTIPKALFWRIRQIRFFFLAPRYKTAANKELFDWYTKNLLEKIILLPTGEVCQIKRGNPSGQFSTTVDNNMCNVWLTTFEIAWLHRKQKGRLPTPTELRKNLKYICYGDDRLLAVSKDFVVYEPDTVVKMYADVFGMWVKPENVKVRDSLVGLSFCGMTIIKNSNNRYVGVPNVNKILSTLSTPTKRLPNIEALWGKLISLRILCENADPDVRDYLDKQIHCVEEYAAAEDIQLPEVGPDFFQKIW

SEQ ID NO: 8 - protein, 519 aa, chicken astrovirus
MPSKKLVEGPDDSGPDYEPVPDYLKIFSWEDDLLPPIGKEALPDNVILLGHIPVDKLVSRTKKVTDPLLGVITSWKQDEYDSTTWTVKAYTKMFEKFFYKEPSDFVNNEPELTILCDRVVLDEHDYMANSSMTPITATVKNVDSTPAYPKFQEFDTEEEYLTRCGWSEYLDVIKDKETLNHRPLWWCFLKNEVLKKKKIQENDIRMILCTDPVFTRIGAMFDQDQNSKMKNMTETRAAQVGWTPFFGGLDQRMRRLEKIENAQFVEMDWTRFDGTIPKALFWRIRQIRFFFLAPRYKTAANKELFDWYTKNLLEKIILLPTGEVCQIKRGNPSGQFSTTVDNNMCNVWLTTFEIAWLHRKQKGRLPTPTELRKNLKYICYGDDRLLAVSKDFVSYEPDTVVKMYADIFGMWVKPENVKIRDSLVGLSFCGMTIIKNSSNRYVGVPNVNKILSTLSTPTKRLPNIEALWGKLISLRILCENADPDVRDYLDKQIHCVEEYAAAEEIQLPEVGPDFFQKIW

SEQ ID NO: 9 - protein, 519 aa, chicken astrovirus; identical to SEQ ID NO: 8

SEQ ID NO: 10 - protein, 743 aa, chicken astrovirus
MADKAGPQKKRASRRGRGRSRSRSRSRSRSRNRVKKTVTIVETKKTPSKSILKKELDNHERKDRKRFRKLEKKLNGPKIHDRMAVTTTLGVLTGNSDNNLERKMRALLNPLLLKSQNTGASASPLSLRASQYSMWKIQRCVVKFVPLVGAANVAGSVSFVSLDQDATSSQPESPDTIKAKVHAEVSIGQRFNWNVQSRYLVGPRSGWWGMDTGESPTDTVGPALDFWNLYRTVNTLQTGTTSQVYTAPLFSIEVFTVYAFSGYEPKPALATMTNSTFESQQGVTITNGSNGELLLNVPQRSAFAERLREKETPQRAQNQTGGVGEVLWAVASGAVEGAAEALGPWGWLLRGGWWVIKKLFGRTGEDANDVYVMYSSIEDANKDSRIYQTVTGSVQIQQGPLVLTQISSPNVNTSGGVVQVNSTTPNDYLPLSQESYAETPLKKYVLYDSTGNPVDSNMSHTMRITGYPESKLVTSSSVWLGTTGKSVQSTKWLMSDYTNTGVIFGFPYTSAPPGATVGNIGVIHTAKSLIKTIRYRRQNHLPTTPFESSLIPSASKGPSQMLGCFDTPYVWCRVCDNTCSTKPTDGAVTQRYNAWGLLVVSLAHDKVYVLSGYPNSQTAVPVQQLVWDTFDWDANFSTGRIYSAVWPGEDGAEQEGSDTDDADSDISSLFDPMNEVEKDFHFQCSLKTSDYLKEEADFWKAKAQQLLMEKAMEKPSANPPLVRFEKGGPEQQKQPASSRGHAE

SEQ ID NO: 11 - protein, 738 aa, chicken astrovirus
MADKAGPQKKRVSRRGRGRSRSRSRSRSRSRNRVKKTVTIVETKKTPSKSILKKELDNHERKDRKRFKKLERKLNGPKIHDRMAVTTTLGVLTGNSDNNLERKMRALLNPLLLKSQNTGASASPLSLRASQYSMWKIQRCVVKFVPLVGAANVAGSVSFVSLDQDATSSQPESPDTIKAKVHAEVAIGQRFNWNVQSRYLVGPRSGWWGMDTGESPTDTVGPALDFWNLYRTVNTLQTGSTSQTYTAPLFSVEVFAVYVFSGYEPKPALATMTNSTFESQQGVTITNGSNGELLLNVPKQSGLAERLREKETPQRGQNQSGGVGEVLWAVASGAVEGAAEALGPWGWLLRGGWWVIKKLFGRSAENANDVFVMYSSIEDANKDSRIYQTVTSAVPVQQGPLVLTQISSPNVNQAGGVVQVGSSLTTDYLPLSQAQVPFLENVLYTNTGQPLTSNKSHTMRITGFPASKLVTSSSSQWLGTADKSVQATKWLMSDYTDTGVIFGFPYSNDQPGETLGNIGVIHTSKSLLKTVTSRHQRRLSMAPLISTPIPSASRGPTQMRGCFDTPYYWIRVCDNTCSNKPTNGAVTQRYNAWGVMVVSVVHNKIYVLAGYPDGQNRVPQQQMVWDTFDWDATFTTGRIYSSTWPGTEEEVEDETDAESDISSLFDPVNEVEQDFHFQCSLKTSDYLKEEADFWKAKAQQLLMEKAMEKSASNPPLVRFEKGGPEQQKQPASSRGHAE

SEQ ID NO: 12 - protein, 738 aa, chicken astrovirus
MADKAGPQKKRVSRRGRGRSRSRSRSRSRSRNRVKKTVTIVETKKTPSKSILKKELDNHERKDRKRFKKLERKLNGPKIHDRMAVTTTLGVLTGNSDNNLERKMRALLNPLLLKSQNTGASASPLSLRASQYSMWKIQRCVVKFVPLVGAANVAGSVSFVSLDQDATSSQPESPDTIKAKVHAEVAIGQRFNWNVQSRYLVGPRSGWWGMDTGESPTDTVGPALDFWNLYRTVNTLQTGSTSQTYTAPLFSVEVFAVYVFSGYEPKPALATMTNSTFESQQGVTITNGSNGELLLNVPKQSGLAERLREKETPQRGQNQSGGVGEVLWAVASGAVEGAAEALGPWGWLLRGGWWVIKKLFGRSAENANDVYVMYSSIEDANKDSRIYQTVTSAVPVQQGPLVLTQISSPNVNQAGGVVQVGSSLTTDYLPLSQAQVPFLENVLYTNTGQPLTSNKSHTMRITGFPASKLVTSSSSQWLGTADKSVQATKWLMSDYTDTGVIFGFPYSNDQPGETLGNIGVIHTSKSLLKTVTSRHQRRLSMAPLISTPIPSASRGPTQMRGCFDTPYYWIRVCDNTCSNKPTNGAVTQRYNAWGVMVVSVVHNKIYVLAGYPDGQNRVPQQQMVWDTFDWDATFTTGRIYSSTWPGTEEEVEDETDAESDISSLFDPVNEVEQDFHFQCSLKTSDYLKEEADFWKAKAQQLLMEKAMEKSASNPPLVRFEKGGPEQQKQPASSRGHAE

SEQ ID NO: 13 - DNA, 27 bp, artificial
  synthetic oligonucleotide primer
gataaggctgggccgcagaagaagagg

SEQ ID NO: 14 - DNA, 23 bp, artificial
  synthetic oligonucleotide primer
acaaatttaacaacacaccgctg

SEQ ID NO: 15 - DNA, 28 bp, artificial
  synthetic oligonucleotide primer
gggaattcttacaacccaaaacctgggc

SEQ ID NO: 16 - DNA, 31 bp, artificial
  synthetic oligonucleotide primer
gggagctcgggagtatagggtcttcagatgg

SEQ ID NO: 17 - DNA, 31 bp, artificial
  synthetic oligonucleotide primer
gggaattcggaccattgtggcagatcgaagc

SEQ ID NO: 18 - DNA, 29 bp, artificial
  synthetic oligonucleotide primer
gggagctcggtgctgaaccagtacctggc

SEQ ID NO: 19 - DNA, 40 bp, artificial
  synthetic oligonucleotide primer
ggtctagattcttgtctaaagttataacaggaacaaagat

SEQ ID NO: 20 - DNA, 33 bp, artificial
  synthetic oligonucleotide primer
gggagctcgggcttttgattggtagaatccttc

SEQ ID NO: 21 - DNA, 36 bp, artificial
  synthetic oligonucleotide primer
ggccgcgggaattcggcaacactaacggacaacacg

SEQ ID NO: 22 - DNA, 36 bp, artificial
  synthetic oligonucleotide primer
ggaagcttgagctcggaaaaacaaatgtagtttccc

SEQ ID NO: 23 - protein, 37 aa, chicken astrovirus
MAASTSKHYANRSFDNVLSIVCTRCDAVVQSKDARSN

SEQ ID NO: 24 - protein, 35 aa, chicken astrovirus
TVYSCRQHTKHNLESAIGVISVKSVTAIKVRNVKP

SEQ ID NO: 25 - protein, 11 aa, chicken astrovirus
MFEINVVVNDW

SEQ ID NO: 26 - protein, 9 aa, chicken astrovirus
SDVDSIISE

SEQ ID NO: 27 - protein, 114 aa, chicken astrovirus
MARKSTVITAQRAFATTGDYGSQITSNTTPNESYETLKKYDSNVDMYEVTGSNTSAPAVAIIRYRNHPTTFESLKSLVCTDLLLADVSNSTAVLNSAVEDGAQGSTDDMKPSAE

SEQ ID NO: 28 - protein, 107 aa, chicken astrovirus
VKRASTVAVKQGLGSSANFSAPVQAGSLTTQVFLENTNQLTKFASQADADSNDQELSLVTSHRRSMALITPRTRYINNVMVVNIADGNRQMTTSTTEVEETEVQSAS

SEQ ID NO: 29 - protein, 5 aa, chicken astrovirus
MAVSN

SEQ ID NO: 30 - RNA, 24 nt, chicken astrovirus
ccgaaagcgagggugugggcgaug

SEQ ID NO: 31 - RNA, 13 nt, turkey astrovirus 1
ccgaagugggaug

SEQ ID NO: 32 - RNA, 23 nt, turkey astrovirus 2
ccgaaaguguugucggggcgaug

SEQ ID NO: 33 - RNA, 25 nt, duck astrovirus
ccgaagugggcgaguuggggccaug

SEQ ID NO: 34 - RNA, 16 nt, avian nephritis virus 1
ccgaauagaugggaug

SEQ ID NO: 35 - RNA, 21 nt, chicken astrovirus
  polyA_site (21)..(21)
aauuaaauuaauuggcauuua

SEQ ID NO: 36 - RNA, 21 nt, turkey astrovirus 2
  polyA_site (21)..(21)
uaaaaauuuuauuagcauuua

SEQ ID NO: 37 - RNA, 21 nt, duck astrovirus
  polyA_site (21)..(21)
aguagauucaauuggcauuua

SEQ ID NO: 38 - RNA, 21 nt, avian nephritis virus 1
  polyA_site (21)..(21)
uuuugaauuggcuaacuuuua

SEQ ID NO: 39 - RNA, 30 nt, chicken astrovirus
gagugaaugcccuucaaaaaacuaguggag

SEQ ID NO: 40 - protein, 8 aa, chicken astrovirus
MPSKKLVE

SEQ ID NO: 41 - protein, 7 aa, chicken astrovirus
SECPPKN

SEQ ID NO: 42 - RNA, 30 nt, duck astrovirus
uuguucaaaaaacuagaugaggggccugaa

SEQ ID NO: 43 - protein, 8 aa, duck astrovirus
KKLDEGPE

SEQ ID NO: 44 - protein, 4 aa, duck astrovirus
CSKN

SEQ ID NO: 45 - RNA, 29 nt, turkey astrovirus 1
guguucaaaaaacuagauagucacccuga

SEQ ID NO: 46 - protein, 7 aa, turkey astrovirus 1
KKLDSHP

SEQ ID NO: 47 - protein, 4 aa, turkey astrovirus 1
CSKN

SEQ ID NO: 48 - RNA, 30 nt, turkey astrovirus 2
agagugucaaaaaaacuaguagaggggccc

SEQ ID NO: 49 - protein, 9 aa, turkey astrovirus 2
VSKKLVEGP

SEQ ID NO: 50 - protein, 5 aa, turkey astrovirus 2
ECQKN

SEQ ID NO: 51 - RNA, 76 nt, chicken astrovirus
ccuucaaaaaacuaguggaggggcccagaugauucugggcccgauuaugaaccuguccccgacuauuugaagauuu

SEQ ID NO: 52 - RNA, 75 nt, Human astrovirus
gccccaaaaaacuacaaagggccccagaagaccaaggggcccaaaauuaccacucauuagaugcauccaaaucau